SEQUENCE LISTING (109 sequences)

SEQ ID NO: 1 - DNA, 500 bp, artificial
  Sequence of the first arm of the HRPL/CFA6 dsRNA used to concomitantly target HrpL and Cfa6 genes of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccgctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 2 - DNA, 1410 bp, artificial
  Sequence of the CHSA intron used to generate all the inverted repeat from the present invention
ggcgcgcccaatcgatgatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacattttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtaaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtatttggatcctaggtgagtctagaggcgcgcc

SEQ ID NO: 3 - DNA, 500 bp, artificial
  Sequence of the second arm of the HRPL/CFA6 dsRNA used to concomitantly target HrpL and Cfa6 genes of Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcagcggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 4 - DNA, 250 bp, artificial
  Sequence of the first arm of the CFA6-A dsRNA used to target the Cfa6 gene of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccg

SEQ ID NO: 5 - DNA, 250 bp, artificial
  Sequence of the second arm of the CFA6-A dsRNA used to target the Cfa6 gene of Pto DC3000
cggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 6 - DNA, 472 bp, artificial
  Sequence of the first arm of the CFA6-B dsRNA used to target the Cfa6 gene of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccgaatggcaagtattctgccgacagcaactgacacgcgaccatgccgaataccgcgcggccatcttagccaccgatcaagccagcctactcaaagggctggatatgctggcccgcggccgagcctcgcgccatctggtgctgggccgcgccgaccagttgcgacagccggtactggtgtttccaggccaaggaccgctatggccgcggatgactaccggactga

SEQ ID NO: 7 - DNA, 472 bp, artificial
  Sequence of the second arm of the CFA6-B dsRNA used to target the Cfa6 gene of Pto DC3000
tcagtccggtagtcatccgcggccatagcggtccttggcctggaaacaccagtaccggctgtcgcaactggtcggcgcggcccagcaccagatggcgcgaggctcggccgcgggccagcatatccagccctttgagtaggctggcttgatcggtggctaagatggccgcgcggtattcggcatggtcgcgtgtcagttgctgtcggcagaatacttgccattcggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 8 - DNA, 250 bp, artificial
  Sequence of the first arm of the HRPL-A dsRNA used to target the HrpL gene of Pto DC3000
ctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 9 - DNA, 250 bp, artificial
  Sequence of the second arm of the HRPL-A dsRNA used to target the HrpL gene of Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcag

SEQ ID NO: 10 - DNA, 348 bp, artificial
  Sequence of the first arm of the HRPL-B dsRNA used to target the HrpL gene of Pto DC3000
ctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctgagcatctgaatctgatcggccgtcatttggcgaataccggcggacgaagatggctgacggggctgggttgagtcgaggatcacaatcttctgaaacat

SEQ ID NO: 11 - DNA, 348 bp, artificial
  Sequence of the second arm of the HRPL-B dsRNA used to target the HrpL gene of Pto DC3000
atgtttcagaagattgtgatcctcgactcaacccagccccgtcagccatcttcgtccgccggtattcgccaaatgacggccgatcagattcagatgctcagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcag

SEQ ID NO: 12 - DNA, 360 bp, artificial
  Sequence of the first arm of the HRCC dsRNA used to target the HrcC gene of Pto DC3000
gaaggcgtggttctggttcgtggtccggccaaatacgtggagtttgtgcgcgactacagcaagaaagtcgaaaagcccgacgagaaggccgacaagcaagatgttgtcgtgctgccactcaaatacgccaacgcggctgatcggactattcgctaccgtgaccagcagttagtggtggccggtgtcgccagtattcttcaagagctgctggaaagccgttcgcgtggcgaaagcattgacagcgtgaacctgttgccggggcagggcagcagtgttgccaacagcacaggtgtcgcggccgccggcctgccttacaacctgggctccaatggtatcgatacgggagcactgcaacagggc

SEQ ID NO: 13 - DNA, 360 bp, artificial
  Sequence of the second arm of the HRCC dsRNA used to target the HrcC gene of Pto DC3000
gccctgttgcagtgctcccgtatcgataccattggagcccaggttgtaaggcaggccggcggccgcgacacctgtgctgttggcaacactgctgccctgccccggcaacaggttcacgctgtcaatgctttcgccacgcgaacggctttccagcagctcttgaagaatactggcgacaccggccaccactaactgctggtcacggtagcgaatagtccgatcagccgcgttggcgtatttgagtggcagcacgacaacatcttgcttgtcggccttctcgtcgggcttttcgactttcttgctgtagtcgcgcacaaactccacgtatttggccggaccacgaaccagaaccacgccttc

SEQ ID NO: 14 - DNA, 400 bp, artificial
  Sequence of the first arm of the AvrPto/AvrPtoB dsRNA used to target the AvrPto and AvrPtoB genes of Pto DC3000
atgggaaatatatgtgtcggcggatccaggatggcccatcaggtgaactccccagaccgagttagtaacaactcgggtgacgaagataacgtaacgtccagtcaactgctgagcgtcagacatcaacttgcggagtctgctggtgtaccaagagatcagcatgaatttgttagtaaccaagcacctcaaagcctgagaaaatggcgggtatcaatagagcgggaccatcgggcgcttattttgttggccacacagaccccgagccagtatcggggcaagcacacggatccggcagcggcgccagctcctcgaacagtccgcaggttcagccgcgaccctcgaatactcccccgtcgaacgcgcccgcaccgccgccaaccggacgtgagaggctttcacg

SEQ ID NO: 15 - DNA, 400 bp, artificial
  Sequence of the second arm of the AvrPto/AvrPtoB dsRNA used to target the AvrPto and AvrPtoB genes of Pto DC3000
cgtgaaagcctctcacgtccggttggcggcggtgcgggcgcgttcgacgggggagtattcgagggtcgcggctgaacctgcggactgttcgaggagctggcgccgctgccggatccgtgtgcttgccccgatactggctcggggtctgtgtggccaacaaaataagcgcccgatggtcccgctctattgatacccgccattttctcaggctttgaggtgcttggttactaacaaattcatgctgatctcttggtacaccagcagactccgcaagttgatgtctgacgctcagcagttgactggacgttacgttatcttcgtcacccgagttgttactaactcggtctggggagttcacctgatgggccatcctggatccgccgacacatatatttcccat

SEQ ID NO: 16 - DNA, 752 bp, artificial
  Sequence of the first arm of the CYP51 dsRNA used to target the FgCYP51A, FgCYP51B and FgCYP51C genes of Fusarium graminearum
cagcaagtttgacgagtccctggccgctctctaccacgacctcgatatgggcttcacccccatcaacttcatgcttcactgggcccctctcccctggaaccgtaagcgcgaccacgcccagcgcactgttgccaagatctacatggacactatcaaggagcgccgcgccaagggcaacaacgaatccgagcatgacatgatgaagcaccttatgaactctcggtccattgacaatccccgtctttggtagcgatgtcgtatacgattgtcccaactcgaagctcatggaacaaaagaagtttgtcaagtttggccttacgcaaaaagcactcgagtcacacgtccagttaatcgagcgagaggttcttgactacgtcgaaactgatccatccttttctggcagaactagcaccatcgatgtccccaaggcaatggctgagataacaatctttactgcctcacgttctttgcagggtgaggaagttcggagaaaactcactgccgagtttgctgcattggaagcaccgtacaatatggcatcgacccgtacgcttttttcttcgactgcagagataaatacggcgactgctttacctttattctccttggcaaatcaacgactgtctttcttggtcccaagggcaatgactttatcctcaacggcaaacacgccgatctcaacgccgaggacgtttatgggaaacttaccacgcccgtgtttggtgaggaggttgtttatgactgctccaatg

SEQ ID NO: 17 - DNA, 752 bp, artificial
  Sequence of the second arm of the CYP51 dsRNA used to target the FgCYP51A, FgCYP51B and FgCYP51C genes of Fusarium graminearum
cattggagcagtcataaacaacctcctcaccaaacacgggcgtggtaagtttcccataaacgtcctcggcgttgagatcggcgtgtttgccgttgaggataaagtcattgcccttgggaccaagaaagacagtcgttgatttgccaaggagaataaaggtaaagcagtcgccgtatttatctctgcagtcgaagaaaaaagcgtacgggtcgatgccatattgtacggtgcttccaatgcagcaaactcggcagtgagttttctccgaacttcctcaccctgcaaagaacgtgaggcagtaaagattgttatctcagccattgccttggggacatcgatggtgctagttctgccagaaaaggatggatcagtttcgacgtagtcaagaacctctcgctcgattaactggacgtgtgactcgagtgctttttgcgtaaggccaaacttgacaaacttcttttgttccatgagcttcgagttgggacaatcgtatacgacatcgctaccaaagacggggattgtcaatggaccgagagttcataaggtgcttcatcatgtcatgctcggattcgttgttgcccttggcgcggcgctccttgatagtgtccatgtagatcttggcaacagtgcgctgggcgtggtcgcgcttacggttccaggggagaggggcccagtgaagcatgaagttgatgggggtgaagcccatatcgaggtcgtggtagagagcggccagggactcgtcaaacttgctg

SEQ ID NO: 18 - DNA, 667 bp, artificial
  Sequence of the first arm of the HRPG/HRPB/HRCC dsRNA used to target concomitantly the HrpG, HrpB and HrcC genes of Ralstonia species
cgcatgcctggccgtgcagaacgtggaatgtgtccggttcgacgatgccctgacgctgttgcgtgcacggcgtaccgaggcattcagtctgctgctgatcgatgcccagcagttccgcagcgcgggacagctggtgctgtcctggcgcgagtgcaacgccgacatgtgctggccgacgctggtgttcggccagttcgcagtgaagaagcggaagaaggcttccgcctggcgcagcgcctgatccgccattcggatgacgcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgcctcgaccgcgcaggtcaagcgggcgctgac

SEQ ID NO: 19 - DNA, 667 bp, artificial
  Sequence of the second arm of the HRPG/HRPB/ HRCC dsRNA used to target concomitantly the HrpG, HrpB and HrcC genes of Ralstonia species
gtcagcgcccgcttgacctgcgcggtcgaggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgcgtcatccgaatggcggatcaggcgctgcgccaggcggaagccttcttccgcttcttcactgcgaactggccgaacaccagcgtcggccagcacatgtcggcgttgcactcgcgccaggacagcaccagctgtcccgcgctgcggaactgctgggcatcgatcagcagcagactgaatgcctcggtacgccgtgcacgcaacagcgtcagggcatcgtcgaaccggacacattccacgttctgcacggccaggcatgcg

SEQ ID NO: 20 - DNA, 705 bp, artificial
  Sequence of the first arm of the HRPB/HRCC/TssB/XpsR dsRNA used to target concomitantly the HrpB, HrcC, XpsR and TssB genes of Ralstonia species
gcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgccggccgcgcgtgcggagacccatcggcggccgcagagcgaatgcgactggaagcattactttgcggacctgctctatgcgccgaacggcgccgagttcaagctcagcctgtttccgctgcccgcgcagcagatcggcgacacgccctggtcgcgggcattccgcgggcagccggcgatgcttgacaaccaatccgccgcgctcgcgcagagcgcaccggcagccgagtccgtcaacctgctcgacgagatcgtcgagcagagccgcgtcgccaagtcggacgccgagcacgcgcgcgccaaggacatcatcggcgagctggtcaac

SEQ ID NO: 21 - DNA, 705 bp, artificial
  Sequence of the second arm of the HRPB/HRCC/TssB/XpsR dsRNA used to target concomitantly the HrpB, HrcC, XpsR and TssB genes of Ralstonia species
gttgaccagctcgccgatgatgtccttggcgcgcgcgtgctcggcgtccgacttggcgacgcggctctgctcgacgatctcgtcgagcaggttgacggactcggctgccggtgcgctctgcgcgagcgcggcggattggttgtcaagcatcgccggctgcccgcggaatgcccgcgaccagggcgtgtcgccgatctgctgcgcgggcagcggaaacaggctgagcttgaactcggcgccgttcggcgcatagagcaggtccgcaaagtaatgcttccagtcgcattcgctctgcggccgccgatgggtctccgcacgcgcggccggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgc

SEQ ID NO: 22 - DNA, 633 bp, artificial
  Sequence of the first arm of the HRPG/HRPX/RsmA dsRNA used to target concomitantly the HrpG, HrpX, and RsmA genes of Xanthomonas campestris pv. campestris
gtgatggacgccgctgcgaatgaccgccaaggatcggcattcgtactgacgcaggacgcgcggctggcatcacagatcaacgccagcctggcgactctggtcccagaggtcaccatcttttcagacgaactcgaattgctgcgctgcctgcgccactcgccgtgcgagcttctggttttcgatgcccattgcgtggcatcggacgacagtatgatcctttcgacctacttcgcagcgatctctgcgttgtcttacgcagaccgtcttccgatctatacgagcaggatgcttgttggtgcttggccacagggtctgcagcacctccagcaacgtcgcgaggggcaggcagctccggatggcagcgacgacgaagtcgcattgctgggcgccagcgccgatgccttgttgattctggagtatatgttgatcctcactcgccgagtaggcgaaaccctgatgatcggcgacttggtcaccgtgaccgtgctcggcgtcaagggaaatcaggtgcgcattggcattgacgcgcctaaggatgttgccgtgcatcgcgaagaaatctatcagcgcatccagcgcggtgacgagccggttgcgtccggtgcgcatcacaacgacgattgttcggactga

SEQ ID NO: 23 - DNA, 633 bp, artificial
  Sequence of the second arm of the HRPG/HRPX/ RsmA dsRNA used to target concomitantly the HrpG, HrpX, and RsmA genes of Xanthomonas campestris pv. campestris
tcagtccgaacaatcgtcgttgtgatgcgcaccggacgcaaccggctcgtcaccgcgctggatgcgctgatagatttcttcgcgatgcacggcaacatccttaggcgcgtcaatgccaatgcgcacctgatttcccttgacgccgagcacggtcacggtgaccaagtcgccgatcatcagggtttcgcctactcggcgagtgaggatcaacatatactccagaatcaacaaggcatcggcgctggcgcccagcaatgcgacttcgtcgtcgctgccatccggagctgcctgcccctcgcgacgttgctggaggtgctgcagaccctgtggccaagcaccaacaagcatcctgctcgtatagatcggaagacggtctgcgtaagacaacgcagagatcgctgcgaagtaggtcgaaaggatcatactgtcgtccgatgccacgcaatgggcatcgaaaaccagaagctcgcacggcgagtggcgcaggcagcgcagcaattcgagttcgtctgaaaagatggtgacctctgggaccagagtcgccaggctggcgttgatctgtgatgccagccgcgcgtcctgcgtcagtacgaatgccgatccttggcggtcattcgcagcggcgtccatcac

SEQ ID NO: 24 - DNA, 676 bp, artificial
  Sequence of the first arm of the RpoB/RpoC/FusA dsRNA used to target concomitantly the RpoB, RpoC and FusA genes of Pto DC3000 and Pseudomonas syringae CC440
aacacgtatccgcaaggactttagcaagttgccggacgtaatggatgtgccgtatctcttggccatccagctggattcgtatcgcgaattcctgcaggcgggagcgaccaaagatcagttccgcgacgtcggtctgcatgcagccttcaaatccgttttcccgatcatcagctactccggcaatgctgcgctggagtatgtaggttatcgcttgggcgagatcttgaaagacctactgaatttgctgaaaaaccagggtcaagtcgaagagttcgacgcaatccgtatcggacttgcatcgcctgagatgatccgctcttggtcgttcggtgaagttaaaaagccggaaaccatcaactaccgtacgttcaagcctgagcgtgacggtctgttctgcgccaagatctttggtccggtaaaagattacgagtgcctgtgcggtaagtacaaggcatatggctcgtactactccgattagccgttaccgtaacatcggtatcgtcgctcacgtggatgctggtaaaaccaccaccaccgagcgcgtccttttttacaccggcaaaagccacaaaatgggcgaggtgcatgatggcgccgcgaccacggactggatggttcaggagcaggagcgtggtattaccattacttctgctgctatcactgccttttggcggct

SEQ ID NO: 25 - DNA, 676 bp, artificial
  Sequence of the second arm of the RpoB/RpoC/ FusA dsRNA used to target concomitantly the RpoB, RpoC and FusA genes of Pto DC3000 and Pseudomonas syringae CC440
tcaggccaaaaggcagtgatagcagcagaagtaatggtaataccacgctcctgctcctgaaccatccagtccgtggtcgcggcgccatcatgcacctcgcccattttgtggcttttgccggtgtaaaaaaggacgcgctcggtggtggtggttttaccagcatccacgtgagcgacgataccgatgttacggtaacggctaatcggagtagtacgagccatatgccttgtacttaccgcacaggcactcgtaatcttttaccggaccaaagatcttggcgcagaacagaccgtcacgctcaggcttgaacgtacggtagttgatggtttccggctttttaacttcaccgaacgaccaagagcggatcatctcaggcgatgcaagtccgatacggattgcgtcgaactcttcgacttgaccctggtttttcagcaaattcagtaggtctttcaagatctcgcccaagcgataacctacatactccagcgcagcattgccggagtagctgatgatcgggaaaacggatttgaaggctgcatgcagaccgacgtcgcggaactgatctttggtcgctcccgcctgcaggaattcgcgatacgaatccagctggatggccaagagatacggcacatccattacgtccggcaacttgctaaagtccttgcggatacgctgc

SEQ ID NO: 26 - DNA, 681 bp, artificial
  Sequence of the first arm of the SecE/RpoA/RplQ dsRNA used to target concomitantly the SecE, RpoA and RplQ genes of Pto DC3000 and Pseudomonas syringae CC440
aacatgaatcccaaggctgaagcatcagactctcgctttgatttgctgaaatggctcctggtagtcattttggtagtcgtgggtgttgtcggtaatcagtattactctgctgagccgatcctgtaccgtgttctcgctctccttgtgattgccgcagcagctgcatttgtagcgctgcagactggcaagggcaaagctttcttcgttttggcgaaagaagcgcgtgcagagatgatctgcagatttcggtaaatgagttcctgacaccccgccacattgatgtgcaggttgtcagtccaacccgcgccaaaatcactctcgagcctctcgagcgtggtttcggccataccctgggcaacgcgcttcgccgcattttgttgtcctccatgcccggctgtgcagtagtcgaggccgagattgacggtgtactccatgagtacagcgccatcgaaggtgtagcatatgcgtcatcgtaaaagtggacgtcacctgagccgtaccagctctcaccgtaaagccatgtttcaaaacatggcggtgtcgctgttcgagcacgagctgatcaaaactaccctgccgaaagccaaggaactgcgccgcgttgccgagccgctgatcaccctggccaaggaagacagtgttgctaaccgtcgtctggctttcgaccgtactcgttcggct

SEQ ID NO: 27 - DNA, 681 bp, artificial
  Sequence of the second arm of the SecE/RpoA/ RplQ dsRNA used to target concomitantly the SecE, RpoA and RplQ genes of Pto DC3000 and Pseudomonas syringae CC440
tcaggaacgagtacggtcgaaagccagacgacggttagcaacactgtcttccttggccagggtgatcagcggctcggcaacgcggcgcagttccttggctttcggcagggtagttttgatcagctcgtgctcgaacagcgacaccgccatgttttgaaacatggctttacggtgagagctggtacggctcaggtgacgtccacttttacgatgacgcatatgctacaccttcgatggcgctgtactcatggagtacaccgtcaatctcggcctcgactactgcacagccgggcatggaggacaacaaaatgcggcgaagcgcgttgcccagggtatggccgaaaccacgctcgagaggctcgagagtgattttggcgcgggttggactgacaacctgcacatcaatgtggcggggtgtcaggaactcatttaccgaaatctgcagatcatctctgcacgcgcttctttcgccaaaacgaagaaagctttgcccttgccagtctgcagcgctacaaatgcagctgctgcggcaatcacaaggagagcgagaacacggtacaggatcggctcagcagagtaatactgattaccgacaacacccacgactaccaaaatgactaccaggagccatttcagcaaatcaaagcgagagtctgatgcttcagccttgggattcactgc

SEQ ID NO: 28 - DNA, 718 bp, artificial
  Sequence of the first arm of the NadHb/NadHd/ NadHe dsRNA used to target concomitantly the NadHb, NadHd and NadHe genes of Xanthomonas species
aacagaagaagggagcgctggaatgggagtgattcagaccctggatcgtctgatgaccaacccgatgccggaaggccgggtcgaagacatcctgcgcccggaaggcgaaaacccgttgctcgaaaagggctacgtgaccaccagcgtcgatgcgctgttgaactgggcgcgtacaggttcgatgtggccgatgacctttggtctggcctgctgtgcagtcgagatgatgcagatcgtgagtgagtaccgccaggcaaccgatgccttcgcgagcaatcctgtggaaagcaagcaggaaatccgcaattacacgatgaacttcggcccgcagcatcctgcggcgcacggcgtattgcgcctgatcctggaaatggacggcgaaaccgtggtgcgtgccgacccgcatatcggcctgctgcaccgtggcaccgagaagctggccgagtccaagccgttcaaccagtcggttccgtacagcatcgacgggtaatttcgaagcggcgcgcgacgtcgatccgcaggtagtgctgagcgacaagacgcgcgcgcacatcgatcattggctgagcaagttcccgcccgaccgcaagcgttcggccgtgttgcagggtctgcatgccgcgcaggaacagaaccagggttggttgaccgacgagctgatcgtgggcgtggccaagtatctggagctgccgccggtgtgggcctacgaagtggggct

SEQ ID NO: 29 - DNA, 718 bp, artificial
  Sequence of the second arm of the NadHb/NadHd/ NadHe dsRNA used to target concomitantly the NadHb, NadHd and NadHe genes of Xanthomonas species
tcagccacttcgtaggcccacaccggcggcagctccagatacttggccacgcccacgatcagctcgtcggtcaaccaaccctggttctgttcctgcgcggcatgcagaccctgcaacacggccgaacgcttgcggtcgggcgggaacttgctcagccaatgatcgatgtgcgcgcgcgtcttgtcgctcagcactacctgcggatcgacgtcgcgcgccgcttcgaaattacccgtcgatgctgtacggaaccgactggttgaacggcttggactcggccagcttctcggtgccacggtgcagcaggccgatatgcgggtcggcacgcaccacggtttcgccgtccatttccaggatcaggcgcaatacgccgtgcgccgcaggatgctgcgggccgaagttcatcgtgtaattgcggatttcctgcttgctttccacaggattgctcgcgaaggcatcggttgcctggcggtactcactcacgatctgcatcatctcgactgcacagcaggccagaccaaaggtcatcggccacatcgaacctgtacgcgcccagttcaacagcgcatcgacgctggtggtcacgtagcccttttcgagcaacgggttttcgccttccgggcgcaggatgtcttcgacccggccttccggcatcgggttggtcatcagacgatccagggtctgaatcactcccattccagcgctcccttcttcctgc

SEQ ID NO: 30 - DNA, 677 bp, artificial
  Sequence of the first arm of the DnaA/DnaE1/ DnaE2 dsRNA used to target concomitantly the DnaA, DnaE1 and DnaE2 genes of Xanthomonas species
aacaatgcttggccccgctgtctggaacgtctcgaagctgaattcccgcccgaggatgtccacacctggttgaaacccctgcaggccgaagatcgcggcgacagcatcgtgctgtacgcgccgaacgccttcattgttgagcaggtccgcgagcgatacctgccgcgcatccgcgagttgctggcatatttcgccggcaatggcgaggtggcgctggcggtcggctcccgtcgatcgcgcgaagccttcctcaagccgttggcccaggtggtcaatgtcgtcgaacggcgtcagacattgccggtactggcgaacttgctggtgcaggtgaacaacggccagctgtcgctgacggggaccgacctggaagtcgaaatgatctcgcgcaccatggtcgaggacgcccaggacggcgaaaccacgatcccggcgcgcaagctgttcgacatcctggcatatgtccacttcccgcttcgtccatctgcacgtccacaccgagttctcgttggcggattccaccatccgcgtgcccgagaaaccggatcaggctgacccgaaaaaagccaagcaggccaacctgctgagccgcgcggtcgaactcgacttgcccgcgctggcggtcaccgacctgaacaacctgttcgccctggtcaagttctacaaggccgccgaaggct

SEQ ID NO: 31 - DNA, 677 bp, artificial
  Sequence of the second arm of the DnaA/DnaE1/ DnaE2 dsRNA used to target concomitantly the DnaA, DnaE1 and DnaE2 genes of Xanthomonas species
tcagttcggcggccttgtagaacttgaccagggcgaacaggttgttcaggtcggtgaccgccagcgcgggcaagtcgagttcgaccgcgcggctcagcaggttggcctgcttggcttttttcgggtcagcctgatccggtttctcgggcacgcggatggtggaatccgccaacgagaactcggtgtggacgtgcagatggacgaagcgggaagtggacatatgccaggatgtcgaacagcttgcgcgccgggatcgtggtttcgccgtcctgggcgtcctcgaccatggtgcgcgagatcatttcgacttccaggtcggtccccgtcagcgacagctggccgttgttcacctgcaccagcaagttcgccagtaccggcaatgtctgacgccgttcgacgacattgaccacctgggccaacggcttgaggaaggcttcgcgcgatcgacgggagccgaccgccagcgccacctcgccattgccggcgaaatatgccagcaactcgcggatgcgcggcaggtatcgctcgcggacctgctcaacaatgaaggcgttcggcgcgtacagcacgatgctgtcgccgcgatcttcggcctgcaggggtttcaaccaggtgtggacatcctcgggcgggaattcagcttcgagacgttccagacagcggggccaagcatctgc

SEQ ID NO: 32 - DNA, 717 bp, artificial
  GFP reporter sequence contained in the GFPpPNpt plasmid
atgagtaagggggaggagctctttacaggggtggtacccatactcgtcgagctcgacggggacgtgaacggtcagaagttttcggtaagcggggaaggggagggggacgcgacgtatgggaagctgacactcaagttcatatgtacaacaggaaaactgccggtcccttggcccacgctcgttacaacattaacatacggggtgcagtgtttctcgaggtatccggaccacatgaagcaacacgatttctttaaaagcgcaatgccagaggggtacgttcaagagaggacgattttctataaggacgatggtaattataaaacccgggcggaggttaaattcgagggggacacactggtgaacaggatagaattgaaggggatagacttcaaggaggacggcaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataa

SEQ ID NO: 33 - DNA, 21 bp, artificial
  Primer sequence of Cfa6-Forward used for LMW Northern Blot
caacaacttgccgggatcctg

SEQ ID NO: 34 - DNA, 22 bp, artificial
  Primer sequence of Cfa6-Reverse used for LMW Northern Blot
ggcttgcatccgacagaatcag

SEQ ID NO: 35 - DNA, 21 bp, artificial
  Primer sequence of HrpL-Forward used for LMW Northern Blot
cacttcgtcttcccagctttc

SEQ ID NO: 36 - DNA, 24 bp, artificial
  Primer sequence of HrpL-Forward used for LMW Northern Blot
tttatccaaaagcgggtgatgaac

SEQ ID NO: 37 - DNA, 21 bp, artificial
  Primer sequence of miR159 probe used for LMW Northern Blot
aaacctaacttccctcgagat

SEQ ID NO: 38 - DNA, 19 bp, artificial
  Primer sequence of GyrA-Fwd used for RT-qPCR
aactgctgggtgagtacca

SEQ ID NO: 39 - DNA, 19 bp, artificial
  Primer sequence of GyrA-Rev used for RT-qPCR
gagctcttcgcggatcact

SEQ ID NO: 40 - DNA, 20 bp, artificial
  Primer sequence of CFA6-Fwd used for RT-qPCR
gtcttcatctttcccggtca

SEQ ID NO: 41 - DNA, 20 bp, artificial
  Primer sequence of CFA6-Rev used for RT-qPCR
gtctcgatctggtcgatggt

SEQ ID NO: 42 - DNA, 22 bp, artificial
  Primer sequence of HrpL-Fwd used for RT-qPCR
cgagtcattcaggccattgatt

SEQ ID NO: 43 - DNA, 22 bp, artificial
  Primer sequence of HrpL-Rev used for RT-qPCR
gtttcctgataattgccgtcca

SEQ ID NO: 44 - DNA, 20 bp, artificial
  Primer sequence of ProC-Fwd used for RT-qPCR
cgcagatgatgaaaagcgtc

SEQ ID NO: 45 - DNA, 19 bp, artificial
  Primer sequence of ProC-Rev used for RT-qPCR
agtcaggctggcacaggtg

SEQ ID NO: 46 - DNA, 20 bp, artificial
  qPCR primer RpoB-Fwd for bacterial transcript RpoB
gtaggtctggtccgtgttga

SEQ ID NO: 47 - DNA, 20 bp, artificial
  Primer sequence of RpoB-Rev used for RT-qPCR
gcaagtaatctcggacagcg

SEQ ID NO: 48 - DNA, 20 bp, artificial
  Primer sequence of Cyp3-Forward used for LMW Northern Blot
atccgagcatgacatgatga

SEQ ID NO: 49 - DNA, 20 bp, artificial
  Primer sequence of Cyp3-Reverse used for LMW Northern Blot
gtacgggtcgatgccatatt

SEQ ID NO: 50 - DNA, 21 bp, artificial
  Probe preparation for northern blot analysis U6
aggggccatgctaatcttctc

SEQ ID NO: 51 - DNA, 22 bp, artificial
  Primer sequence of Tomato Ubi-Fwd used for RT-qPCR
ggacggacgtactctagctgat

SEQ ID NO: 52 - DNA, 20 bp, artificial
  Primer sequence of Tomato Ubi-Rev used for RT-qPCR
agctttcgacctcaagggta

SEQ ID NO: 53 - DNA, 20 bp, artificial
  Primer sequence of Pto GFP-Fwd used for RT-qPCR
tggaagcgttcaactagcag

SEQ ID NO: 54 - DNA, 20 bp, artificial
  Primer sequence of Pto GFP-Rev used for RT-qPCR
aaagggcagattgtgtggac

SEQ ID NO: 55 - DNA, 20 bp, artificial
  Primer sequence of IR-CFA6/HRPL-Fwd used for RT-qPCR
gttcatcacccgcttttgga

SEQ ID NO: 56 - DNA, 20 bp, artificial
  Primer sequence of IR-CFA6/HRPL-Rev used for RT-qPCR
cccctctttctaccttccca

SEQ ID NO: 57 - DNA, 21 bp, artificial
  Primer sequence of Ath-Ubi-Fwd used for RT-qPCR
tgaagtcgtgagacagcgttg

SEQ ID NO: 58 - DNA, 20 bp, artificial
  Primer sequence of Ath-Ubi -Rev used for RT-qPCR
gggctttctcattgttggtc

SEQ ID NO: 59 - DNA, 35 bp, artificial
  HRPL-pDON207-F for Cloning of WT HRPL and mut HRPL in pDON207-attB1/B2
aaaaagcaggcttcatgtttcagaagattgtgatc

SEQ ID NO: 60 - DNA, 34 bp, artificial
  HRPL-pDON207-Rev for Cloning of WT HRPL and mut HRPL in pDON207-attB1/B2
agaaagctgggtcggcgaacgggtcgatttgctg

SEQ ID NO: 61 - DNA, 21 bp, artificial
  Primer dcl2-1-WT-fwd for genotyping dcl2-1 allele
tgaatcatctggaagaggtgg

SEQ ID NO: 62 - DNA, 19 bp, artificial
  Primer dcl2-1-mut-fwd for genotyping dcl2-1 allele
attttgccgatttcggaac

SEQ ID NO: 63 - DNA, 21 bp, artificial
  Primer dcl2-1-WT-Rev for genotyping dcl2-1 allele
tgaatcatctggaagaggtgg

SEQ ID NO: 64 - DNA, 21 bp, artificial
  Primer dcl3-1-fwd for genotyping dcl3-1 allele
acaggtaaccttgccatgttg

SEQ ID NO: 65 - DNA, 21 bp, artificial
  Primer dcl3-1-Rev for genotyping dcl3-1 allele
tggaaaagtttgctacaacgg

SEQ ID NO: 66 - DNA, 22 bp, artificial
  primer LBa1
tggttcacgtagtgggccatcg

SEQ ID NO: 67 - DNA, 23 bp, artificial
  Primer dcl4-2-G8605 Fwd for genotyping dcl4-2 allele
ggctgcacagctgatgattacaa

SEQ ID NO: 68 - DNA, 27 bp, artificial
  Primer dcl4-2-G8605 Rev for genotyping dcl4-2 allele
gccgctcgagatcatcagcaaaggaat

SEQ ID NO: 69 - DNA, 28 bp, artificial
  Primer GABI-8474-LP
ataataacgctgcggacatctacatttt

SEQ ID NO: 70 - DNA, 19 bp, artificial
  Northern blot analysis IR-HHR Fwd Primer
ggcatcacagatcaacgcc

SEQ ID NO: 71 - DNA, 18 bp, artificial
  Northern blot analysis IR-HHR Rev Primer
actgtcgtccgatgccac

SEQ ID NO: 72 - DNA, 20 bp, artificial
  RT-qPCR LuxA Fwd
cggagtttggtttgcttggt

SEQ ID NO: 73 - DNA, 20 bp, artificial
  RT-qPCR LuxA Rev
caagttggcgtactggatgg

SEQ ID NO: 74 - DNA, 20 bp, artificial
  RT-qPCR LuxB Fwd
gcggaggaagcttgcttatt

SEQ ID NO: 75 - DNA, 20 bp, artificial
  RT-qPCR LuxB Rev
tgatattcaaccgggcgatt

SEQ ID NO: 76 - DNA, 35 bp, artificial; identical to SEQ ID NO: 59
  HRPL-pDON207-Fwd

SEQ ID NO: 77 - DNA, 34 bp, artificial; identical to SEQ ID NO: 60
  HRPL-pDON207-Rev

SEQ ID NO: 78 - DNA, 37 bp, artificial
  T7 Fwd CFA6/HRPL
taatacgactcactatagggagatgaacaagcgctac

SEQ ID NO: 79 - DNA, 37 bp, artificial
  T7 Rev CFA6/HRPL
taatacgactcactatagggagcagggcgtttatcca

SEQ ID NO: 80 - DNA, 37 bp, artificial
  T7 Fwd CYP51
taatacgactcactatagggagcagcaagtttgacga

SEQ ID NO: 81 - DNA, 37 bp, artificial
  T7 Rev CYP51
taatacgactcactatagggaggcagcaaactcggca

SEQ ID NO: 82 - DNA, 43 bp, artificial
  T7 Fwd Dc3000_FusA
taatacgactcactatagggagatggctcgtactactccgatt

SEQ ID NO: 83 - DNA, 43 bp, artificial
  T7 Rev Dc3000_FusA
taatacgactcactatagggaggccaaaaggcagtgatagcag

SEQ ID NO: 84 - DNA, 42 bp, artificial
  T7 Fwd Dc3000_SecE
taatacgactcactatagggagtgaatcccaaggctgaagca

SEQ ID NO: 85 - DNA, 42 bp, artificial
  T7 Rev Dc3000_SecE
taatacgactcactatagggagatctctgcacgcgcttcttt

SEQ ID NO: 86 - DNA, 43 bp, artificial
  T7 Fwd Dc3000_GyrB
taatacgactcactatagggagatgagcgagaacaacacgtac

SEQ ID NO: 87 - DNA, 43 bp, artificial
  T7 Rev Dc3000_GyrB
taatacgactcactatagggagccgtatggatggtgatgctga

SEQ ID NO: 88 - DNA, 213 bp, artificial
  First strand XC_RS06155  XC_1225
atgcatcgacatgttcatgagcccgaaatcctagcagacccggcagccttcgaagctgccgaacattggcggcaacgcgatcttgcggtggtgtggcacccctgcacccagatgcgcgagcacccgcacacactacccctggtgccgatcgcacgtggcgacggcgcctggctgatcggccacgatggccgccactatctggatgcggtcagc

SEQ ID NO: 89 - DNA, 213 bp, artificial
  Second strand XC_RS06155  XC_1225
gctgaccgcatccagatagtggcggccatcgtggccgatcagccaggcgccgtcgccacgtgcgatcggcaccaggggtagtgtgtgcgggtgctcgcgcatctgggtgcaggggtgccacaccaccgcaagatcgcgttgccgccaatgttcggcagcttcgaaggctgccgggtctgctaggatttcgggctcatgaacatgtcgatgcat

SEQ ID NO: 90 - DNA, 189 bp, artificial
  First strand XC_RS02265 = XC_0447
atgagcgttgacgctgccgcgccgacttccgcctccactccgccgcaaccgcctgcgcctccccagttccccaccttgctcggccacccccggccgctgtggatgctgttcatgaccgaattctgggaacgctttgcgttctacggcattcgttgggcgctggtgctgtacatcgtggcgcagttcttc

SEQ ID NO: 91 - DNA, 189 bp, artificial
  Second strand XC_RS02265 = XC_0447
gaagaactgcgccacgatgtacagcaccagcgcccaacgaatgccgtagaacgcaaagcgttcccagaattcggtcatgaacagcatccacagcggccgggggtggccgagcaaggtggggaactggggaggcgcaggcggttgcggcggagtggaggcggaagtcggcgcggcagcgtcaacgctcat

SEQ ID NO: 92 - DNA, 196 bp, artificial
  First strand XC_RS18260 = XC_3609
atgagcgatgtccttccgatcattctttccggcggctccggcacgcgcctgtggccgttgtcgcgcgaatcgtacccgaagcagttcctgccgctggtcggcgagcacagcatgctgcaggccacctggctgcgctccgctccggtggcggcgcacgcacccatcgtggtggccaacgaagagcaccgcttcatgg

SEQ ID NO: 93 - DNA, 196 bp, artificial
  Second strand XC_RS18260 = XC_3609
ccatgaagcggtgctcttcgttggccaccacgatgggtgcgtgcgccgccaccggagcggagcgcagccaggtggcctgcagcatgctgtgctcgccgaccagcggcaggaactgcttcgggtacgattcgcgcgacaacggccacaggcgcgtgccggagccgccggaaagaatgatcggaaggacatcgctcat

SEQ ID NO: 94 - DNA, 189 bp, artificial
  First strand XC_RS11930 = XC_2375
gagccgtcgcatcattccctgcctggacgtgcgcgatggccgcgtggtcaagggcgtcaagttccgcgaccacatcgacatgggcgacatcgtcgagttggcgatgcgctaccgcgaccagggcgcggacgagttggtgttctacgacattggcgccagcccggaagggcgttcggtggactatgcgtg

SEQ ID NO: 95 - DNA, 189 bp, artificial
  Second strand XC_RS11930 = XC_2375
cacgcatagtccaccgaacgcccttccgggctggcgccaatgtcgtagaacaccaactcgtccgcgccctggtcgcggtagcgcatcgccaactcgacgatgtcgcccatgtcgatgtggtcgcggaacttgacgcccttgaccacgcggccatcgcgcacgtccaggcagggaatgatgcgacggctc

SEQ ID NO: 96 - DNA, 196 bp, artificial
  First strand XC_RS17005 = XC_3357
atggcaaagactcatgaaatcaaggtcgagcgccgcgcagacgaggggaagggtgcgagccgccgcctgcgtcacgctggcgtcattccggccatcgtttacggtggcgaactcgagccggtcagcatccagctcaaccacgagcagatctggcttgcgcagcagaacgagtggttctactcgtcgatcctcgacc

SEQ ID NO: 97 - DNA, 196 bp, artificial
  First strand XC_RS17005 = XC_3357
ggtcgaggatcgacgagtagaaccactcgttctgctgcgcaagccagatctgctcgtggttgagctggatgctgaccggctcgagttcgccaccgtaaacgatggccggaatgacgccagcgtgacgcaggcggcggctcgcacccttcccctcgtctgcgcggcgctcgaccttgatttcatgagtctttgccat

SEQ ID NO: 98 - DNA, 44 bp, artificial
  T7 Fwd XC_RS06155  XC_1225
taatacgactcactatagggagatgcatcgacatgttcatgagc

SEQ ID NO: 99 - DNA, 42 bp, artificial
  T7 Rev XC_RS06155  XC_1225
taatacgactcactatagggaggctgaccgcatccagatagt

SEQ ID NO: 100 - DNA, 40 bp, artificial
  T7 Fwd XC_RS02265 = XC_0447
taatacgactcactatagggagatgagcgttgacgctgcc

SEQ ID NO: 101 - DNA, 43 bp, artificial
  T7 Rev XC_RS02265 = XC_0447
taatacgactcactatagggaggaagaactgcgccacgatgta

SEQ ID NO: 102 - DNA, 41 bp, artificial
  T7 Fwd XC_RS18260 = XC_3609
taatacgactcactatagggagatgagcgatgtccttccga

SEQ ID NO: 103 - DNA, 40 bp, artificial
  T7 Rev XC_RS18260 = XC_3609
taatacgactcactatagggagccatgaagcggtgctctt

SEQ ID NO: 104 - DNA, 40 bp, artificial
  T7 Fwd XC_RS11930 = XC_2375
taatacgactcactatagggaggagccgtcgcatcattcc

SEQ ID NO: 105 - DNA, 41 bp, artificial
  T7 Rev XC_RS11930 = XC_2375
taatacgactcactatagggagcacgcatagtccaccgaac

SEQ ID NO: 106 - DNA, 43 bp, artificial
  T7 Fwd XC_RS17005 = XC_3357
taatacgactcactatagggagatggcaaagactcatgaaatc

SEQ ID NO: 107 - DNA, 42 bp, artificial
  T7 Rev XC_RS17005 = XC_3357
taatacgactcactatagggagggtcgaggatcgacgagtag

SEQ ID NO: 108 - DNA, 500 bp, Artificial
  Sequence of the first arm of the LuxA/LuxB dsRNA used to target concomitantly the LuxA and LuxB genes of Pto DC3000
atgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctg

SEQ ID NO: 109 - DNA, 500 bp, Artificial
  Sequence of the second arm of the LuxA/LuxB dsRNA used to target concomitantly the LuxA and LuxB genes of Pto DC3000
caggatgatgagttgtaatgatgtgatttaatgaaccaattttaattttctctgttaaaccgagcagaaaaccagaaacagtcagaggagcgccgacaacaccattatctgaaaaatgattttcatacactaaaatctgttcaaaattcaacttatcaacatactccgttatttcctgcatgcgaactatactttgttcttgaacagttgttgaattgatgaagttaaggaagaacaatccaaatttcatctggatgggctgtgggaagaacaatagcggcagttcctacattcaattttttagtcgcgccaagtaaatatgcagcagcgacataagggttaccaagcaaaccaaactccgtgaaatgatgctccagtaaccatacggtatcaaaaccacactcctcagagatgcgacctaatttaaccaaacgtttcattacctctgtttgagaaaattggggaggttggtatgtaagcaaaaagtttccaaatttcat

(19) United States
(12) Patent Application Publication
(10) Pub. No.: US 2021/0324394 A1
(43) Pub. Date: Oct. 21, 2021

(54) RNA-BASED BIOCONTROL METHODS TO PROTECT PLANTS AGAINST PATHOGENIC BACTERIA AND / OR PROMOTE BENEFICIAL EFFECTS OF SYMBIOTIC AND COMMENSAL BACTERIA

(71) Applicants: CENTRE NATIONAL DE LA RECHERCHE SCIENTIFIQUE (CNRS), Paris (FR); ECOLE NORMALE SUPERIEURE, Paris (FR); INSTITUT NATIONAL DE LA SANTE ET DE LA RECHERCHE MEDICALE (INSERM), Paris (FR)

(72) Inventors: Lionel NAVARRO, Antony (FR); Meenu SINGLA RASTOGI, Meerut (IN)

(21) Appl. No.: 17/268,951

(22) PCT Filed: Aug. 19, 2019
(86) PCT No.: PCT/EP2019/072169

§ 371 Date: Feb. 16, 2021

(30) Foreign Application Priority Data
Aug. 17, 2018 (EP) 18306124.1

(51) Int. Cl. C12N 15/82; A01N 63/60

(52) U.S. Cl. CPC C12N 15/8218; A01N 63/60; C12N 15/8281

(57) ABSTRACT

The present invention pertains to the field of agriculture. The invention relates to a method to inhibit gene expression in bacteria, which is referred to here as Antibacterial Gene Silencing (AGS). In particular embodiments, the method is used to protect plants against pathogenic bacteria by targeting pathogenicity factors and/or essential genes in a sequence-specific manner via small non-coding RNAs. The method can also be used to enhance beneficial effects and/or growth of plant-associated symbiotic or commensal bacteria. The invention involves either the generation of stable transgenic plants that constitutively express antibacterial small RNAs or, alternatively, the exogenous delivery of these small RNA entities onto plants, either in the form of RNA extracts or embedded into plant extracellular vesicles (EVs), which were found to be effective in reducing bacterial pathogenicity. The invention also describes a method to identify in a rapid, reliable and cost-effective manner, small RNAs that possess antibacterial activity and that can be further exploited for RNA-based biocontrol applications to confer plant protection against pathogenic bacteria. In addition, the latter approach is instrumental to rapidly characterize any genes from any bacterial species.

### SUMMARY OF THE INVENTION

The present invention pertains to the field of agriculture. The invention relates to a method to inhibit gene expression in bacteria, which is referred to here as Antibacterial Gene Silencing (AGS). In particular embodiments, the method is used to protect plants against pathogenic bacteria by targeting pathogenicity factors and/or essential genes in a sequence-specific manner via small non-coding RNAs. The method can also be used to enhance beneficial effects and/or growth of plant-associated symbiotic or commensal bacteria. The invention involves either the generation of stable transgenic plants that constitutively express antibacterial small RNAs or, alternatively, the exogenous delivery of these small RNA entities onto plants, either in the form of RNA extracts or embedded into plant extracellular vesicles (EVs), which were found to be effective in reducing bacterial pathogenicity. The invention also describes a method to identify in a rapid, reliable and cost-effective manner, small RNAs that possess antibacterial activity and that can be further exploited for RNA-based biocontrol applications to confer plant protection against pathogenic bacteria. In addition, the latter approach is instrumental to rapidly characterize any genes from any bacterial species.

### PRIOR ART DESCRIPTION

### Overview of the Plant Immune System

The first layer of the plant immune system involves the recognition of Pathogen- or Microbe-Associated Molecular Patterns (PAMPs or MAMPs), which are conserved microbial signatures that are sensed by surface-localized Pattern-Recognition Receptors (PRRs) (1). Upon ligand binding, these receptors initiate a complex phosphorylation cascade at the PRR complex that leads to PAMP-triggered immunity (PTI) (1). To enable disease, pathogens secrete effectors that suppress PTI (2). For instance, the Gram-negative bacterium Pseudomonas syringae pv. tomato strain DC3000 (Pto DC3000) injects 36 type-III secreted effectors into plant cells to dampen PTI (3). This bacterium also produces coronatine (COR), a phytotoxin that is essential for its pathogenicity (4). Plants have evolved disease resistance (R) proteins that can perceive the presence of pathogen effectors to trigger a host counter-counter defense (5). Most R proteins belong to the nucleotide-binding domain (NBD), leucine-rich repeat (NLR) superfamily, which are also present in animals (2, 5). They recognize, directly or indirectly, pathogen effectors and mount Effector-triggered immunity (ETI), a potent immune response that significantly overlaps with PTI, although with a stronger amplitude (6, 7).

### Post-Transcriptional Gene Silencing (PTGS) Controls Host-Pathogen Interactions

PTGS is a conserved post-transcriptional gene regulatory mechanism that has been extensively characterized as a natural antiviral defense response in plants by targeting and degrading viral transcripts (8). The core mechanism of RNA silencing or RNA interference (RNAi) in plants involves the recognition and processing of double-stranded RNAs (dsRNAs) by RNase III enzyme DICER-LIKE (DCL) proteins leading to the production of 20-25 nt long short interfering RNAs (siRNAs) duplexes. These siRNA duplexes associate with an Argonaute (AGO) protein, the central component of the RNA-induced silencing complex (RISC). Subsequent strand separation on the AGO protein forms a mature RISC composed of AGO and a single-stranded RNA, the guide strand, while the passenger strand is degraded. The guide small RNA directs AGO-RISC onto sequence complementary mRNA targets leading to their endonucleolytic cleavage and/or translational inhibition. During the last decade, several endogenous short interfering RNA (siRNAs) and microRNAs (miRNAs) were additionally found to orchestrate PTI and ETI responses against non-viral pathogens (9), implying a key role of PTGS in the regulation of the plant immune system.

In plants, mobile small RNAs can trigger non-cell autonomous silencing in adjacent cells as well as in distal tissues (10). They are notably important to prime antiviral defense ahead of the infection front (10). Non-cell autonomous silencing is also critical for the translocation of silencing signals between plant cells and their interacting non-viral pathogenic, parasitic or symbiotic organisms—excluding bacteria, which have not been shown to be targeted by this process (11). This natural cross-kingdom regulatory mechanism has been notably recently characterized in plant-fungal interactions (12-17). For instance, specific plant miRNAs were found to be exported into the hyphae of the fungal pathogen Verticillium dahliae to trigger silencing of virulence factors (14, 17). On the other hand, endogenous B. cinerea small RNAs can be exported into plant cells to silence plant defense genes (16), highlighting bi-directional cross-kingdom RNAi between plant and fungal pathogens. Although very little is known about the mechanisms of small RNA/dsRNA trafficking between host cells and fungal cells, the presence of numerous vesicles in the extrahaustorial matrix suggests that they may transfer silencing signals between the two organisms (18). Consistent with this hypothesis, two recent studies provide evidence that plant extracellular vesicles (EVs) are essential to deliver antifungal small RNAs into B. cinerea cells as well as antioomycete small RNAs into Phytophthora capsici cells (17, 19).

### Cross-Kingdom RNAi can be Exploited to Confer Protection Against Pathogens Possessing a Canonical RNA Silencing Machinery

The biological relevance of cross-kingdom RNAi has been initially demonstrated by expressing dsRNAs bearing homologies to vital or pathogenicity factors from a given parasite or pest provided that they possess a canonical RNAi machinery (e.g. functional DCL and AGO proteins). So far, this Host-Induced Gene Silencing (HIGS) technology has been successfully used to protect plants from invasion and predation of insects, nematodes, oomycetes, fungi and parasitic plants (WO 2012/155112, WO 2012/155109, CA 2 799 453, EP 2 405 013, US 2013/177539, 15, 20, 21)). For example, HIGS confers full protection against Fusarium graminearum and B. cinerea and this phenomenon is fully recapitulated by spraying relevant exogenous dsRNAs or siRNAs onto wild type plants prior fungal infections (15, 20, 21). The latter phenomenon is referred to as Spray-Induced Gene Silencing (SIGS) and is reminiscent of ‘environmental RNAi’, a process involving the uptake of RNAs from the environment initially described in Caenorhabditis elegans and in some insects (15, 21, 22). HIGS/SIGS is thus considered as a powerful complement, or even sometimes an alternative, to conventional breeding or genetic engineering designed to introduce R genes or PAMP receptors in agriculturally relevant crops (5, 23, 24). Furthermore, this technology provides a more durable and environmental friendly plant protection solution that will likely contribute to a reduced use of agrochemicals, which can have, in some instances, significant impact on human health and on the environment.

### Current Limitation of HIGS/SIGS Technologies

HIGS/SIGS technologies are limited by the fact that they have only been shown to be functional against plant pathogens and parasites that possess a canonical RNA silencing machinery. For example, SIGS against F. graminearum relies at least in part on the uptake of dsRNAs and further processing by the fungal DICER-LIKE 1 protein (21). So far, there is no example of HIGS/SIGS directed against plant pathogens that do not possess a canonical RNAi machinery such as the bacterial pathogens that are used heir by the inventors and that do not contain canonical eukaryotic-like RNA silencing factors in their genomes, as explained in the review of S. Ghag, 2017 (22). That is why, as of today, RNA-based silencing technologies have not been exploited to protect plants from bacterial pathogens. This is a considerable limitation because bacterial pathogens have a major impact on agricultural food quality and production, which results in significant economic losses worldwide. This is for instance the case of bacterial pathogens such as Pseudomonas, Ralstonia, Xylella, Xanthomonas, which cause infections of a broad range of cultivated plants (25).

Some authors have speculated that it could be possible to affect bacterial growth by contacting bacterial cells with long dsRNAs. For example, WO 2006/046148 proposes to control the proliferation of pests that can take-up long dsRNA fragments (>80 base pairs), among which, supposedly, bacteria as well. Yet, WO 2006/046148's inventors do not provide any experimental evidence that bacteria are sensitive to such long RNA fragments (their examples only disclose the effect of dsRNAs on nematodes). On the contrary, the present inventors herein demonstrate that bacteria are not sensitive to long dsRNAs, indicating that the hypothesis raised by WO 2006/046148's inventors is not valid when targeting prokaryotic cells.

### Purpose of the Invention

In the present invention, the authors show here for the first time that plant small RNAs can efficiently inhibit the expression of genes from bacterial phytopathogens in a sequence-specific manner, a phenomenon referred to here as “Antibacterial Gene Silencing” (AGS). This regulatory mechanism was notably shown to operate within two different Gram-negative bacterial species, indicating that plant small RNAs can be efficiently taken-up by bacterial cells despite the presence of a cell wall comprising an intricate double membrane structure (the bacterial inner and outer membranes). This is an unexpected result, since it has never been shown in the past that small RNAs can penetrate through the bacterial phospholipid bilayer or be passively or actively transported inside plant pathogenic bacterial cells.

Yet, despite all these prejudices, the Inventors' discoveries demonstrate that it is in fact possible to direct silencing of any bacterial gene, e.g. virulence factors or essential genes, by contacting bacterial cells with small RNAs bearing sequence homologies to one or multiple bacterial target genes. These small RNAs can be stably expressed by said plant cells to protect them against one or multiple bacterial pathogens. Alternatively, they can be exogenously administrated in plant tissues that will encounter the targeted phytopathogenic bacterium, thereby dampening its pathogenicity and growth. Thus, contrary to what was thought so far, small RNA-directed silencing can be used to efficiently knock-down gene expression from plant bacterial pathogens that do not possess an eukaryotic-like RNA silencing machinery and that even possess a double membrane.

This unexpected sensitivity of bacterial cells to exogenously delivered small RNAs can be used purposely in antibacterial applications, and a vast number of treatments can be envisaged to reduce survival, pathogenicity and/or growth of bacterial phytopathogens.

Finally, the Inventors have employed an in vitro-based assay to identify in a rapid, reliable and cost-effective manner, small RNAs with antibacterial activity. Therefore, it is anticipated that the present invention will be extensively employed to (i) confer pre- and post-harvest plant protection against bacterial pathogens, (ii) enhance the beneficial effect and/or the growth of symbiotic or commensal bacteria, and (iii) characterize the function of genes in any bacterial species.

### DETAILED DESCRIPTION OF THE INVENTION

### Overview

In the results below, the Inventors show that AGS is an efficient technology to enhance protection of plants towards bacterial infections by targeting—individually or concomitantly—key genes required for bacterial pathogenicity. They have notably constitutively expressed in Arabidopsis stable transgenic plants, small RNAs bearing homologies to two major virulence factors from the Gram-negative bacterium Pto DC3000, namely Cfa6 and HrpL, and found a significantly lower virulence and growth of this bacterial pathogen when contacted with plant cells expressing these small RNAs. An enhanced protection against Xanthomonas campestris pv. campestris (Xcc), which is the causal agent of black rot, one of the most devastating diseases of crucifer crops, was also observed in Arabidopsis transgenic plants expressing small RNAs against the virulence factors HrpG, HrpX and RsmA. These data demonstrate that AGS can be employed to protect plants against unrelated agriculturally relevant phytopathogens.

They also show that the reduced virulence observed in Arabidopsis transgenic plants expressing anti-Cfa6 and anti-HrpL siRNAs is associated with a specific decrease in the expression of the two-targeted virulence factors in Pto DC3000. This in vivo bacterial gene silencing phenomenon was not only found to be effective against these endogenous stress-responsive virulence genes but also against artificial reporter genes expressed constitutively from the Pto DC3000 genome. These findings therefore highlight that, despite its lack of canonical eukaryotic-like RNA silencing machinery, bacterial cells are actually sensitive to the action of plant-encoded small RNAs. They also provide evidence that artificial small RNAs produced in plants can induce gene silencing in extracellular bacterial pathogens, indicating that the small RNAs must be exported from host cells to bacterial cells, through a mechanism implicating different populations of extracellular plant small RNAs (see below).

Strikingly, this silencing effect has been observed not only on genetically modified plants so as to stably express the small RNAs bearing homology to Cfa6 and HrpL genes, but also on WT plants pre-treated with total RNAs containing anti-Cfa6 and anti-HrpL siRNAs and subsequently inoculated with Pto DC3000. Intriguingly, similar phenotypes were observed when in vitro synthesized anti-Cfa6 and anti-HrpL siRNAs were used in these assays. The latter finding indicates that AGS can not only be triggered by plant-derived small RNAs but also by synthetic double-stranded small RNAs.

In addition, by generating recombinant bacteria expressing a small RNA resilient version of HrpL that contains as many silent mutations as possible in the region that is targeted by small RNAs (which were designed to alter the binding of small RNAs with the HrpL mRNA but to produce the same protein sequence), the Inventors showed that the silencing of HrpL was no longer effective. In addition, they observed that the virulence of this recombinant bacterium remained unaltered upon exogenous application of total RNAs containing effective anti-HrpL small RNAs. These findings provide thus compelling experimental evidence that small RNAs directed against the HrpL gene are causal for both AGS and the dampening of bacterial pathogenicity.

The Inventors went on to further investigate which RNA entities are responsible for the observed AGS phenomenon in response to exogenous total RNAs carrying antibacterial RNAs. Interestingly, by separating small RNA and long RNA species from total RNAs extracted from transgenic plants expressing a chimeric hairpin that target both the Cfa6 and HrpL genes, they showed that the exogenous delivery of the small RNA fraction onto plants triggered the antibacterial effect, while treatment with the long RNA fraction was ineffective. In addition, the inventors showed that total RNA extracts from a IR-CFA6/HRPL reference line, which was mutated in DCL2, DCL3 and DCL4 genes and thus impaired in the biosynthesis of anti-Cfa6 and anti-HrpL siRNAs, were not effective in triggering AGS nor pathogenesis reduction. Collectively, these findings provide compelling evidence that small RNAs, but not their long dsRNA precursors (unless they are processed into small RNAs in planta), are the RNA entities that are causal for AGS. This is a major distinction from environmental RNAi previously reported in C. elegans and plant herbivores, which specifically relies on long dsRNAs (26-32), or in the eukaryotic filamentous pathogens B. cinerea and F. graminearum, which is triggered by either dsRNAs or siRNAs (15, 21).

Importantly, the Inventors additionally demonstrate that exogenous application of total RNAs containing effective small RNAs against the Cfa6 and HrpL genes can efficiently reduce Pto DC3000 growth and pathogenicity in the agriculturally relevant plant Solanum lycopersicum (tomato), which is the natural host of this bacterium. Therefore, it is anticipated that this RNA-based biocontrol approach can be exploited to confer—with a high sequence-based selectivity—protection to a wide range of cultivated plants against bacterial phytopathogens. It can also be predicted that applying small RNAs bearing sequence homologies to virulence factors and/or essential genes on the surface of fruits, vegetables, flowers and leaves will significantly reduce infection against various devastating bacterial pathogens. This approach would also be suitable in seed treatments to control seed-borne bacterial pathogens, which represent major threat for the seed industry. Furthermore, this method can be easily designed to control multiple bacterial pathogens by concomitantly targeting essential genes and/or virulence factors from various bacterial phytopathogens. AGS therefore represents a novel environmental friendly RNA-based technology to protect plants against bacterial diseases.

In the past, Escobar et al (33) has used an RNA silencing-based approach to target the two Agrobacterium tumefaciens oncogenes iiaM and ipt in both Arabidopsis and tomato. However, in this report the authors have targeted oncogenes that are transferred and integrated within the plant genome by the T-DNA from the large tumor-inducing (Ti) plasmid. As a matter of fact, the mechanism that is manifested relies on a classical RNA silencing process of targeted genes that are expressed from plant cells but not from bacterial cells. This is a major distinction from the present invention that relies instead on the silencing of genes that are expressed within the bacterial cells. Furthermore, in Escobar et al (33) study, only the crown gall tumorigenesis induced by the Agrobacterium tumefaciens bacteria is prevented, because the RNA silencing effect inhibits the expression of the oncogenes iiaM and ipt expressed from plant cells. Therefore, the results can be only transposed to other tumorigenesis-inducing bacteria that are known to integrate their DNA into the plant genome. Consequently, this study has a limited impact on plant protection, as highlighted in the study itself (page 13442, right column; “Good nursery practices would need to be maintained in the cultivation of these plants to minimize the dissemination of large populations of virulent Agrobacterium tumefaciens bacteria into the field”). As a matter of fact, the intrinsic virulence of these bacteria was not affected by the small RNAs used by the authors in this study.

In the results below, the inventors have also investigated the possible role of EVs in the trafficking of plant-derived antibacterial small RNAs towards bacterial cells. They have discovered at least two populations of EVs possessing antibacterial activities, one of large size, which were fully active in dampening bacterial pathogenesis, and another one of smaller EVs, which were moderately less active. Furthermore, they showed that antibacterial small RNAs are protected from micrococcal nuclease (Mnase) digestion when embedded within these EVs, highlighting the potential of plant EVs for future disease management strategies in field conditions. Intriguingly, the inventors have additionally discovered that apoplastic EV-free antibacterial small RNAs, which were not associated with proteins, were also fully active in dampening pathogenesis. These novel small RNA species are referred to here as Extracellular Free Small RNAs or “efsRNAs”, and were sensitive to Mnase digestion. The inventors therefore concluded that the apoplast of IR-CFA6/HRPL transgenic plants is composed of at least three populations of functional antibacterial small RNAs, which are either embedded in large EVs, in smaller EVs, or in a free form.

The Inventors have also transiently expressed small RNAs using well-established Agrobacterium-mediated transient transformation of tobacco leaves, followed by the incubation of corresponding candidate antibacterial siRNAs with bacterial cells. This approach was notably useful to determine that siRNAs directed against the HrpL gene were equally efficient in preventing Pto DC3000-induced stomatal reopening as compared to siRNAs targeting Cfa6 and HrpL genes concomitantly. Furthermore, the inventors have demonstrated that the in vitro synthesis of small RNAs is an easy, rapid and reliable approach to screen for candidate small RNAs triggering antibacterial effects, such as bacterial gene silencing and the suppression of bacterial-induced stomatal reopening. They have also coupled the in vitro small RNA synthesis approach with a droplet-based microfluidic system to demonstrate that siRNAs directed against the conserved genes GyrB or FusA from Pto DC3000 can drastically alter bacterial growth in vitro, thereby identifying novel bactericidal agents. It is therefore anticipated that such transient tobacco- or in vitro-based synthesis of candidate small RNAs, followed by incubation of corresponding small RNAs with bacterial cells in vitro, will be extensively employed in the future by industrials to identify small RNAs having strong effects on bacterial gene expression and/or on specific phenotypes (e.g. bacterial growth, survival, metabolic activities). It is also anticipated that the AGS technology described herein will be widely used to characterize the function of bacterial genes through a novel RNA-based reverse genetic approach. This method was for instance instrumental to demonstrate for the first time a role for HrpL in Pto DC3000-induced stomatal reopening, as well as a role for GyrB and FusA in the survival of Pto DC3000. Finally, because tobacco plants are already used by industrials to produce high yields of recombinant proteins or vesicle-like particles in a cost-effective manner (cf. EP2610345 from Medicago Inc.), they will likely be exploited to produce candidate small RNAs, particularly within EVs in which they will be protected from nuclease degradation, for future RNA-based biocontrol applications.

Based on all these discoveries, the present Inventors propose a method to inhibit the expression of at least one gene in bacteria, said method comprising either:

i) introducing into at least one plant cell at least one functional interfering RNA molecule (iRNA) targeting specifically at least one bacterial gene, said iRNA being able to induce sequence-specific silencing of said gene(s) in bacteria carrying said gene(s), or
ii) delivering small RNAs or plant extracts containing these small RNAs on plant tissues prior to and/or after bacterial infection, or
iii) delivering small RNAs directly on bacterial cells, these small RNAs being in EVs, in apoplastic fluids or as extracellular free RNAs.

Interestingly, this method allows the targeting of one or multiple bacterial gene(s) by expressing interfering RNA molecules (precursors of siRNAs and miRNAs) in plant cells or delivering small RNAs on plant tissues prior to and/or after bacterial infection. This approach will have major agricultural, RNA-based biocontrol applications in the management of bacterial infections in plants.

More precisely, this technology will provide a way to control bacterial infections in plants, and therefore reduce chemical treatments in agriculture without having a negative effect on beneficial bacteria or on the environment due to the high sequence-based selectivity of this approach.

Furthermore, this strategy will provide durable disease resistance, which is not the case with conventional or molecular breeding programs that aim to introduce a single disease resistance gene in crops.

Finally, it can be anticipated that the herein described technology will also be useful to control the expression of genes from beneficial bacteria in order to enhance their multiplication and/or their beneficial effects for the host plants.

Besides these advantages, the proposed method is cost-effective and relatively easy to industrialize. Indeed, the process of designing and producing effective artificial iRNAs (such as siRNAs) against bacterial genes only takes a few weeks when transiently expressed from N. benthamiana leaves or even a single day when synthesized in vitro. Furthermore, it is relatively easy to redesign and produce de novo artificial iRNAs upon appearance of siRNA-resistant bacteria. Finally, it is possible to produce iRNAs directed against either a specific bacterial species or against a vast range of phytopathogenic bacterial strains thereby providing targeted or broad-spectrum plant treatments depending on the RNA-based biocontrol application desired.

The present method/use can be performed either in vivo or in vitro. By “in vitro”, it is herein meant that the steps of the claimed methods or uses are conducted using biological components (e.g., bacterial cells) that have been isolated from their usual host organisms (plants or plant tissues such as leaves, fruits, roots, etc.) or that are directly grown in in vitro media (in the absence of their host organisms). This is the case when the small RNAs of the invention are contacted directly with the bacterial cells.

By “in vivo” or “in planta”, it is herein meant that the steps of the claimed methods or uses are conducted using whole organisms, for example whole plants. This is the case when the iRNAs of the invention are stably expressed from plants, where they are processed into small RNAs (siRNAs or miRNAs) and then released into the surrounding medium so as to encounter the bacterial cells.

When small RNAs of the invention are contacted directly with bacterial cells, notably to trigger gene silencing of virulence factors within bacterial cells, the present method/use is said to be performed in a “semi-in vivo” assay.

It is noteworthy that all the methods of the invention are to be performed outside animal or human organisms.

### RNA Silencing Elements

The present invention targets the use of at least one functional interfering RNA (iRNA) for inhibiting the expression of at least one gene in a bacterial cell.

As used herein, the term “functional interfering RNA” (functional iRNA) refers to a RNA molecule capable of inducing the process of sequence-specific silencing of at least one bacterial gene(s), especially in bacteria cells. In particular, said functional interfering RNA molecule can be either i) a small interfering RNA, well-known in the art as small or short interfering RNA (siRNA) molecule (simplex or duplex) or a precursor thereof, or ii) a microRNA (miRNA) molecule (simplex or duplex) or a precursor thereof.

The term “precursor of siRNA” or “siRNA precursor” herein refers to an RNA molecule which can be directly or indirectly processed into siRNA duplex(es) in plants (or plant extracts). Examples of siRNA precursors that can be directly processed include long double-stranded RNA (long dsRNA), while examples of siRNA precursors that can be indirectly processed include long single-stranded RNA (long ssRNA) that can be used as template for the production of processable long dsRNAs.

The term “precursor of miRNA” or “miRNA precursor” herein refers to an RNA molecule which can be processed into miRNA duplex(es) in plants (or plant extracts). Examples of miRNA precursors include primary miRNA precursors (pri-miRNAs) and pre-miRNAs, comprising a hairpin loop.

Of note, plasmids or vectors and other DNA constructs or viral vectors encoding said precursor molecules are also encompassed in the definition of “functional interfering iRNA”.

For targeting multiple genes in a bacterium, the method of the invention can use i) a mixture of several different iRNAs which altogether target multiple bacterial genes of interest or ii) a chimeric iRNA targeting several different bacterial genes of interest or iii) a mixture of any of these chimeric iRNAs.

In one particular embodiment, the method/use of the invention comprises the introduction of one or several functional iRNAs into plant cells as precursors, to produce in planta the small RNAs (such as siRNAs or miRNAs) that can be further formulated and sprayed in a plant field.

In a more particular embodiment, the functional iRNAs of the invention are long single-stranded RNA molecules (named hereafter as “long ssRNAs”). Such long ssRNA may be produced by a plant transgene, converted into long dsRNA molecules by plant RNA-dependent RNA polymerases, and further processed into siRNAs by plant DCL proteins.

Alternatively, long ssRNA may be produced by a plant RNA virus and further converted into long dsRNA molecules either during viral replication (as replicative intermediates) and/or through the action of plant RNA-dependent RNA polymerases. The resulting viral dsRNA is subsequently processed into siRNAs by plant DCL proteins, which subsequently trigger sequence-specific silencing through a process referred to as Virus-Induced Gene Silencing (VIGS) (11).

As used herein, the term “long ssRNA” designates single-stranded structures containing a single strand of at least 50 bases, more preferably of 80 to 7000 bases. Long ssRNAs may contain 80 to 7000 bases when produced by a plant transgene, but preferably contain 80 to 2000 bases when produced by a plant recombinant RNA virus.

In a more particular embodiment, the functional iRNAs of the invention are long double-stranded RNA molecules (named hereafter as “long dsRNAs”) that act as siRNA precursor and can be processed into siRNAs, in planta, thanks to DCL proteins and other small RNA biogenesis factors encoded by plant genomes.

As used herein, the term “long dsRNA” designates double-stranded structures containing a first (sense strand) and a second (antisense) strand of at least 50 base pairs, more preferably of 80 to 7000 base pairs.

In plants or plant cells, long dsRNAs can be processed into small RNA duplexes. Such long dsRNAs are advantageously chimeric dsRNA, i.e., they bear sequence homologies to multiple bacterial genes (see below).

In one embodiment, the functional iRNA of the invention is a long dsRNA that is cleavable by DCL proteins in plant cells so as to generate siRNAs.

The long dsRNAs of the invention can be generated from a hairpin structure, through sense-antisense transcription constructs, through an artificial sense transcript construct further used as a substrate by plant RNA-dependent RNA polymerases, or through VIGS. More precisely, they may comprise bulges, loops or wobble base pairs to modulate the activity of the dsRNA molecule so as to mediate efficient RNA interference in bacterial cells. The complementary sense and antisense regions of the long dsRNA molecule of the invention may be connected by means of nucleic acid based or non-nucleic acid based linker(s). The long dsRNA of the invention may also comprise one duplex structure and one loop structure to form a symmetric or asymmetric hairpin secondary structure.

Therefore, in one embodiment, the functional iRNA of the invention is a long (at least 50 base pairs, more preferably of 80 to 400 base pairs, 100 to 200 base pairs, 125 to 175 base pairs, in particular about 150 base pairs) dsRNA comprising a hairpin such as miRNA precursors.

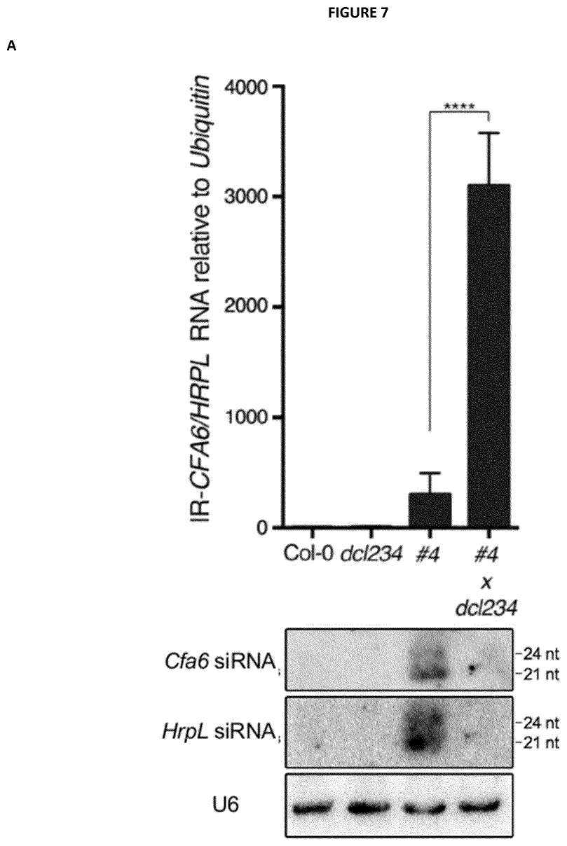

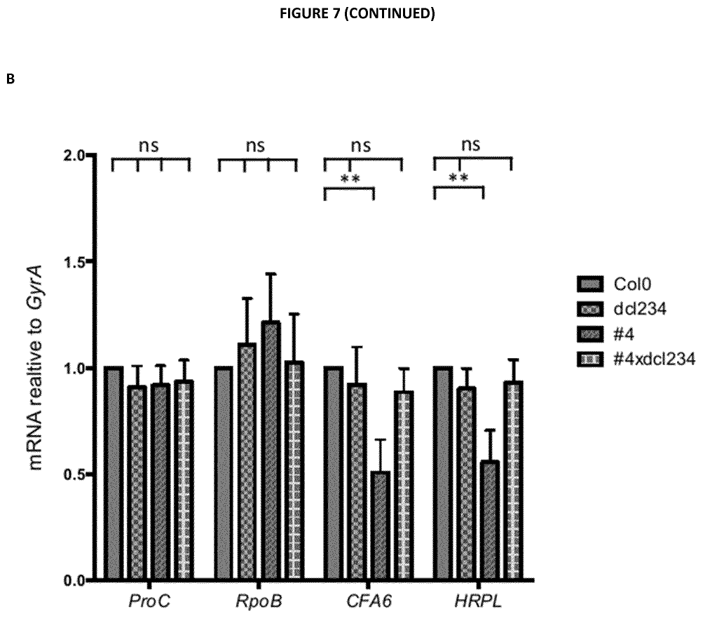

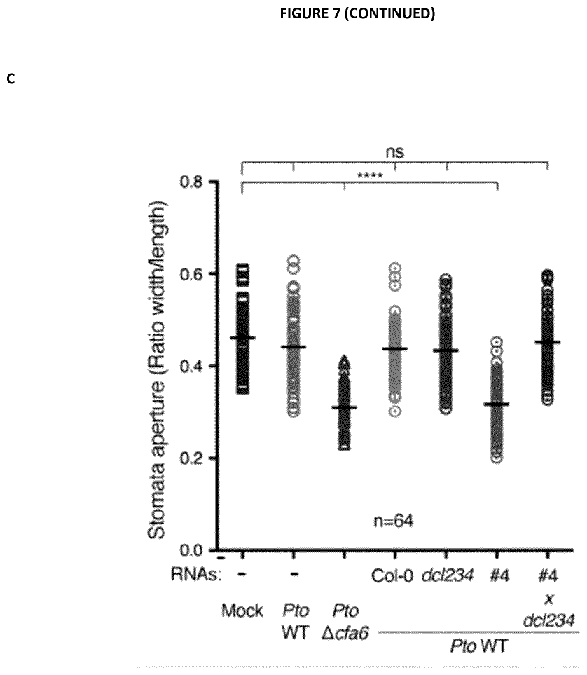

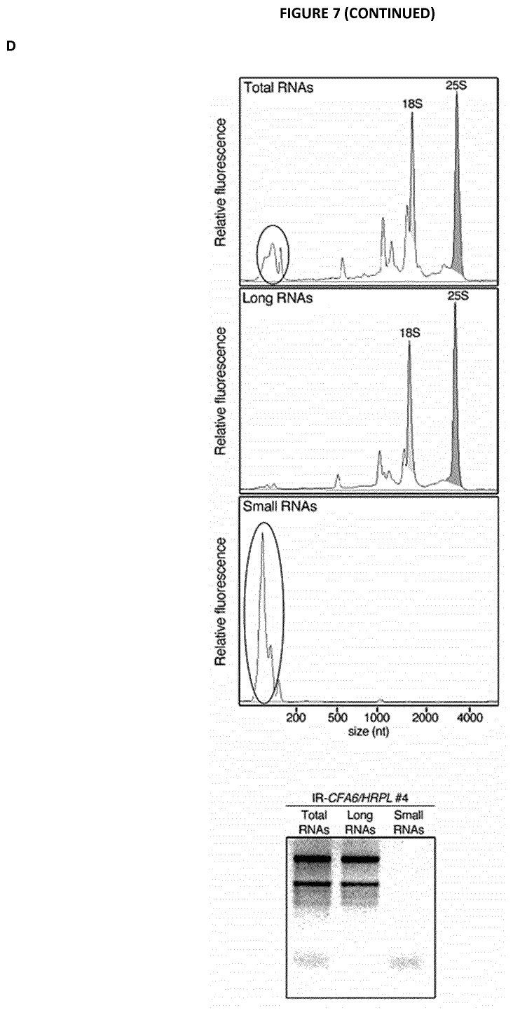

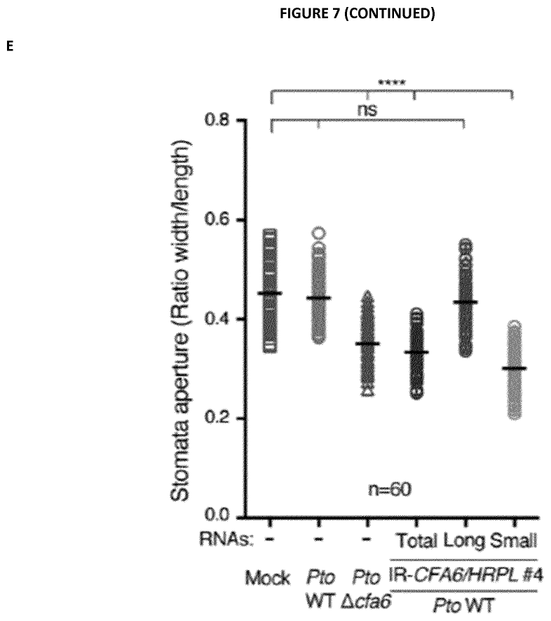

As demonstrated in the examples of the present application, the introduction of dsRNA into plant cells induces a sequence-specific silencing of the bacterial gene(s) in the bacteria cells through the action of small RNAs but not long dsRNAs (example 6 & FIG. 7). This means that bacterial cells are only sensitive to AGS when they are directly contacted by small RNA entities. Contacting bacteria with precursors of small RNAs will have no effect since these prokaryotic cells do not possess the canonical eukaryotic-like machinery to process them properly into functional iRNAs. This has notably been shown by inactivating Arabidopsis DCL2, DCL3 and DCL4 genes in a reference transgenic IR-CFA6/HRPL #4 line. Total RNAs derived from these plants expressed abundant inverted repeat CFA6/HRPL transcripts (i.e. unprocessed CFA6/HRPL dsRNAs) that were not competent in triggering AGS of Pto DC3000 Cfa6 and HrpL genes nor pathogenesis reduction (FIG. 7).

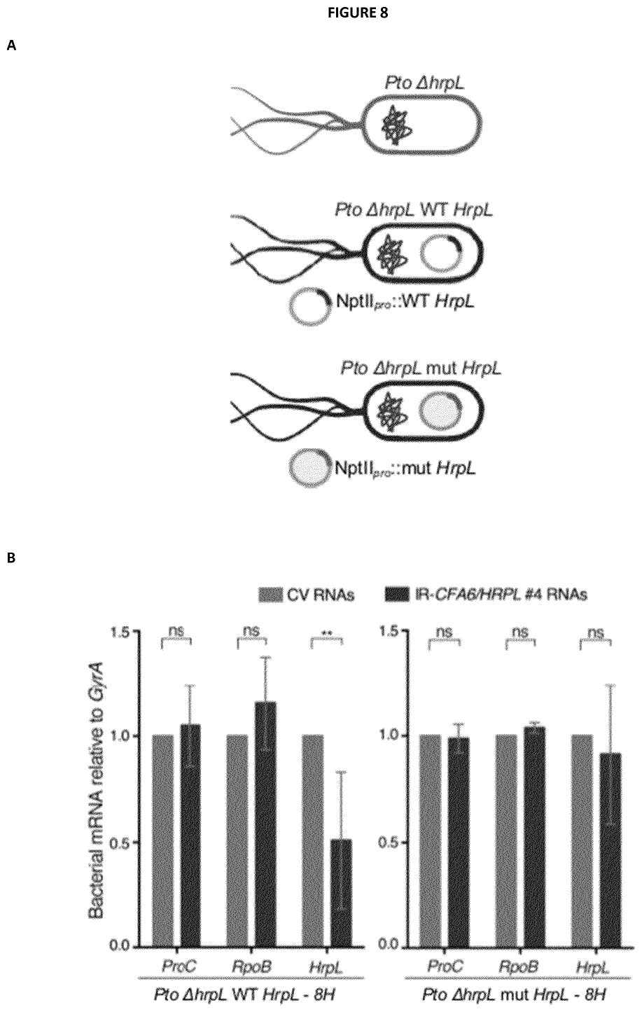

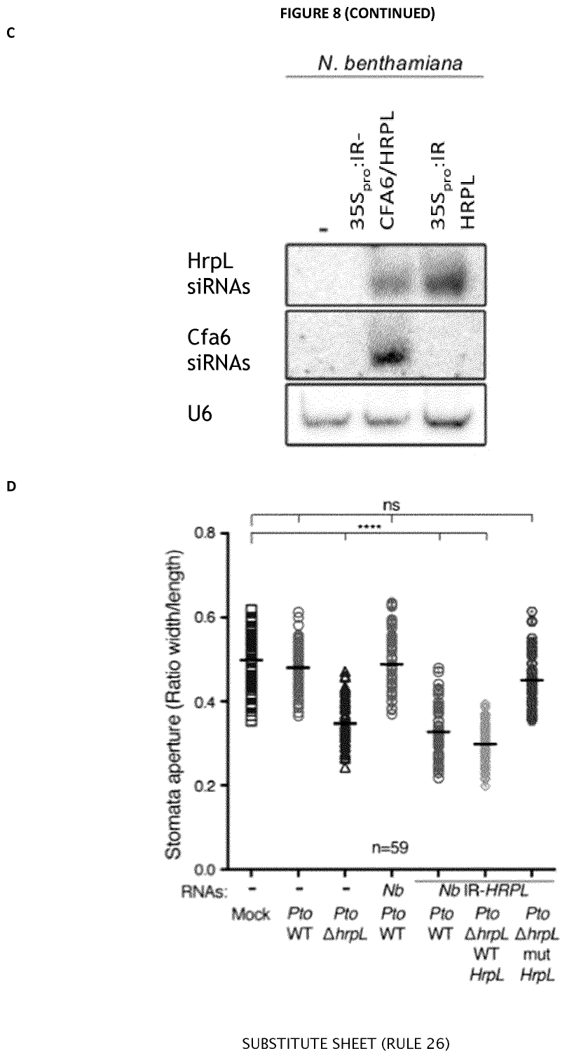

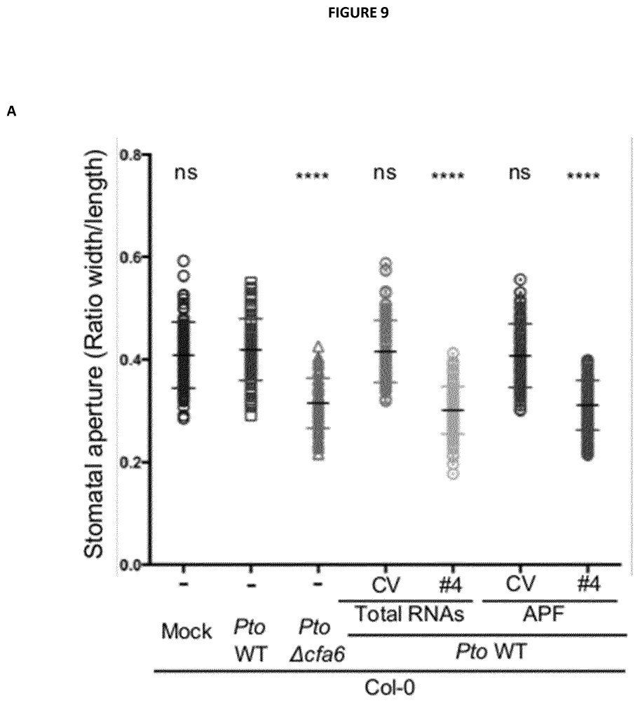

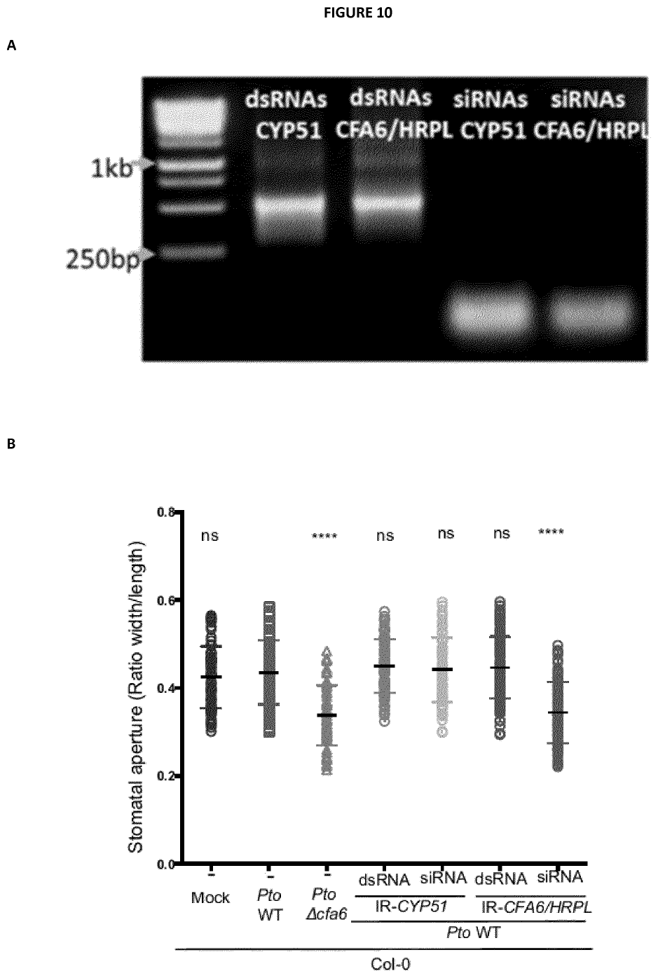

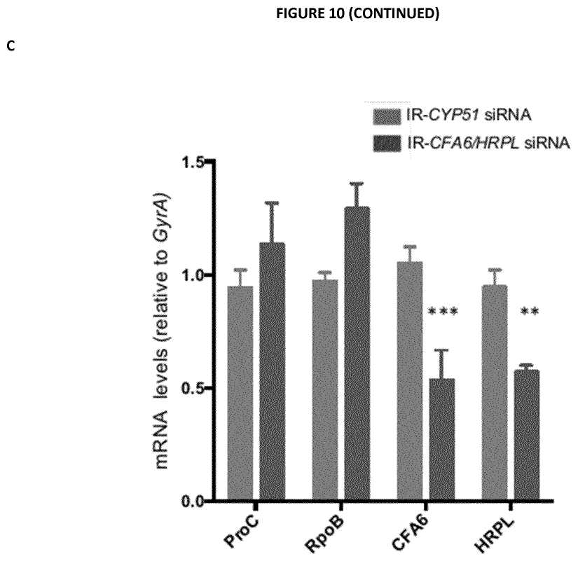

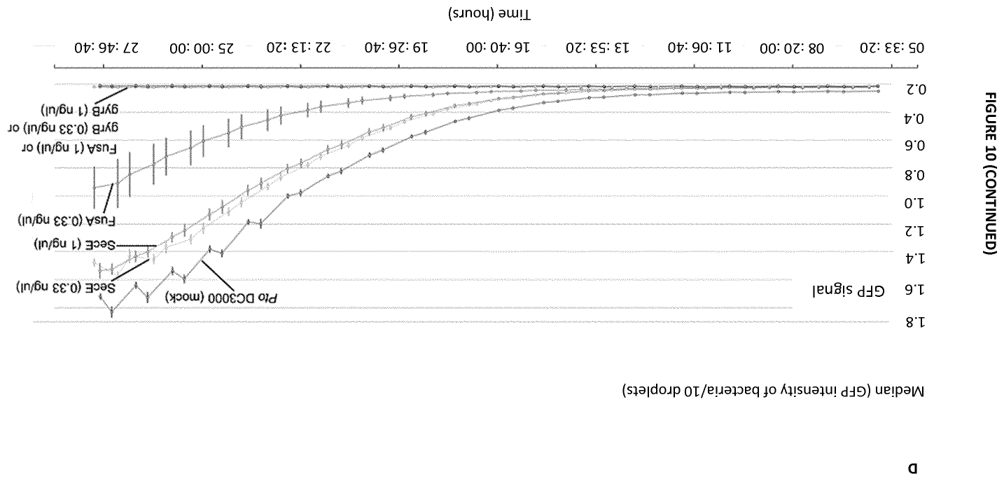

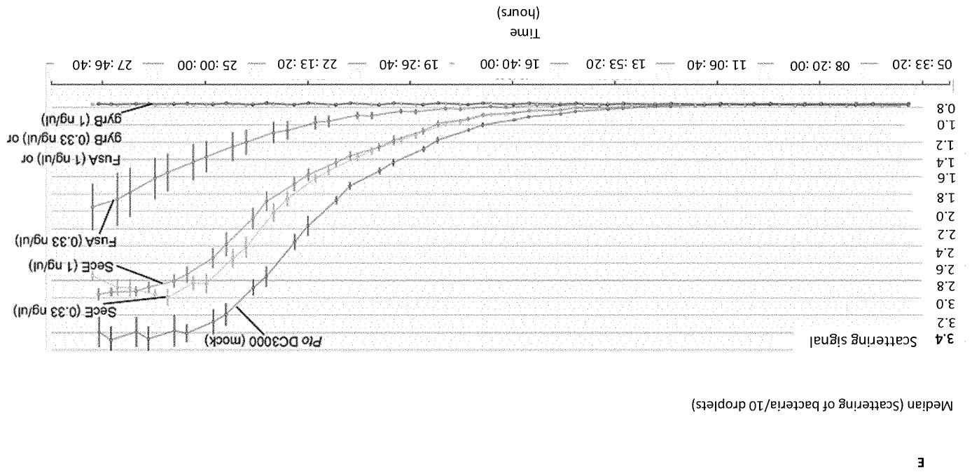

Nevertheless, it is possible to inhibit the expression of bacterial genes directly in bacterial cells by contacting them with small RNA species whose size is shorter than 50 base pairs (FIG. 8 & FIG. 10).

Therefore, in another preferred embodiment, the functional iRNAs of the invention are small RNAs such as siRNAs or miRNAs. These small RNAs have a short size, which is less than 50 base pairs, preferably comprised between 15 and 30 base pairs, more preferably between 19 and 27 base pairs, even more preferably between 20 and 25 base pairs.

These small RNAs can be formulated in phytosanitary compositions, e.g., into sprayable liquid compositions (see below). In this case, the said compositions containing the said small RNAs can be administered directly to plant tissues or to bacteria.

In one particularly preferred embodiment, the functional iRNA of the invention is a “siRNA”, which designates either a “siRNA duplex” or a “siRNA simplex”.

More specifically, the term “siRNA duplex” designates double-stranded structures or duplex molecules containing a first (sense strand) and a second (antisense) strand of at least 15 base pairs, preferably of at least 19 base pairs; preferably, said antisense strand comprises a region of at least 15 contiguous nucleotides that are complementary to a transcript of the targeted gene. These siRNA duplexes are produced from long dsRNA precursors that are processed by plant DCL proteins. They have a short size which is less than 50 base pairs, preferably comprised between 15 and 30 base pairs, more preferably between 19 and 27 base pairs, even more preferably between 20 and 25 base pairs.

As shown in the experimental part below (Example 9 and FIG. 10), the small RNAs of the invention are efficient when they are under double-stranded structure. It has been demonstrated with in vitro de novo synthesized siRNA duplexes, and it is thought that the biological effect observed with plant extracts is at least in part due to these siRNA duplexes secreted by the plants.

As used herein, the term “siRNA simplex” or “mature siRNA” designates simplex molecules (also known as “single-stranded” molecules) that originate from the siRNA duplex but have been matured in the RISC machinery of a plant cell and are loaded in an AGO protein and/or associated with other RNA-binding proteins. They have a short size which is less than 50 bases, preferably comprised between 15 and 30 bases, more preferably between 19 and 27 bases, even more preferably between 20 and 25 bases.

In another embodiment, the functional iRNA of the invention is a “miRNA”, which designates either a “miRNA duplex” or a “miRNA simplex”.

More specifically, the term “miRNA duplex” designates double-stranded structures or duplex molecules containing a first (sense strand) and a second (antisense) strand of at least 15 base pairs, preferably of at least 19 base pairs; preferably, said antisense strand comprises a region of at least 15 contiguous nucleotides that are complementary to a transcript of the targeted gene. These miRNA duplexes may also contain bulges. These miRNA duplexes are produced from miRNA precursors that are processed by plant DCL proteins.

As used herein, the term “miRNA simplex” or “mature miRNA” designates simplex molecules (also known as “single-stranded” molecules) that originate from the miRNA duplex but have been matured in the RISC machinery of a plant cell and are loaded in an AGO protein and/or associated with other RNA-binding proteins.

Methods to design iRNAs such as long dsRNAs/siRNAs/miRNAs are available in the art and can be used to obtain the sequence of long dsRNAs, siRNA and miRNA having these properties.

The inventors herein show (Example 9, FIG. 10) that it is possible to use artificial in vitro synthetized double-stranded siRNAs in order to (i) inhibit bacterial gene expression (ii) dampen bacterial pathogenicity, and (iii) trigger bactericidal effects in vitro (see FIG. 10).

The invention therefore encompasses the use of synthetic, semi-synthetic or recombinant iRNAs comprising ribonucleotides only or both deoxyribonucleotides and ribonucleotides. The invention also encompasses the use of modified iRNA molecules comprising one or more modifications, which increase resistance to nuclease degradation in vivo and/or improve cellular stability (e.g. small RNA 3′ end methylation, locked nucleic acid (LNA)), uptake by bacterial cells (e.g. peptide carriers) or silencing efficacy within bacterial cells. The iRNAs of the invention may include nucleotides, which are modified at the sugar, phosphate, and/or base moiety, and/or modifications of the 5′ or 3′ end(s), or the inter-nucleotidic linkage.

Chemically synthesized dsRNA molecules as defined in the invention may be assembled from two distinct oligonucleotides, which are synthesized separately. Alternatively, both strands of the RNA duplex or RNA precursor molecule may be synthesized in tandem using a cleavable linker, for example a succinyl-based linker. Alternatively, the RNA precursor molecules of the invention may be expressed (in vitro or in planta) from transcription units inserted into DNA or RNA vectors known to those skilled in the art and commercially available. It is noteworthy that the latter approach can include the transcription of transgenes expressing long double-stranded fold-back structures, sense-antisense transcripts through promoters positioned on both ends of the transgene and in opposite orientation, miRNA precursors, primary miRNA transcript, or sense transcripts that can, in some instances (e.g. targeted by endogenous or exogenous 22 nt long miRNAs) be used as substrates by the plant RNA-dependent RNA polymerases to generate dsRNAs.

The iRNA molecules of the invention preferably decrease the level of expression of the targeted bacterial gene(s) by at least 30%, preferably by at least 60%, more preferably by at least 80%, in bacteria carrying said gene(s). The silencing of the bacterial gene(s) can be assessed at the RNA or protein level, by methods well-known in the art, for example by Real time quantitative RT-PCR (RT-qPCR), Northern Blot, FACS, Immunohistological analyses or Western Blot analyses.

In the context of the invention, the silencing of the bacterial gene(s) by artificial iRNA molecules, which may be partial or total, should be sufficient to produce the desired effect on the bacteria, such as for example to reduce bacterial pathogenicity or infectivity of said bacteria in plant cells or in the plant organism.

### Targeted Bacteria

The use/method of the invention is useful for silencing genes in any type of bacteria (pathogenic or non-pathogenic; Gram-positive or Gram-negative), including beneficial bacteria known to be associated with plant organisms.

Non-limitative examples of pathogenic bacteria which can be targeted using the use/method of the invention include Ralstonia solanacearum, Xanthomonas oryzae pathovars, Xanthomonas campestris pathovars, Xanthomonas axonopodis pathovars, Xanthomonas euvesicatoria pathovars, Xanthomonas hostorum pathovars, Pseudomonas syringae pathovars, Pseudomonas viridiflava pathovars, Pseudomonas savastonoi pathovars, Candidatus liberibacter asiaticus, Candidatus liberibacter solanacearum, Acidovorax citrulli, Acidovorax avenae pathovars, Pectobacterium atrosepticum pathovars, Pectobacterium carotovorum pathovars, Pectobacterium sp., Agrobacterium tumefaciens, Dickeya (dadantii and solani), Erwinia amylovora, Clavibacter michiganensis (michiganensis and sepedonicus), Xylella fastidiosa, Pectobacterium (carotovorum and atrosepticum), Streptomyces scabies, Phytoplasma sp., Spiroplasma sp. (25).

If ornamental plants are treated, the following bacteria can be targeted: Pseudomonas cichorii, known to infect Chrysanthemum, Geranium, and Impatiens; Xanthomonas campestris pv. pelargoni, known to infect Geranium; Rhodococcus fascians, known to infect Chrysanthemum morifolium, Pelargonium, Phlox, and possibly Rhododendron; Ralstonia solanacearum, known to infect Geranium, Anthurium spp, Rose tree, Curcumas, and Anthuriums; Xanthomonas axonopodis, Xanthomonas hortorum, known to infect Geranium, Begonia, Anthurium, and Hibiscus rosa-sinensis; Pectobacterium carotovorum, known to infect Amaryllis, Begonia, Calla, Cyclamen, Dracena and Impatiens.

In a particular embodiment, the method of the invention uses functional iRNA(s) targeting one or multiple genes of beneficial bacteria often referred to as Plant-growth-promoting rhizobacteria (PGPR). The purpose of this particular embodiment is to promote the beneficial effects of said PGPR. In this particular embodiment, the targeted bacterial genes are factors that, when silenced, promote the replication of the targeted bacterial cells or a pathway that is beneficial for the host and that positively regulate the production of a beneficial compound (e.g. a phytohormone), secondary metabolites that (i) alter the survival/pathogenicity of surrounding phytopathogens, (ii) activate plant defense responses (e.g. Induced Systemic Resistance), (iii) facilitate the uptake of nutrients from the environment (e.g. by enhancing the production of bacterial factors that are essential for Rhizobium-legume symbiosis), (iv) enhance the tolerance of the host organism to abiotic stress conditions etc. Silencing of such bacterial targeted genes would thus lead to an increased growth rate of the host organism and/or several other possible beneficial effects for the host organism.

In such an embodiment, the iRNAs of the invention should have sequence homologies with beneficial bacterial genes but no sequence homology to pathogenic bacterial genomes, with the host genome or with other genomes of host colonizers and/or mammals that feed on the host organism.

Non-limitative examples of beneficial bacteria which can be targeted with the method of the invention include: Bacillus (e.g. Bacillus subtilis), Pseudomonas (e.g. Pseudomonas putida, Pseudomonas stuzeri, Pseudomonas fluorescens, Pseudomonas protegees, Pseudomonas brassicacearum), Rhizobia (Rhizobium meliloti), Burkholderia (e.g. Burkholderia phytofirmans), Azospirillum (e.g. Azospirillum lipoferum), Gluconacetobacter (e.g. Gluconacetobacter diazotrophicus), Serratia (e.g. Serratia proteamaculans), Stenotrophomonas (e.g. Stenotrophomonas maltophilia), Enterobacter (e.g. Enterobacter cloacae).

### Targeted Bacterial Genes

The iRNA of the invention should have a sufficient sequence homology with at least one bacterial gene in order to induce sequence-specific silencing of said at least one gene. In addition, to prevent unwanted off-target effects, the sequence homology of the dsRNAs, miRNAs or small RNA species of the invention with the eukaryotic host genome or other genomes of beneficial bacteria, host colonizers and/or mammals that feed on the host organism should be quasi inexistent (if not absent).

The iRNA of the invention is able to inhibit the expression of at least one bacterial gene.

According to the invention, the term “bacterial gene” refers to any gene in bacteria including (natural) protein-coding genes or non-coding genes, present naturally in bacteria or artificial genes introduced in bacteria by recombinant DNA technology. Said target bacterial genes are either specific to a given bacterial species or conserved across multiple bacterial species. Preferably, it shares no homology with any gene of the eukaryotic host genome, host colonizers and/or mammals that feed on the host organism. This avoids collateral effects on the plant host, beneficial bacteria associated with the host, host colonizers and/or mammals that feed on the host organism.

In a preferred embodiment, said at least one bacterial gene is a bacterial virulence factor or an essential gene for bacteria.

As used herein, the term “essential gene for bacteria” refers to any bacterial gene that is essential for bacterial cell viability. These genes are absolutely required to maintain bacteria alive, provided that all nutrients are available. It is thought that the absolutely required number of essential genes for bacteria is about 250-500 in number. The identification of such essential genes from unrelated bacteria is now becoming relatively easily accessible through the use of transposon sequencing approaches. These essential genes encode proteins to maintain a central metabolism, replicate DNA, ensure proper cell division and/or elongation, translate genes into proteins, maintain a basic cellular structure, and mediate transport processes into and out of the cell (34). This is the case of GyrB and FusA, whose silencing were found to drastically impair the growth of Pto DC3000 in vitro (FIGS. 10D and 10E).

As used therein, the term “virulence gene” refers to any bacterial gene that has been shown to play a critical role for at least one of the following activity: pathogenicity, disease development, colonization of a specific host tissues (e.g. vascular tissues) or host cell environment (e.g. the apoplast), suppression of PTI and ETI responses, modulation of plant hormone signaling and/or biosynthesis to facilitate multiplication and/or disease development, interference with conserved host regulatory processes to facilitate multiplication and/or disease development, etc. All these activities help the bacteria to grow and/or promote disease symptoms in the host, although they are not essential for their survival in vitro. Well-known virulence factors are: adhesins, phytotoxins (e.g. coronatine, syringoline A), degradating enzymes (e.g. cellulases, cellobiosidases, lipases, xylanases, endoglucanases, polygalacturonases), factors required for the assembly of type I/II/III/IV or VI secretion systems, effector proteins, transcription factors required to promote the expression of Hrp genes upon contact with plant cells, machineries required for the proper expression of virulence factors (e.g. quorum sensing, two-component systems), post-transcriptional factors controlling the stability/translation of mRNAs from virulence factor genes.

Well-known bacterial essential genes or virulence factors are provided in the following tables, for several different phytopathogenic bacteria:
For most bacteria: central factors required for cell division such as FtsZ, FtsA, FtsN, FtsK, FtsI, FtsW, ZipA, ZapA, TolA, TolB, ToiQ, ToiR, Pal, MinCD or actin-related genes such as MreB and Mld.
For the bacteria Pseudomonas syringae pv. phaseolicola (strains Pph 1448A; Pph 1302A), known to infect common bean plants Phaseolus vulgaris and cause the halo blight disease:

| Essential | | |
|---|---|---|
| gene | Description | Function |
| cheA2 | Chemotaxis sensor histidine kinase | Chemotaxis |
| | CheA | |
| cheZ | Chemotaxis protein CheZ | Chemotaxis |
| flhB | Flagellar biosynthetic protein FlhB | Flagellum |
| fliO | Flagellar protein FliO | Flagellum |
| fliN | Flagellar motor switch protein FliN | Flagellum |
| fliM | Flagellar motor switch protein FliM | Flagellum |
| fliK | Flagellar hook-length control protein | Flagellum |
| | FliK | |
| fleS | Flagellar sensor histidine kinase FleS | Flagellum |
| flgJ | Peptidoglycan hydrolase FlgJ | Flagellum |
| flgE | Flagellar hook protein FlgE | Flagellum |
| flgD | Basal-body rod modification protein | Flagellum |
| | FlgD | |
| FlgA | Flagellar basal-body P-ring formation | Flagellum |
| | protein, putative | |
| MotA | MotA family motility protein | Flagellum |
| plsC | hdtS protein (100%) | quorum-sensing |
| | | molecule |
| impL | OmpA domain protein (98%) | Outer membrane |
| | | protein and |
| | | pathogenicity; |
| | | might |
| | | be involved |
| | | in T6SS |
| mdoG1 | periplasmic glucans biosynthesis | |
| | protein MdoG | |
| CobQ | cobyric acid synthase | cobalamin |
| | | (vitamin B12) |
| | | biosynthesis |
| carA | carbamoyl-phosphate synthase, | pyrimidine |
| | small subunit | biosynthesis |
| pyrB | aspartate carbamoyltransferase | pyrimidine |
| | | biosynthesis |
| carB | carbamoyl-phosphate synthase, | pyrimidine |
| | large subunit | biosynthesis |
| purH | bifunctional purine biosynthesis | purine |
| | protein PurH | biosynthesis |
| pyrD | dihydroorotate dehydrogenase | pyrimidine |
| | cobalamin synthesis protein/P47K | biosynthesis |
| | family protein | |

For the bacteria Pseudomonas syringae pv. actinidiae, known to infect kiwi plants and fruits (Actinidia spp.) and cause the kiwifruit canker:

| Essential gene | Description |
|---|---|
| fliR | Flagellar biosynthesis protein |
| flhA | Flagellar biosynthesis protein |
| flgC | Flagellar basal-body rod protein |
| flgH | Flagellar biosynthesis protein |
| FlgL | flagellar hook-associated protein |
| | Flagellar basal-body rod |
| | modification protein |

For the bacteria Pectobacterium carotovorum, known to infect the Chinese cabbage and cause the soft rot disease:

| Essential gene | Description |
|---|---|
| pyrD | Dihydroorotate dehydrogenase |
| purH | Bifunctional phosphoribosylamino- |
| | imidazole carboxamide formyltransferase/IMP |
| | cyclohydrolase |
| purD | Phosphoribosylamine-glycine ligase |
| leuA | 2-Isopropylmalate synthase |
| serB | Phosphoserine phosphatase |
| expl | Synthesis of N-(3-oxohexanoyl)-I-homoserine |
| | lactone |
| expR | Quorum-sensing transcriptional regulator |
| flgA | Flagellar basal body P-ring biosynthesis protein |
| fliA | Flagellar biosynthesis sigma factor |
| flhB | Flagellar biosynthesis protein |
| qseC | Sensor protein QseC bacterial adrenergic receptor |
| tolC | Outer membrane channel protein |
| PCC21_023220 | Putative DNA-binding protein |

For the bacteria Ralstonia solanacearum, known to infect for instance tomato, potato, tobacco, banana and soybean and cause the bacterial wilt:

| | |
|---|---|
| Essential gene | Description |

in Tomato

| | |
|---|---|
| RSc2735 (phcB) | Regulatory protein |
| RSc2748 (phcA) | A LysR family transcriptional regulator, |
| | virulence genes transcriptional regulator |
| RSp0852 (hrpG) | Transcription regulator protein |
| RSc2827 (pilD) | Probable type 4 prepilin peptidase; |
| | bifunctional: leader peptidase and N-methyl- |
| | transferase transmembrane protein |
| RSc3111 (gspL) | Probable general secretory pathway L |
| | transmembrane protein |
| RSc3114 (gspD) | Probable general secretory pathway D |
| | transmembrane protein |
| RSp1007 | Putative acetyl transferase protein |
| RSc0571 (pgk) | Probable phosphoglycerate kinase protein |
| RSc1591 (hpal1) | Putative 2,4-dihydroxyhept-2-ene-1,7- |
| | dioic acid aldolase oxidoreductase protein |
| RSp1010 | hypothetical protein |
| RSc3115 (gspE) | Probable general secretion pathway protein E |

in Arabidopsis

| | |
|---|---|
| RSc0903 (serC) | Probable phosphoserine aminotransferase |
| | (PSAT) protein |
| RSc1981 (trpA) | Probable tryptophane synthase alpha chain |
| | protein |
| RSc0454 | Putative Fe—S oxidoreductase; FAD/FMN- |
| | containing dehydrogenase oxidoreductase protein |
| RSc0411 | Probable transmembrane protein |
| RSc0353 (gpmA) | Probable 2,3-bisphosphoglycerate-dependent |
| | phosphoglycerate mutase |
| RSc1956 (murl) | Probable glutamate racemase protein |
| RSc0565 (rfaF) | Probable ADP-heptose—lipopolysaccharide |
| | heptosyltransferase II protein |
| RSc1326 | Probable aspartate aminotransferase protein |
| RSc2464 (clpA) | Probable ATP-dependent protease |
| | (ATP-binding specificity subunit) protein |
| RSc0734 (tolA) | Probable TolA-related transport trans- |
| | membrance protein |
| RSc0736 (pal) | Probable peptidoglycan-associated lipoprotein |
| | precursor |
| RSc0732 (tolQ) | Probable TolQ-related transport trans- |
| | membrane protein |
| RSc2684 (proC) | Probable oxidoreductase pyrroline-5- |
| | carboxylate reductase signal peptide protein |
| RSc1206 | Probable lipoprotein NlpD |
| RSc0500 (ruvB) | Probable holiday junction DNA helicase |
| | RuvB protein |

For the bacteria Xanthomonas oryzae pv. oryzicola, known to infect rice (Oryza sativa) and cause the bacterial leaf streak of rice:

| Essential gene | Description |
|---|---|
| XOCORF_4443 (HrcU) | Type III secretion protein HrcU |
| XOCORF_4444 (HcrV) | Type III secretion protein HrcV |
| XOCORF_4434 (HrcC) | Type III secretion protein HrcC |
| XOCORF_3864 (wxocB) | Putative glycosyltransferase |
| XOCORF_2264 (rpfG) | Two-component system response |
| | regulator RpfG |
| XOCORF_2899 (pilY1) | RpfG |
| XOCORF_3636 (pilQ) | Fimbrial assembly protein |
| XOCORF_3640 (pilM) | Type IV pilus assembly protein |
| XOCORF_1181 (pilZ) | Type IV pilus assembly protein |
| XOCORF_1487 (pilT) | Twitching mobility protein |
| XOCORF_3589 (pgk) | Phosphoglycerate kinase |
| XOCORF_3592 (gapA) | Glyceraldehyde-3-phosphate |
| | dehydrogenase, type I |
| XOCORF_1172 | 3-Oxoacyl-[acyl-carrier-protein] |
| | synthase III |
| XOCORF_2877 | P protein |
| XOCORF_3805 (XpsE) | General secretion pathway protein |
| | E (XpsE) |
| XOCORF_4457 (HrpF) | |
| XOCORF_3672 (ThiE) | Thiamine-phosphate pyrophos- |
| | phorylase (ThiE) |
| XOCORF_3439 (HrpX) | |
| XOCORF_3794 (XpsD) | General secretion pathway protein |
| | D (XpsD) |
| XOCORF_4434 (HrcC-) | HrcC |
| XOCORF_1450 | Conserved hypothetical protein |
| XOCORF_1282 | Dipeptidyl carboxypeptidase I |

For the bacteria Xanthomonas campestris pv. campestris, known to infect all the Brassicaceae (cabbage, broccoli, cauliflower, kale, turnip, oilseed rape, mustard, radish, etc.) and cause the crucifer black rot disease:

| Essential gene | Description |
|---|---|
| B109F06 (pilC) | Fimbrial assembly protein |
| B205G02 (pilB) | Pilus biogenesis protein |
| B202G01 (iroN) | TonB-dependent receptor |
| B302H04 (wxcM) | Bifunctional acetyl transferase/ |
| | isomerase |
| A404F11 (wrcC) | Glycosyltransferase |
| C302F11 (wxcA) | Glycosyltransferase |
| A105E01 (nagA) | N-acetylglucosamine-6- |
| | phosphate deacetylase |
| C110C10 (rmlA) | Glucose-1-phosphate thymidylyl- |
| | transferase |
| A202H06 (xpsM) | General secretion pathway protein M |
| C406A04 (xpsK) | General secretion pathway protein K |
| B502F09 (xpsF) | General secretion pathway protein F |
| B405E02 (xpsE) | General secretion pathway protein E |
| B401G05 (virB8) | VirB8 protein |
| C109G03 (engXCA) | Cellulase |
| B101F11 (rpfG) | Response regulator |
| B203D06 | Transducer protein car |
| A308G04 (trpE) | Anthranilate synthase component I |
| B605H08 (trpD) | Anthranilate synthase component II |
| B504A10 (metA) | Homoserine O-acetyltransferase |
| A506A08 (hisF) | Cyclase |
| C206E04 | Probable restriction modification |
| | system specificity subunit |
| C111B03 (cypC) | Fatty acid alpha hydroxylase |
| A507C08 (fadB) | P-hydroxycinnamoyl CoA hydratase/ |
| | lyase |
| B302H05 (uptB) | Maleylacetoacetate isomerase |
| C307G11 (catB) | Catalase (precursor) |
| B104E02 (icd) | Isocitrate dehydrogenase |
| B106G06 (ppsA) | Phosphoenolpyruvate synthase |
| B401E10 | Conserved hypothetical protein |
| C205C01 | Conserved hypothetical proteins |
| C301C03 | Conserved hypothetical protein |
| B407F08 | Conserved hypothetical proteins |
| B410B02 | Conserved hypothetical proteins |
| B606A8 | Inserted between XC0132 and |
| | XC0133 |
| B310E10 | Inserted between XC0350 and |
| | XC0351 |
| B203A9 | Inserted between XC0408 and |
| | XC0409 (bfeA) |
| A505G9 | Inserted between XC0509 (purD) and |
| | XC0510 (purH) |
| A301D7 | Inserted between XC2898 and |
| | XC2899 (btuB) |
| B306H2 | Inserted between XC3602 and |
| | XC3603 (alkB) |
| A105E4 | Inserted between XC3604 (etf-qo) |
| | and XC3605 |
| C401H6 | Inserted between XC3604 (etf-qo) |
| | and XC3605 |
| B203C8 | Inserted between XC4037 (catB) and |
| | XC4038 (dinG) |

For the bacteria Xanthomonas axonopodis pathovars (e.g. pv. citri and pv. manihotis), known to infect the citrus tree and cassava respectively and cause the citrus canker and the cassava bacterial blight respectively:

| Essential gene | Description |
|---|---|
| XAC1211 (KatE) | monofunctional catalase |
| Xac1815 (XacFhaB) | putative hemagglutinin |
| | transporter protein |
| XAC3600 (wzt) | LPS biosynthesis |
| AvrBs2 | Type three secretion effector |
| XopX | Type three secretion effector |
| XopZ | Type three secretion effector |
| XopAO1 | Type three secretion effector |
| XopN | Type three secretion effector |
| XopQ | Type three secretion effector |

For the bacteria Erwinia amylovora, known to infect the apple and pear tree and cause the fire blight disease:

Essential gene
A7/74SmSp-A20
Ea7/74SmSp-A29
Ea7/74Sm-A33
Ea7174SmSp-A38
Ea7/74Sm-A41
Ea7/74SmSp-A56
Ea7/74SmSp-A64
Ea7/74SmSp-A72
Ea7/74SmSp-A75
Ea7/74SmSp-A76
Ea7/74SmSp-A83

For the bacteria Dickeya dadantii, known to infect potato, tomato, eggplant, chicory and cause the soft rot disease:

| Essential gene | Description |
|---|---|
| Dda3937_00335 (glpD) | glycerol-3-phosphate dehydrogenase |
| Dda3937_03379 (purL) | phosphoribosylformyl-glycineamide |
| | synthetase |
| Dda3937_03564 (opgG) | Glucans biosynthesis protein G precursor |
| Dda3937_00244 (purH) | phosphoribosylaminoimidazole- |
| | carboxamide formyltransferase/IMP |
| | cyclohydrolase |
| Dda3937_00532 (hflK) | FtsH protease regulator |
| Dda3937_02515 (purM) | phosphoribosylaminoimidazole |
| | synthetase |
| Dda3937_02627 | 4-hydroxythreonine-4-phosphate |
| | dehydrogenase |
| Dda3937_00004 (guaB) | IMP dehydrogenase |
| Dda3937_03563 (opgH) | Glucans biosynthesis glucosyl- |
| | transferase H |
| Dda3937_01284 (pyrB) | aspartate carbamoyltransferase |
| Dda3937_03924 (rffG) | dTDP-glucose 4,6-dehydratase |
| Dda3937_01389 (carB) | carbamoyl-phosphate synthase |
| | large subunit |
| Dda3937_03299 (acrA) | MexE family multidrug efflux RND |
| | transporter periplasmic adaptator subunit |
| Dda3937_03300 (acrB) | Multidrug efflux system protein |
| Dda3937_03258 (pyrE) | orotate phosphoribosyltransferase |
| Dda3937_02336 (nlpl) | lipoprotein |
| Dda3937_02506 (nlpB) | outer membrane protein assembly |
| | factor BamC |
| Dda3937_04018 (pta) | phosphate acetyltransferase |
| Dda3937_03554 (pyrC) | dihydro-orotase |
| Dda3937_04573 (lpxM) | acyl (myristate) transferase |
| Dda3937_01116 (glnG) | Nitrogen regulation protein NR(I), |
| | Two-component system |
| Dda3937_02099 (purF) | amidophosphoribosyltransferase |

For the bacteria Xylella fastidiosa, known to infect grapevine and olive trees and cause the pierce's disease on grapevine, the citrus variegated chlorosis, or the olive quick decline syndrome:

| Essential gene | Description |
|---|---|
| rpfC | two-component regulatory protein |
| (PD1709) MopB | major outer membrane protein |
| LesA | putative lipase/esterase |
| PD1703 (LipA) | lipase |
| PD0928 (Zot) | Zonula occludens toxin |
| PD0986 | hemagglutinin-like protein |
| gumD | Exopolysaccharides biosynthesis gene |
| gumH | Exopolysaccharides biosynthesis gene |
| xhpT | response regulators |
| PD0528 (XatA) | autotransporter protein |
| PD0279 (cgsA) | cyclic di-GMP synthase A |
| PD0062 (FimA) | Fimbrial adhesions protein |
| PD0058 (FimF) | Fimbrial adhesions protein |
| PD0731 (XadA) | encoding Xanthomonas adhesin- |
| | like protein A |
| PD1792 (HxfB) | hemagglutins |

For the bacteria Candidatus liberibacter solanacearum, known to infect potato and to cause Zebra Chip disease, and Candidatus liberibacter asiaticus (or americanus/africanus), known to infect citrus and to cause Citrus greening disease:

Candidatus Liberibacter solanacearurn

| Essential gene* | Description |
|---|---|
| WP_013462130 | VWA domain-containing protein |
| WP_076969829 | unknown protein |
| WP_013461676 | endonuclease/exonuclease/ |
| | phosphatase family protein |
| WP_076969883 | unknown protein |
| WP_013462162 | OmpA family protein |
| WP_080550991 | TadE/TadG family protein |
| WP_076969829 | unknown protein |
| WP_076970537 | unknown protein |
| WP_034443047 | unknown protein |
| WP_034441878 | unknown protein |
| WP_076970544 | VWA domain-containing protein |
| WP_075969422 | unknown protein |
| WP_103846864 | VWA domain-containing protein |
| ONI59292 | serralysin |
| WP_013462515 | unknown protein |
| WP_045960760 | unknown protein |
| WP_013462289 | unknown protein |
| WP_076969829 | unknown protein |
| WP_013461834 | Na+/H+ antiporter NhaA |
| WP_013461833 | ferredoxin-NADP+ reductase protein |

*Genes denoted as WP_MCXXXXXXXXX correspond to “protein_id” of the gene in Candidatus Liberibacter solanacearum CLso-ZC1, complete genome with Genbank accession number NC_014774.1 of Apr. 17, 2017

Candidatus Liberibacter asiaticus

| | |
|---|---|
| WP_012778355 | unknown protein |
| WP_012778427 | unknown protein |
| WP_012778510 | unknown protein |
| WP_012778517 | unknown protein |
| ACT56857 | Serralysin |
| WP_012778607 | VWA domain-containing protein |
| WP_012778668 | unknown protein |
| WP_015452668 | VWA domain-containing protein |
| WP_015452678 | unknown protein |
| WP_015452727 | unknown protein |
| WP_015824938 | unknown protein |
| WP_015824940 | unknown protein |
| WP_015452765 | VWA domain-containing protein |
| WP_015452784 | OmpA family protein |
| WP_015452797 | unknown protein |
| WP_015452837 | unknown protein |
| WP_015824957 | unknown protein |
| WP_015452939 | VWA domain-containing protein |
| ACT56835 | Na+/H+ antiporter NhaA |
| ACT56837 | ferredoxin-NADP+ reductase protein |

* Genes denoted as WP_XXXXXXXXX correspond to “protein_id” of the gene in Candidatus Liberibacter asiaticus str. psy62, complete genome with Genbank accession number NC_012985.3 of Mar. 30, 2017

Any of these genes can be the target of the iRNAs of the invention.

In beneficial bacteria, the bacterial genes to be targeted are for examples: genes required for phage production that negatively regulate bacterial survival (e.g. phage baseplate assembly protein GpV), NolA and NodD2 genes from Bradyrhizobium japonicum that are known to reduce the expression of nod genes at high population densities and therefore to decrease Nod production, a bacterial signal that is essential for symbiotic invasion (knocking-down these genes from inoculant strains should thus result in competitive nodulation), the small non-coding RNA spot42 encoded by the spot forty-two (spj) gene that controls carbohydrate metabolism and uptake (knocking-down this gene from a given bacterium should result in an increased bacterial titer).

In vascular bacteria, the bacterial genes to be targeted are for example type III secretion genes.

When the targeted bacteria are phytopathogenic bacteria, said essential or virulence bacterial genes can be structural genes of secretion systems including the type III secretion system (e.g. HrcC),
genes that are required for the proper function of the type VI secretion system (e.g. TssB),
genes encoding master regulators of bacterial effector expression (e.g. HrpL, HrpG, HrpX),
genes encoding factors required for the biosynthesis of phytotoxins (e.g. Cfa6, which is important for coronatine biosynthesis in some Pseudomonas syringae pathovars),
genes required for the biosynthesis of virulence compounds (e.g. XpsR, which is important for the biosynthesis of exopolysaccharide EPS1, two-component system RavS/RavR).

The iRNAs of the invention share advantageously sequence homologies with any of these essential genes or virulence genes from the targeted bacterial pathogen species.

As used herein, the term “sequence homology” refers to sequences that have sequence similarity, i.e., a sufficient degree of identity or correspondence between nucleic acid sequences. In the context of the invention, two nucleotide sequences share “sequence homology” when at least about 80%, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, alternatively at least about 99% of the nucleotides are similar.

Conversely, nucleotide sequences that have “no sequence homology” are nucleotide sequences that have a degree of identity of less than about 10%, alternatively of less than about 5%, alternatively of less than 2%.

Preferably the similar or homologous nucleotide sequences are identified by using the algorithm of Needleman and Wunsch. Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide residue matches and an identical percent sequence identity when compared to the corresponding alignment generated by GAP Version 10.

Of note, the iRNAs of the invention do not inhibit genes that are expressed in eukaryotic cells, or in fungi, insects, pests or other plant-infecting pathogens. Specifically, the iRNAs of the invention do not inhibit the expression of oncogenes that have bacterial origin and are inserted into other genomes. More precisely, the iRNAs of the invention do not inhibit the expression of the oncogenes iiaM and ipt of the Agrobacterium tumefaciens bacteria.

### Chimeric Silencing Elements

For protecting plants against diseases caused by several bacterial pathogens, the method of the invention advantageously uses functional iRNAs carrying sequence homologies with more than one bacterial gene (hereafter referred to as “chimeric iRNAs”). These chimeric iRNAs preferably share homology with at least two, three, four, or more bacterial essential genes and/or virulence factors, such as those described above.

Examples of such chimeric iRNAs include:

(1) A chimeric dsRNA having at least 80% sequence homology with the HrpB, HrcC, XpsR and TssB genes from R. solanacearum encoding factors involved in transcriptional activation of bacterial effectors (i.e. HrpB), in the assembly of the TTSS (i.e. HrcC), in the biosynthesis of exopolysaccharide EPS1 (i.e. XpsR), and in the proper function of the type VI secretion system (i.e. TssB). In particular a dsRNA having the sequences SEQ ID NO: 20 (sense strand) and 21 (antisense strand), preferably a hairpin RNA in which the two strands are connected by a loop corresponding to SEQ ID NO: 2;
(2) A chimeric dsRNA having at least 80% sequence homology with the HrpG, HrpX and RsmA genes from Xanthomonas campestris or Xanthomonas oryzae, in particular from Xanthomonas campestris pv. campestris or Xanthomonas oryzae pv. oryzae: HrpG encodes a regulator of HrpX, which is required for the transcriptional activation of Xanthomonas effectors and RsmA encodes an RNA-binding protein that plays a critical role in Xanthomonas pathogenicity and in the proper expression of virulence factors. In particular a dsRNA having to the sequences SEQ ID NO: 22 (sense strand) and 23 (antisense strand), preferably a hairpin RNA in which the two strands are connected by a loop corresponding to SEQ ID NO: 2;
(3) A chimeric dsRNA having at least 80% sequence homology with the essential genes RpoB, RpoC and FusA from different Pseudomonas syringae pathovars including Pto DC3000 and Pseudomonas syringae pv. CC440. In particular a dsRNA having to the sequences SEQ ID NO: 24 (sense strand) and 25 (antisense strand), preferably a hairpin RNA in which the two strands are connected by a loop corresponding to SEQ ID NO: 2;
(4) A chimeric dsRNA having at least 80% sequence homology with the essential genes SecE, RpoA and RplQ from different Pseudomonas syringae pathovars including Pto DC3000 and Pseudomonas syringae pv. CC440. In particular a dsRNA having to the sequences SEQ ID NO: 26 (sense strand) and 27 (antisense strand), preferably a hairpin RNA in which the two strands are connected by a loop corresponding to SEQ ID NO: 2;
(5) A chimeric dsRNA having at least 80% sequence homology with the essential genes NadHb, NadHd and NadHe from different Xanthomonas pathovars. In particular a dsRNA having to the sequences SEQ ID NO: 28 (sense strand) and 29 (antisense strand), preferably a hairpin RNA in which the two strands are connected by a loop corresponding to SEQ ID NO: 2;
(6) A chimeric dsRNA having at least 80% sequence homology with the essential genes DnaA, DnaE1 and DnaE2 from different Xanthomonas pathovars. In particular a dsRNA having to the sequences SEQ ID NO: 30 (sense strand) and 31 (antisense strand), preferably a hairpin RNA in which the two strands are connected by a loop corresponding to SEQ ID NO: 2;

Other examples of chimeric iRNAs targeting these genes include:
a dsRNA targeting concomitantly the HrpG, HrpB and HrcC genes of Ralstonia solanacearum, in particular a dsRNA having to the sequences SEQ ID NO: 18 (sense strand) and 19 (antisense strand), preferably a hairpin RNA in which the two strands are connected by a loop corresponding to SEQ ID NO: 2;
a dsRNA targeting concomitantly the HrpL and Cfa6 genes of Pto DC3000, in particular a dsRNA having the sequences SEQ ID NO: 1 (sense strand) and 3 (antisense strand), preferably a hairpin RNA in which the two strands are connected by a loop corresponding to SEQ ID NO: 2;
a dsRNA targeting concomitantly the AvrPto and AvrPtoB genes of Pto DC3000, in particular a dsRNA having the sequences SEQ ID NO: 14 (sense strand) and 15 (antisense strand), preferably a hairpin RNA in which the two strands are connected by a loop corresponding to SEQ ID NO: 2;
a dsRNA targeting concomitantly the LuxA and LuxB genes of lux-tagged Pto DC3000, in particular a dsRNA having the sequences SEQ ID NO: 108 (sense strand) and 109 (antisense strand), preferably a hairpin RNA in which the two strands are connected by a loop corresponding to SEQ ID NO: 2;

In a preferred embodiment, the iRNA of the invention is a chimeric iRNA inhibiting at least one gene encoding a virulence factor or an essential gene of bacterial cells as defined above, together with at least one other gene encoding a virulence factor or an essential gene of other pathogens or parasites known to be sensitive to HIGS. It can be also a gene required for the biosynthesis of toxic secondary metabolites from non-bacterial pathogens or plant parasites.

Said other gene is for example chosen in the group consisting of: known fungicide targets (e.g. Cytochrome P450 lanosterol C-14α-demethylase (CYP51) from Fusarium graminearum, the causal agent of Fusarium head blight (FHB) (20); essential genes or pathogenicity factors from unrelated fungi (e.g. DCL1 and DCL2 from the necrotrophic fungi Botrytis cinerea and Verticilium dahliae (15)), Chitin synthase Ch3b from Fusarium graminearum (35), β-1,3-Glucan synthase (FcGls1), Mitogen-activated protein kinase 1 (FsFmk1), Mysosin motor domain-containing chitin synthase V (FsChsV) from Fusarium culmorum (36), MAP kinase (PtMAPK1) or Cyclophilin1 (PtCYC1) from Puccinia triticina (37), the causal agent of wheat leaf rust and PsCPK1 from Puccinia striiformis (38), causing wheat stripe rust); essential genes or pathogenicity factors from oomycete pathogens (e.g. Highly Abundant Message 34 (HAM34) or Cellulose Synthase (CES1) from Bremia lactucae, causing downy mildew of lettuce (39)); genes required for the biosynthesis of a key toxic secondary metabolite such as the AOC gene, which is required for aflatoxin biosynthesis in Aspergillus species and that is sensitive to HIGS technology in transgenic maize (40).

In another preferred embodiment, the method of the invention uses: (i) one or more iRNAs targeting a widespread sequence region of an essential or virulence gene that is conserved in a large set of bacterial pathogens or (ii) one or more iRNAs targeting genes that are essential or virulence factors from unrelated bacterial pathogens. Such particular embodiment of the method confers broad-spectrum protection towards multiple bacterial pathogens. The iRNAs of the invention are advantageously long dsRNAs, miRNAs and/or siRNA as defined above.

In a particular embodiment, the method of the invention further comprises introducing into the plants one or more dsRNAs targeting one or multiple genes of parasite(s) that are different from bacteria, such as viruses, fungi, oomycetes, insects or nematodes. In this embodiment, the iRNAs are directed to an essential gene or to a virulence gene of the parasite(s). The one or more iRNAs targeting the genes of the parasite(s) is/are advantageously delivered concomitantly or co-expressed with the iRNA targeting the bacterial gene(s). Such a method is useful for concomitant prevention or treatment of diseases caused by bacterial pathogens and other parasites. This method can be carried out using chimeric iRNAs carrying sequence homologies with bacterial but also other pathogenic/parasitic genes, as proposed above, or a cocktail of iRNA molecules, some bearing homologies to bacterial genes and other bearing homologies to genes from other pathogens/parasites.

### Vectors of the Invention

In one preferred embodiment, the long and small RNAs of the invention are isolated, naked RNA molecules that are used directly on producer plant cells and on target bacterial cells respectively.

In another preferred embodiment, the long RNAs of the invention are encoded by recombinant DNA constructs that facilitate the introduction into a plant cell and/or facilitate the expression of long RNAs in said plant cell. Said recombinant constructs can be a plasmid or a vector, which may be commercially available. It is preferably a plant expression vector as described below.

The present invention thus also relates to a plant recombinant DNA vector (or “plant DNA construct”) or a plant viral vector comprising a polynucleotide sequence encoding at least one functional interfering RNA (iRNA) inhibiting the expression of at least one bacterial gene, wherein said polynucleotide sequence is expressible in eukaryotic cells.

Said functional iRNA is as defined above, either a short or long dsRNA, a long ssRNA, a siRNA or miRNA, preferably said functional iRNA is a long dsRNA, a long ssRNA, a siRNA or a miRNA.

Said at least one bacterial gene is preferably an essential or a virulence bacterial gene as defined above.

In an embodiment, the vector is a DNA vector. Said DNA vector comprises advantageously a transcription unit comprising: a transcription initiation region, a transcription termination region, and the polynucleotide encoding the iRNA of the invention, wherein said polynucleotide sequence is operably linked to said initiation and termination regions in a manner that allows the expression of the iRNA molecule in the eukaryotic cell.

In a preferred embodiment, said eukaryotic cell is a plant cell that is able to express high amounts of iRNAs, such as N. benthamiana leaves that are well adapted for Agrobacterium-mediated transient transformation.

The DNA vector of the invention may encode one or both strands of the iRNA molecule of the invention, or a single self-complementary strand that self-hybridizes into a dsRNA duplex. The transcription initiation region may be from a promoter for a eukaryotic RNA polymerase II or III (pol II or III) including viral promoters active in plant cells such as the CaMV 35S promoter, since transcripts from these promoters are expressed at high levels in all cells of the plant organisms. A large choice of promoters suitable for expression of heterologous genes in plant cells are available in the art. They can be obtained for instance from plant viruses. They include constitutive promoters, i.e. promoters which are active in most tissues and cells and under most environmental conditions, as well as tissue-specific or cell-specific promoters which are active only or mainly in certain tissues or certain cell types, and inducible promoters that are activated in response to chemical stimuli. Organ or tissue specific promoters that can additionally be used in the present invention for plant protection against bacterial pathogens include in particular promoters that are active in tissues/cell types that are relevant for the entry and the propagation of bacterial pathogens, for example in hydathodes, guard cells, xylem parenchyma cells and cells surrounding the base of trichomes.

Said transcription termination region is preferably recognized by a eukaryotic RNA polymerase, more preferably by Pol II or Pol III. For example, said transcription termination can be a TTTTT sequence.

Large numbers of DNA vectors suitable for dsRNA molecule expression are known to those of skill in the art and commercially available. The selection of suitable vectors and the methods for inserting DNA constructs therein are well known. The recombinant vectors capable of stably expressing the dsRNA molecules can be transformed in planta, and persist in target cells. The choice of the vector depends on the intended host and on the intended method of transformation of said host.

In an embodiment, the vector is a viral vector. Said viral vector is preferably selected from various recombinant plant RNA viruses (e.g. Tobacco mosaic virus, Tobacco rattle virus, Potato virus X, Barley stripe mosaic virus, Tomato bushy shunt virus), which can be used to produce high amount of small RNAs by plant cells through VIGS (11). Here also, the choice of the viral vector depends on the intended host and on the intended method of infection of said host. These recombinant plant virus trigger the in planta production of small RNAs that can inhibit the expression of at least one bacterial gene target.

The present invention also encompasses recombinant DNA vectors or viral vectors including one or more marker genes, which allows selecting the transformed host cells.

In a preferred embodiment, the DNA or viral vector of the invention comprises a polynucleotide sequence encoding two, three, or four functional interfering RNA (iRNA) genes as defined above, therefore being able to inhibit two, three, or four different bacterial genes. The skilled person can identify the best combinations of iRNA by conventional means. Combinations of more than four targeted genes are also encompassed within the present invention.

In one embodiment, the DNA vector of the invention comprises at least one of the sequences SEQ ID NO: 1-31 and 108-109, in particular at least one of the sequences SEQ ID NO: 1-31, and preferably the sequence systems: SEQ ID NO: 1-2-3; 14-2-15; 18-2-19; 20-2-21; 22-2-23; 24-2-25; 26-2-27; 28-2-29; 30-2-31, 108-2-109, more preferably the sequence systems: SEQ ID NO: 1-2-3; 14-2-15; 18-2-19; 20-2-21; 22-2-23; 24-2-25; 26-2-27; 28-2-29; 30-2-31.

In another embodiment, the DNA vector of the invention comprises at least one of the sequences SEQ ID NO: 88-97, preferably the sequence systems: SEQ ID NO: 88-89, 90-91, 92-93, 94-95, 96-97.

The DNA vector of the invention can be prepared by conventional methods known in the art. For example, it can be produced by amplification of a nucleic sequence by PCR or RT-PCR, by screening genomic DNA libraries by hybridization with a homologous probe, or else by total or partial chemical synthesis. The recombinant vectors can be introduced into host cells by conventional techniques, which are known in the art.

### Antibacterial Methods and Uses of the Invention

In another aspect, the present invention relates to an in vitro method for inhibiting the expression of at least one gene in a target bacterial cell, said method comprising the step of contacting said target bacterial cell with one or more of the small RNAs of the invention or with compositions comprising same. In case of virulence factors that are transcriptionally activated in the contact of plant cells, specific medium will be used (e.g. minimal media known to activate the expression of phytotoxins and transcription factors required for the Hrp gene expression, or presence of leaf pieces as in the semi-in vivo stomatal reopening assays of the invention).

In other words, the present invention relates to the in vitro use of small RNAs or of a composition comprising small RNAs, for inhibiting the expression of at least one gene in a target bacterial cell, wherein said target bacterial cell is contacted directly with said small RNA or with said composition.

Preferably, said small RNA is a single-stranded or a double-stranded siRNA or a single-stranded or a double-stranded miRNA duplex. More preferably, said small RNA inhibits the expression of at least one gene encoding a virulence factor or an essential gene or an antibacterial resistance gene if said bacterial cell is pathogenic, or inhibits the expression of at least one gene encoding a repressor of growth or a negative regulator of a pathway that is useful for the host if said bacterial cell is beneficial.

Preferably, said composition contains plant extracts obtained from producer plant cells that express at least one long dsRNA that is specific to at least one gene of said bacterial cell. More preferably, said composition contains total RNAs, or total small RNAs, or apoplastic fluids, or extracellular vesicles, or extracellular free small RNAs recovered from said plant cells. Said producer plant cells are for example chosen in the group consisting of Tobacco (e.g. Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum), Cottonseed, Cotton, Bean, Banana/plantain, Sorghum, Pea, Sweet potatoes, Soybeans, Cabbage, Cassava, Onion, Melon, Oats, Peanut, Sunflower, Palm oil, Rye, Citrus, Wheat, Peppers, Yams, Olives, Grapes, Sesame, Sugarcane, Sugarbeet, Pea and Coffee, Orange trees, Apple trees, Citrus trees, Olive trees etc.

Ornamental plants can also be used for producing the said small RNAs of the invention. These ornamental plants are e.g., Chrysanthemum (such as Chrysanthemum morifolium), Impatiens, Geranium, Pelargonium, Phlox, Rhododendron anthurium spp, Rose tree, Curcumas, Anthuriums, Begonia, Hibiscus rosa-sinensis, Amaryllis, Calla, Cyclamen, Dracaena, etc.

By “inhibiting the expression of at least one gene”, it is herein meant that the expression of said gene is reduced, i.e., the mRNA or protein levels of the target sequence is statistically lower than the mRNA level or protein level of the same target sequence in appropriate control bacteria which is exposed to control small RNAs targeting unrelated genes (e.g. fungal genes). In particular, reducing the mRNA polynucleotide level and/or the polypeptide level of the target gene in a bacteria according to the invention results in reaching less than 60%, less than 50%, less than 40%, less than 30%, less than 20%, less than 10%, or less than 5% of the mRNA polynucleotide level, or the level of the polypeptide encoded thereby, of the same target sequence in an appropriate control bacterium. Methods to assess the expression level of the RNA transcript, the expression level of the polypeptide encoded by the targeted gene, or the activity of said polynucleotide or polypeptide are well-known in the art.

In one embodiment, the small RNAs of the invention comprise at least one of the sequences SEQ ID NO: 1-31, and 108-109, in particular at least one of the sequences SEQ ID NO: 1-31, and preferably the sequence systems: SEQ ID NO: 1-2-3; 14-2-15; 18-2-19; 20-2-21; 22-2-23; 24-2-25; 26-2-27; 28-2-29; 30-2-31, 108-2-109, more preferably the sequence systems: SEQ ID NO: 1-2-3; 14-2-15; 18-2-19; 20-2-21; 22-2-23; 24-2-25; 26-2-27; 28-2-29; 30-2-31.

In another embodiment, the small RNAs of the invention comprise at least one of the sequences SEQ ID NO: 88-97, preferably the sequence systems: SEQ ID NO: 88-89, 90-91, 92-93, 94-95, 96-97.

In this aspect, any type of bacteria can be targeted. Pathogenic or beneficial bacteria can be targeted, as described above.

In one embodiment, this method is of particular interest for inhibiting or limiting the pathogenicity and growth of pathogenic bacteria in a sample. It is also useful for killing pathogenic bacterial cells in a sample.

In another embodiment, this method can also be used for promoting the replication of beneficial bacteria by inhibiting genes that negatively regulate directly or indirectly bacterial growth, as mentioned above.

In another embodiment, it is also possible to use this method for restoring the sensitivity of bacterial cells to an antibacterial compound by targeting a gene that is involved in the bacterial resistance to said antibacterial compound.

By “antibacterial compound”, it is meant a compound that is used or proposed for killing bacteria. Classical antibacterial compounds that are used in the phytosanitary field are for example copper-based bactericides or secondary metabolites derived from macro- and micro-organisms.

The amount of small RNAs to be used typically depends on the number of bacteria and on the type of bacteria that are targeted. This amount can be comprised between 10 and 30 ng/μl of total RNAs containing the effective small RNAs.

### Phytotherapeutic Methods and Uses of the Invention

According to the invention, the one or more iRNAs of the invention is/are introduced into plant cells by using the standard methods mentioned above for expressing nucleic acids. A variety of methods for genetic transformation of eukaryotic cells are available in the art for many plant species. By way of non-limitative examples, one can mention projectile bombardment, virus-mediated transformation, Agrobacterium-mediated transformation, and the like. Electroporation is not included.

The term “introduced” in the context of inserting a nucleic acid into a cell, means “transfection” or “transformation” or “transduction” and includes reference to the incorporation of a nucleic acid into a eukaryotic cell where the nucleic acid may be stably incorporated into the genome of the cell (e.g., chromosome, plasmid), or transiently expressed (e.g., transient delivery of a gene construct via Agrobacterium tumefaciens).

The expression of the iRNAs of the invention in the host plant cell may be transient or stable. Stable expression refers in particular to the preparation of transgenic plants using conventional techniques.

Said iRNA will be processed into siRNA or miRNA duplexes by using the plant Dicer-like enzymes and other small RNA processing factors. Said small RNAs duplexes and/or mature small RNA guides (i.e. loaded into AGOs) are thereafter translocated in the extracellular medium, or at the surface of the plant cells, where they might encounter the bacterial cells.

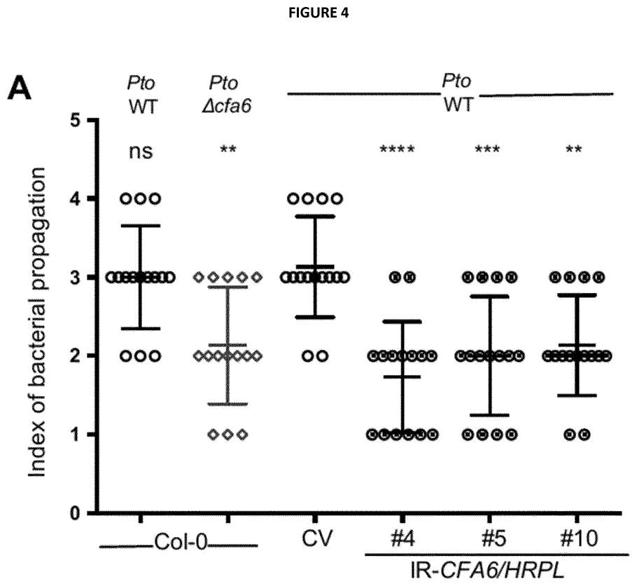

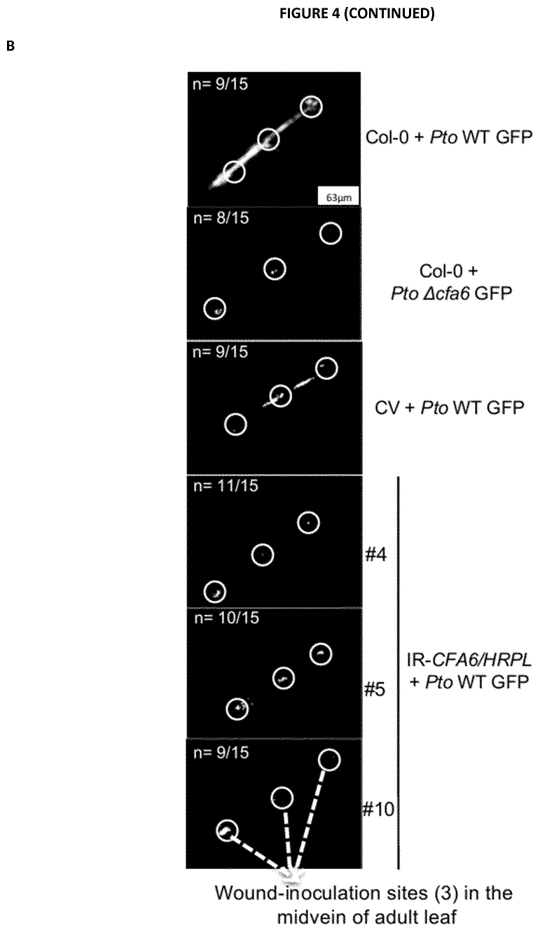

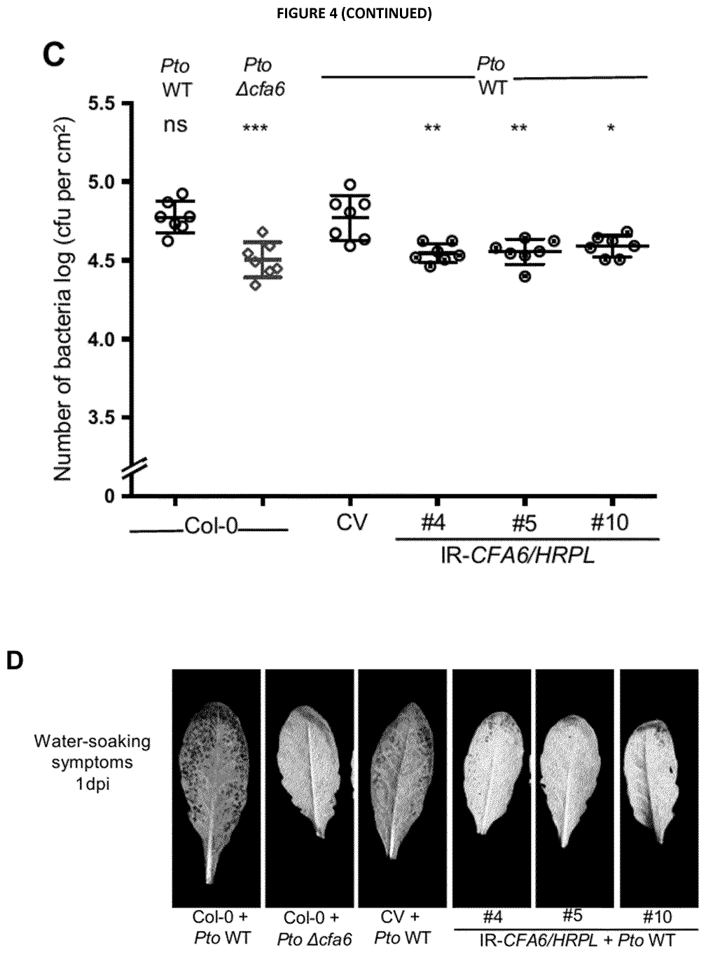

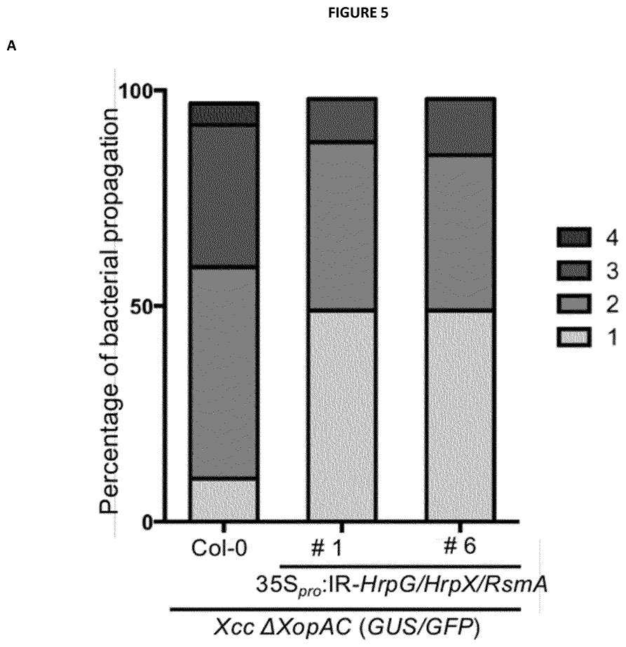

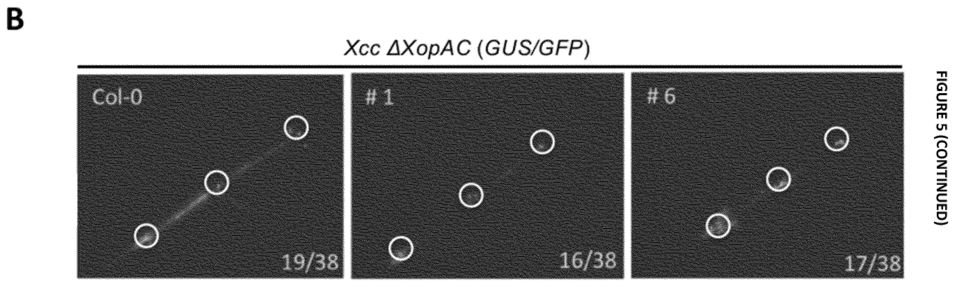

As demonstrated in the examples below (examples 4, 5 and FIGS. 4-6), the growth and the virulence of bacterial cells are both decreased when placed in contact with the plant cells of the invention in conditions where the mature iRNAs of the invention are secreted.

In one aspect, the present invention relates to a method for treating target plants against a bacterial infection, said method comprising the step of introducing into at least one cell of said target plant a long dsRNA molecule targeting specifically a virulence bacterial gene or an essential bacterial gene or an antibacterial resistance gene.

In another aspect, the present invention relates to an RNAi-based biocontrol method for treating plants against bacterial infection, said method comprising the step of delivering small RNAs, or a plant extract containing such small RNAs or a composition comprising these small RNAs (e.g. total RNAs extracted from plant cells or tissue stably or transiently expressing these small RNA entities) on plant tissues prior to and/or after bacterial infection.

Preferably, said composition contains plant extracts obtained from plant cells that express at least one long dsRNA that is specific to said at least one virulence or essential or antibacterial resistance bacterial gene. More preferably, said composition contains EVs recovered from said plant extracts, or extracellular free small RNAs secreted by said plant cells and recovered in said plant extracts, or apoplastic fluids recovered from said plant extracts. Even more preferably, said composition is a liquid sprayable composition.

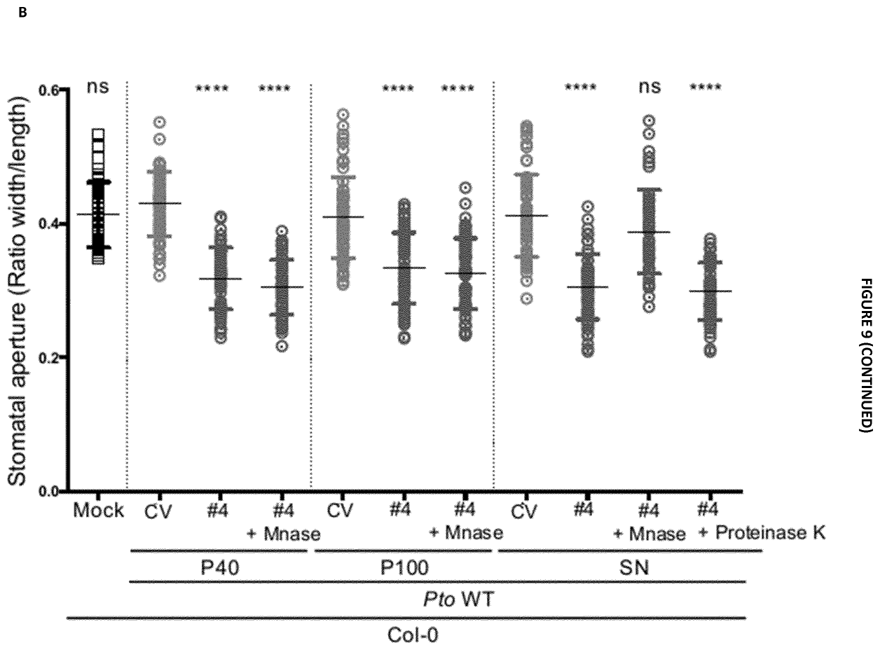

In this aspect, the bacterial cells are eventually contacted directly with small RNAs (i.e., siRNAs or miRNAs) that will be able to cross the bacterial double-membrane in the case of Gram negative bacteria and reach the cytosol of bacterial cells where the targeted gene(s) will be silenced in a sequence-specific manner, thereby resulting in the dampening of bacterial pathogenicity (see examples 5-7 & FIGS. 4-6 & FIGS. 9-10).

As used herein, the term “small RNAs” designates the small RNAs carrying the inhibiting activity of the iRNAs of the invention. Specifically, they are siRNAs or miRNAs (duplexes or simplexes) that share at least 80% sequence homology with at least one bacterial gene, preferably with at least one bacterial virulence or an essential gene, more preferably with at least one of the genes cited above. These small RNAs generally comprise no more than 40 base pairs. Preferably, they contain between 18 and 30, more preferably between 18 and 25 base pairs. More preferably, said small RNAs specifically inhibit at least one of the bacterial essential or virulence gene defined above.

Preferably, these small RNAs are double-stranded siRNAs, as disclosed above. These double-stranded RNAs can be embedded in extracellular vesicles and/or associated with RNA binding proteins.

Another aspect of the invention relates to the use of at least one iRNA or a vector containing this iRNA, as defined above, as a phytotherapeutic agent. Preferably, said iRNA or vector is used for treating a disease caused by a pathogenic bacterium in plants or for preventing a bacterial infection in plants.

In one embodiment, this phytotherapeutic iRNA is a short or long dsRNA, a siRNA duplex or miRNA duplex, a siRNA simplex or a miRNA simplex, as defined above. In yet another embodiment, the iRNA targets bacterial genes and genes of other non-bacterial pathogens or parasites, as defined above, for concomitant prevention or treatment of diseases caused by bacterial pathogens and other pathogens/parasites in plants. All the embodiments proposed above for the iRNAs, the vectors, and the transformation methods are herewith encompassed and do not need to be repeated.

In the context of phytosanitary technical problem, said small RNAs can be delivered to the plant tissues by various means (e.g., by spray). They can be embedded within microspheres, nanoparticules, liposomes or natural exosomes. Preferred formulations are disclosed below.

### Transgenic Plants of the Invention

The plant cells transformed with the iRNAs of the invention and able to generate the small RNAs of the invention are hereafter designated as “plant cells of the invention” or “host cells of the invention”. They contain at least one iRNA (preferably a long RNA) containing at least one sequence targeting specifically a bacterial gene, e.g., a virulence or essential bacterial gene, or a DNA construct or vector as defined above. They are able to generate and secrete small RNAs inhibiting bacterial virulence, pathogenicity and proliferation.

Plants that have been stably transformed with a transgene encoding the long RNAs may be supplied as seed, reproductive material, propagation material, or cell culture material which does not actively express the long RNA but has the capability to do so.

If they are only used for producing the small RNAs of the invention, they can be called “producer plant cells”. If they will beneficiate from the antibacterial effect conferred by the produced small RNAs, they can also be called “target plants”. Both type of plants (the producer and the target ones) are recombinant cells expressing and producing the small RNAs of the invention.

The term “plant” herein encompasses a plant cell, a plant tissue, a plant part, a whole plant, ancestors and progenies thereof. A plant part may be any part or organ of the plant and includes for example seed, fruit, stem, leaf, shoot, flower, anther, root, tuber and petiole. The term “plant” also encompasses suspension cultures, embryos, meristematic, regions, callus tissue, gametophytes, sporophytes, pollen and microspores. It refers to all plants including ferns and trees.

In another aspect, the present invention relates to an isolated plant cell or to a transgenic plant stably or transiently expressing at least one functional iRNA of the invention. It also relates to an isolated plant cell containing a DNA or viral vector of the invention. Said plant cell may be a genetically modified cell obtained by transformation with said DNA vector. Examples of transformation processes are Agrobacterium-mediated transformation or shot-gun-mediated transformation.

Said plant cell may also be a modified cell obtained by infection or, alternatively, by Agrobacterium-mediated transformation with said viral vector.

The DNA encoding the iRNA may be transiently expressed or stably integrated in the plant cell chromosome.

All the embodiments proposed above for the plant cells, the iRNAs, the vectors, and the transformation methods are herewith encompassed and do not need to be repeated.

Methods to generate such transgenic plants are disclosed in the example part below. They contain the step of:

i) transforming a plant cell with a DNA vector expressing at least one functional interfering RNA of the invention, or
ii) infecting a plant cell with a plant virus, preferably an plant RNA virus, expressing at least one functional interfering RNA of the invention,
for a sufficient time (typically 3-4 days for a tobacco plant) for the plant cell to stably or transiently express a significant amount of small RNAs.

By “significant amount”, it is herein meant an amount that has been shown to have an antibacterial effect in a test such as described above. This significant amount is preferably comprised between 10 and 30 ng/μ1 of total RNAs expressing the effective small RNAs.

In particular, said transgenic plant is capable of host-induced gene silencing of a bacteria, and contains an expressible mature small RNA capable of down-regulating or suppressing the expression of at least one gene of a bacteria. As demonstrated by the inventors, said small RNAs are capable of propagating across or crossing the double membrane of the targeted bacteria.

In another aspect, the present invention relates to a target transgenic plant stably or transiently expressing the mature small RNAs of the invention. In one embodiment, said target transgenic plant contains the DNA vector of the invention. In one preferred embodiment, said target plant is Rice, Maize, Barley, Cottonseed, Cotton, Bean, Banana/plantain, Sorghum, Pea, Sweet potatoes, Soybeans, Cabbage, Cassava, Potato, Tomato, Onion, Melon, Oats, Peanut, Sunflower, Palm oil, Rye, Citrus, Wheat, Peppers, Yams, Olives, Grapes, Taro, Tobacco, Sesame, Sugarcane, Sugarbeet, Pea and Coffee, Orange trees, Apple trees, Citrus trees, and Olive trees. All the embodiments proposed above for the iRNAs, the vectors, and the transformation techniques are herewith encompassed and do not need to be repeated.

Ornamental plants can also be used as a target for the small RNAs of the invention. These ornamental plants are e.g., Chrysanthemum (such as Chrysanthemum morifolium), Impatiens, Geranium, Pelargonium, Phlox, Rhododendron anthurium spp, Rose tree, Curcumas, Anthuriums, Begonia, Hibiscus rosa-sinensis, Amaryllis, Calla, Cyclamen, Dracaena, etc.

On another hand, “producer plants” stably or transiently expressing the mature small RNAs of the invention are chosen among: Tobacco (e.g. Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum). Preferred producer plants are Tobacco, Taro and Giger.

### Compositions Comprising the Silencing Elements of the Invention

In another aspect, the present invention relates to phytotherapeutic compositions containing the small RNAs of the invention and optionally a physiological or agronomical acceptable carrier, excipient or diluent. In particular, it relates to phytotherapeutic compositions containing a significant amount of siRNAs or miRNAs inhibiting the expression of at least one bacterial gene, preferably inhibiting one essential or virulence bacterial gene or antibacterial resistance gene and optionally a physiological or agronomical acceptable carrier, excipient or diluent and/or a bactericidal compound.

The small RNAs contained in the phytotherapeutic compositions of the invention may be synthetic or may be obtained from plants, plant tissues or plant cells stably or transiently expressing said small RNAs. In particular, plants, plant tissues or plant cells stably or transiently transformed by a DNA vector of the invention or infected by a viral vector of the invention will produce small RNAs. A phytotherapeutic composition of the invention may thus comprise either total RNAs of plants, plant tissues or plant cells stably or transiently expressing the small RNAs of interest, or a purified small RNA fraction of the total RNAs or synthetic RNAs produced by conventional means.

For producing total RNAs of plants, plant tissues or plant cells stably or transiently expressing small RNAs of the invention, a DNA or viral vector of the invention may be used to transform said plants, plant tissues or plant cells, as described above under the section “Transgenic plants”. Total RNAs are then extracted by methods known in the art.

In one particular aspect, the present invention thus also relates to a composition comprising a DNA or viral vector of the invention, as defined above, which may be used for producing the small RNAs of the invention in plants, and thus to produce eventually the phytotherapeutic compositions of the invention.

These compositions are preferably used for manufacturing phytotherapeutic compositions in methods comprising the steps of:

a) generating, as disclosed above, recombinant transgenic plant cells producing siRNAs or miRNAs inhibiting specifically a bacterial essential gene or a bacterial virulence gene or an antibiotic resistance gene of a phytopathogenic bacterium,
b) recovering the cell plant extract or the extracellular vesicles or the apoplastic fluids or the extracellular free small RNAs from said recombinant plant cells, containing a significant amount of said siRNAs or miRNAs,
c) optionally, adding an excipient or another active principle in said phytotherapeutic composition.

By “significant amount”, it is herein meant an amount that has been shown to have an antibacterial effect in a test such as described above. This amount is preferably comprised between 10 and 30 ng/μ1 of total RNAs expressing the effective small RNAs.

As explained above, it is possible to use phytotherapeutic compositions containing dsRNA precursors (short or long, depending on the resulting small RNA species) or other precursors of small RNAs that will be potentially processed and perhaps amplified through the action of plant RNA-dependent RNA polymerases coupled with DCL-directed processing of dsRNAs leading to effective antibacterial small RNAs.

The present invention therefore also relates to a phytotherapeutic composition containing a significant amount of small RNAs inhibiting the expression of an essential bacterial gene, or of a virulence bacterial gene or of an antibacterial resistance bacterial gene.

These small RNAs are preferably contained within total RNA extracts, or extracellular vesicles, or apoplastic fluids or extracellular free small RNAs fractions from the transgenic plant of the invention.

The present invention relates to the use of these compositions for inhibiting or preventing the growth or pathogenicity of bacteria on target plants.

In one embodiment, the silencing element of the invention can be provided as an external composition such as a spray to the plant, plant part, seed, or an area of cultivation. In another embodiment, the plant is stably or transiently transformed directly with a DNA construct or expression cassette or infected by a viral vector for expression of at least one silencing element. However, stable or transiently transformed plants are preferably used for production of small RNAs of the invention, which are then provided to the plant of interest for protection against one or multiple pathogenic bacteria.

The silencing element of the invention can also be applied by trunk injection and petiole absorption, as disclosed in (41).

The phytotherapeutic compositions of the invention can also comprise cells (such as crude plant cell extracts), in which a polynucleotide encoding the iRNA of the invention is stably integrated into the genome and operably linked to active promoters in the cell. Compositions comprising a mixture of cell extracts, some cell extracts from plant cells expressing at least one iRNA of the invention, are also encompassed. In other embodiments, the phytotherapeutic compositions of the invention do not contain any cell.

In one embodiment, the composition is applied externally to a plant (i.e., by spraying a field or area of cultivation) to protect the plant from bacterial infection. In another embodiment, it is applied by trunk injection and petiole absorption.

The phytotherapeutic compositions of the invention can be formulated in an agriculturally suitable and/or environmentally acceptable carrier. Such carriers can be any material that the plant to be treated can tolerate. Furthermore, the carrier must be such that the composition remains effective at controlling the bacteria infection, and not toxic for animals or insects that feed on the treated plants. Examples of such carriers include water, saline, Ringer's solution, dextrose or other sugar solutions, Hank's solution, and other aqueous physiologically balanced salt solutions, phosphate buffer, bicarbonate buffer and Tris buffer.

The compositions of the invention can be supplied in a concentrated form, such as a concentrated aqueous solution. It may even be supplied in frozen form or in freeze-dried or lyophilized powder form. This latter may be more stable for long term storage and may be de-frosted and/or reconstituted with a suitable diluent immediately prior to use.

These compositions may furthermore contain a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaricides, plant growth regulators, harvest aids, and fertilizers, can be combined with carriers, surfactants or adjuvants customarily employed in the art of formulation or other components to facilitate product handling and application for particular target bacteria. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers.

The compositions of the invention can be solid slow release formulations, surfactant diatomaceous earth formulations for pesticidal use in the form of dry spreadable granules, water-insoluble lipospheres formed of a solid hydrophobic core having a layer of a phospholipid embedded on the surface of the core, microcapsules, etc.

The nature of the excipients and the physical form of the composition may vary depending on the nature of the plant part that is desired to treat.

The active ingredients of the present invention (i.e., the silencing element) can normally be applied to the crop area, plant, reproductive organs, fruits, seed and roots to be infected or that is already infected.

Methods of applying an active ingredient or a composition that contains at least one silencing element include, but are not limited to, petiole absorption, foliar application, seed coating, and soil application. The number of applications and the rate of application depend on the intensity of the bacterial infection.

Specifically, the compositions of the invention can be applied to the plants by, for example, spraying, atomizing, dusting, scattering, coating or pouring, introducing into or on the soil, introducing into irrigation water, by seed treatment or general application or dusting at the time when the bacterial infection has begun or before the bacterial infection as a protective measure.

The invention also relates to the use of said phytotherapeutic composition for inhibiting or preventing the growth or pathogenicity of bacteria on target plants.

In other words, the composition of the invention can be used for:
Treating and/or preventing and/or controlling bacterial pathogenicity,
Treating and/or preventing and/or controlling bacterial growth, thereby increasing plant yields.

### Extracellular Vesicles Comprising the Small RNAs of the Invention

In a preferred embodiment, the small RNAs of the invention or their precursors are contained within natural Extracellular Vesicles (EVs) or in artificial vesicles in which they will be protected from the action of RNases. Layered Double Hydroxide (LDH) clay nanosheets, which are non-toxic and degradable, can also be used to carry antibacterial iRNAs. They have already been successfully employed to deliver antiviral dsRNAs and were found to confer viral protection for a period of at least 20 days (42).

A number of studies have now been published, highlighting the important protective role of EVs in the delivery of small RNAs to plant eukaryotic pathogens (17, 19).

The present inventors herein show that the delivery of small RNAs from plant to bacteria also occurred, at least partially, through EVs excreted by the transgenic plants (FIG. 9B).

The compositions of the invention therefore preferably contain EVs that have been secreted by the transgenic plants of the invention.

EVs have heterogeneous size diameters (43, 44). They contain cytosolic and membrane proteins derived from the parental cells (43-46). They also contain functional mRNAs, long non-coding RNAs, miRNA precursors and mature miRNAs and siRNAs (17, 19, 47, 48).

Purification of EVs can be performed by various methods, the most common and most preferred of which being differential ultracentrifugation (43, 44).

More particularly, it is possible to obtain EVs from plant cells by filtration and differential centrifugation steps as previously described (43, 44). Briefly, leaves are vacuum infiltrated with classical buffers used to collect apoplastic wash fluid (e.g. pH 6 MES buffer) and further centrifuged at low speed (44). The apoplastic wash fluid is further collected, filtered and centrifuged successfully as recently described (44). A population of plant EVs, in a size range of approximately 50 to 300 nm in Arabidopsis (with a median at 150 nm) can be recovered at a centrifugation speed of 40,000 g from apoplastic fluid (44). Smaller EVs, in a size range of approximately 10-20 nm in Arabidopsis, can also be recovered by exerting differential ultracentrifugation from apoplastic fluid at centrifugation speed at 40,000 g followed by another one at 100,000 g on the supernatant obtained in the previous step (44). Plant EVs can be also concentrated using dedicated columns (e.g. Amicon Ultra-15 Centrifugal Filters Ultracel 30K), and resuspended in dedicated buffer so that they can be subsequently used for incubation with bacterial cells (in vitro assay) or exogenously applied on plant surface (in planta assay) prior or after bacterial infections.

The composition of the invention may also contain apoplastic EV-free small RNAs secreted by the transgenic plants of the invention and that are not associated with proteins. These small RNA species are referred to here as Extracellular Free Small RNAs or “efsRNAs”. These small RNA species can be obtained by recovering the supernatant from either a differential ultracentrifugation of apoplastic fluid involving a 100,000 g centrifugation speed or the supernatant from a differential ultracentrifugation of apoplastic fluid involving a 40,000 g followed by a 100,000 g centrifugation speed. The resulting supernatant can be mixed in dedicated buffer or used directly for incubation with bacterial cells (in vitro assay) or exogenously applied on plant surface (in planta assay) prior or after bacterial infections.

These EV fractions are advantageously kept or supplied in frozen form or in freeze-dried or lyophilized powder form, under which they maintain their high functionality.

Kit of Parts with Bactericidal Compounds

The compositions may be applied simultaneously or in succession with other compounds.

In a preferred embodiment, the phytotherapeutic composition of the invention contains, in addition to the silencing element of the invention, a bactericidal compound. This is particularly appropriate when the silencing element of the invention inhibits the expression of a gene that triggers the resistance to said bactericidal compound.

In this case, the composition of the invention may be supplied as a “kit of parts”, comprising the silencing element of the invention (the small RNAs defined above) and the corresponding bactericidal compound in a separate container.

Said kit of part preferably contains the phytotherapeutic composition of the invention containing EVs carrying small RNAs and the corresponding bactericidal compound.

The invention also relates to the use of said combination product, for inhibiting or preventing the growth or pathogenicity of bacteria on target plants.

In other terms, the present invention relates to a method for treating target plants against bacterial infection, said method comprising the step of introducing into a cell of said target plant a long dsRNA molecule targeting at least one antibacterial resistance gene, and delivering to said plant the corresponding bactericidal compound.

The invention also relates to a method for treating target plants against bacterial infection, said method comprising the step of delivering small RNAs inhibiting at least one antibacterial resistance bacterial gene, or a composition containing such small RNAs, as well as the corresponding bactericidal compound, on target plant tissues prior to and/or after bacterial infection.

The small RNAs or EVs containing the same are preferably applied prior to the bactericidal compound, for example few hours before, typically, two hours before.

### Screening System of the Invention

In one specific embodiment, the methods of the invention can be also used as tools for experimental research, particularly in the field of functional genomics. Down-regulating bacterial genes with small RNAs can be indeed used to study gene function, in an analogous approach to what has been described in the art for the nematode worm C. elegans and also Drosophila melanogaster. This approach is particularly useful against bacteria that cannot be cultured in vitro.

Assays based on targeted down- or up-regulation of specific bacterial gene(s), leading to a measurable phenotype, provide new tools for identifying anti-bacterial agents.

The Inventors have indeed further developed assays to identify candidate small RNAs having antibacterial activity prior to in planta assays (the latters are more time-consuming for the experimentalist). As demonstrated in FIG. 8C/D, this system can rely on the transient expression of small RNAs using well-established Agrobacterium-mediated transient transformation of tobacco leaves. It can be followed by the incubation of corresponding candidate siRNAs with bacterial cells (in the presence of plant tissues/extracts in the proximity of bacterial cells or in in vitro media such as minimal media mimicking the host environment, which are known to trigger the expression of virulence factors).

In another aspect, the present invention relates to in vitro screening methods allowing the rapid, reliable and cost-effective identification of functional iRNAs having an antibacterial activity, said method comprising the steps of:

a) expressing in plant cells small RNAs inhibiting at least one bacterial gene, collecting the plant extracts or the total RNAs secreted by said plant cells,
b) incubating said plant cells or said small RNAs or said extracts with bacterial cells (grown in in vitro media or in the presence of plant tissues/extracts in their proximity), and
c) assessing the expression/activity of reporters (e.g. reporters of bacterial replication, of general stress response, cell division etc), metabolic activity (e.g. exogenous delivery of the fluorescent marker resazurin that is commonly used to monitor bacterial respiratory activity, redox balance indicator and viability), growth (e.g. expression of fluorescent reporter driven by a constitutive promoter that is either chromosomally integrated or encoded from a plasmid), the expression of the gene that is targeted by small RNAs (e.g. RT-qPCR analysis, Western Blot analyses, expression of a reporter gene fused to the targeted gene or the region of the gene that is targeted by small RNAs) of said bacterial cells.

Stable or transient expression of the antibacterial small RNAs can be used, as disclosed above. For transient expression, said plant cells are preferably tobacco leaves cells that can be easily and efficiently transformed with exogenous constructs through Agrobacterium-mediated transient transformation. All the embodiments proposed above for the production of iRNAs, the vectors, the host cells, the targeted genes, the bacteria and the transformation technics are herewith encompassed and do not need to be repeated.

It is also possible to use the apoplastic fluid of the plant cells, containing the secreted molecules and EVs (in association with the effective small RNAs), to contact the bacterial cells in step b). The apoplastic fluid can be recovered by any conventional means such as vacuum infiltration and centrifugation that are commonly used by those skilled in the art. Concentration of EVs can be also further performed using dedicated columns (e.g. Amicon Ultra-15 Centrifugal Filters Ultracel 30K), according to manufacturer instructions.

The method of the invention may contain a final step d) comparing the viability, growth, metabolic or gene reporter activities of bacterial cells incubated with the said apoplastic fluid or said small RNAs with the ones of the same bacterial cells but in the absence of the apoplastic wash fluid or said small RNAs or, preferentially, in the presence of apoplastic wash fluid—from plants expressing control small RNAs—or control small RNAs targeting unrelated genes such as the fungal genes CYP51 from F. graminearum as used in the present invention.

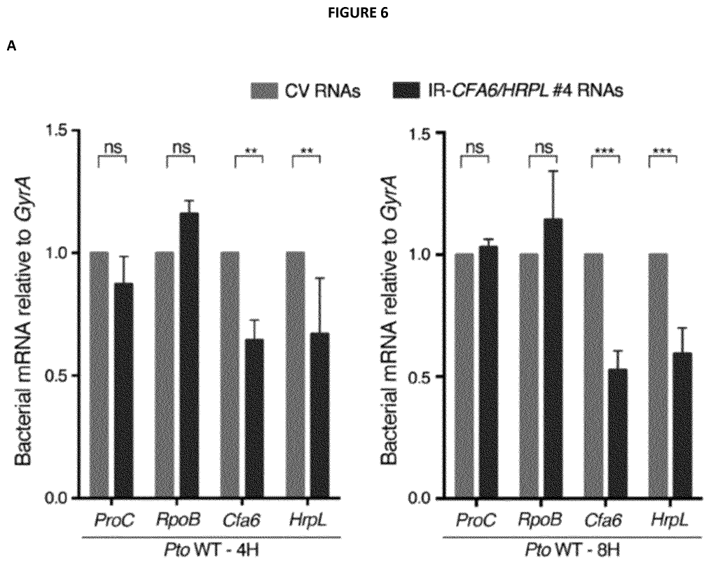

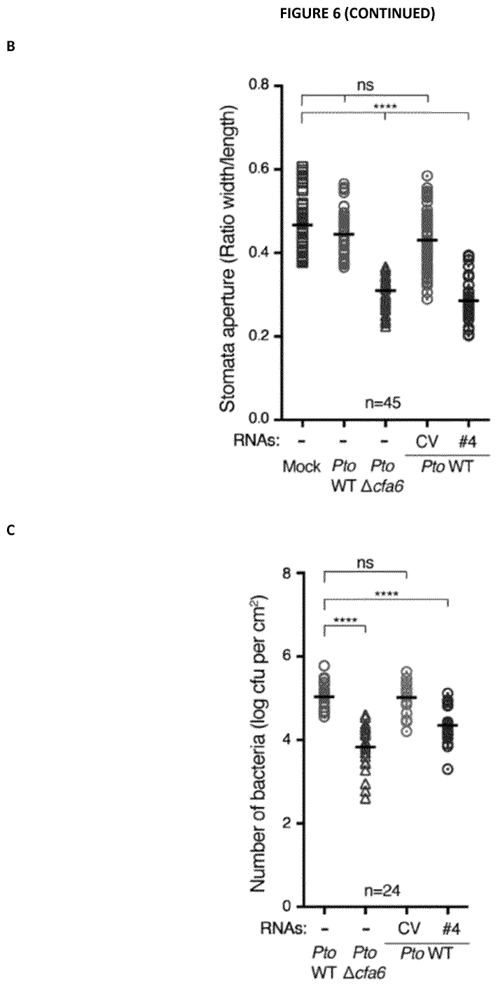

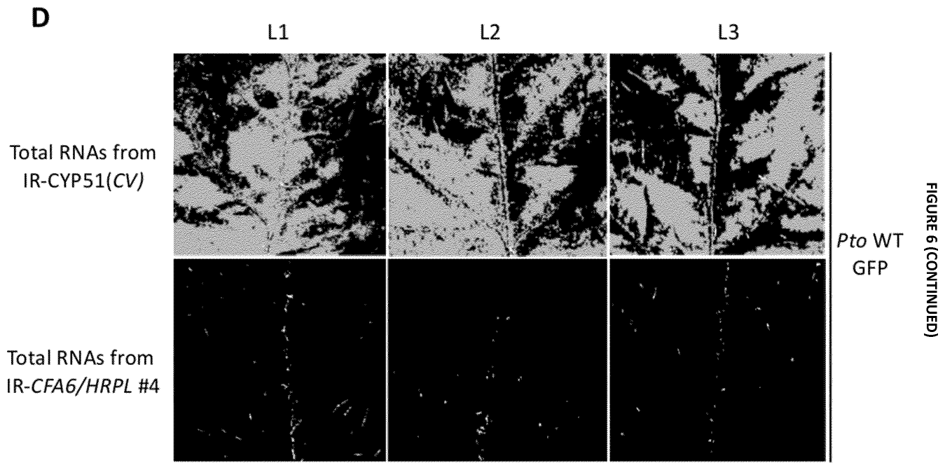

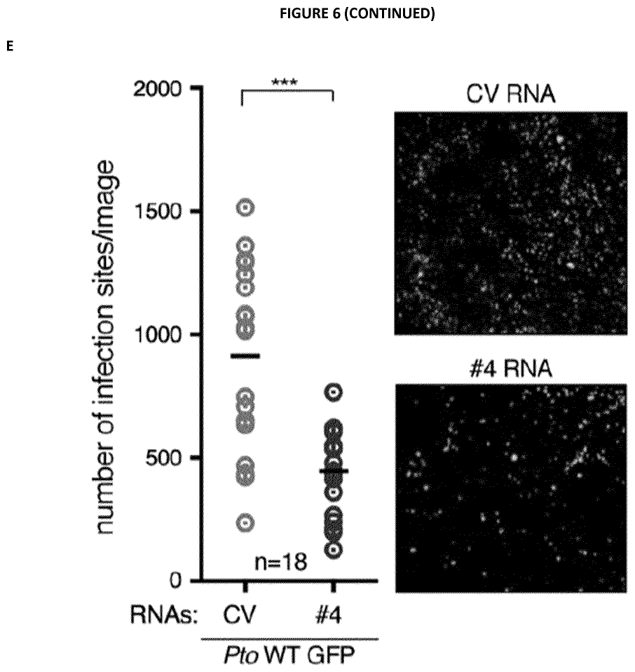

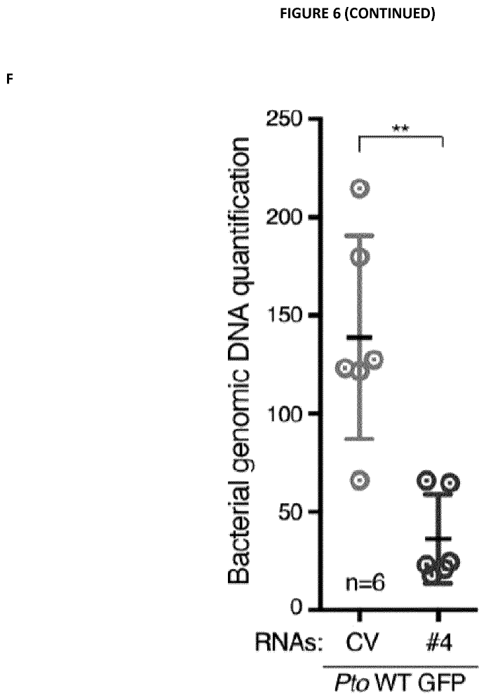

The present inventors also developed systems that are not related to plant production of small RNAs, but instead use rapid in vitro synthesis of double-stranded small RNAs targeting bacterial genes (FIG. 10). As a proof-of-concepts experiments, the inventors have demonstrated that in vitro synthesized anti-Cfa6 and anti-HrpL siRNAs triggered bacterial gene silencing as well as suppression of Pto DC3000-induced stomatal reopening to the same extent as total RNAs derived from IR-CFA6/HRPL transgenic plants (FIG. 10B/C, FIG. 6A). Furthermore, they have shown that in vitro-synthesized anti-fusA and anti-gyrB siRNAs possess a strong bactericidal effect, thereby preventing the growth of Pto DC3000 in in vitro conditions (FIG. 10D/E).

They therefore also propose an in vitro method to identify candidate artificial small RNAs with antibacterial effects, said method comprising the steps of:

a) conducting in vitro synthesis of small RNAs (preferably double-stranded) inhibiting at least one bacterial gene,
b) incubating said small RNAs with bacterial cells, and
c) assessing the viability, growth, metabolic activity, of said bacterial cells, as explained above.

Alternatively, the in vitro screening method of the invention can comprise the steps of:

a) expressing in plant cells at least one long dsRNA inhibiting at least one bacterial gene,
b) contacting said plant cells with a lysis buffer,
c) incubating said plant cell lysates with bacterial cells, and
d) assessing the viability, growth, metabolic activity, of said bacterial cells.

In these screening methods, any bacterial gene can be targeted, the purpose being to identify if said gene is involved in bacterial growth, virulence, pathogenicity, bacterial resistance, etc. Appropriate settings for step c) or d) can be developed by the skilled artisan in order to identify such activity.

It is anticipated that these screening systems will be exploited in the future to select, and eventually produce, efficient antibacterial small RNAs, that can be incorporated into phytotherapeutic compositions or agents in order to protect plants against bacterial diseases in further in planta and field conditions.

They can be coupled with microfluidic device or other screening methods using high throughput automatic proceedings to read the phenotypic behavior in multiplex.

In addition to the above arrangements, the invention also comprises other arrangements, which will emerge from the description that follows, which refers to exemplary embodiments of the subject of the present invention, with reference to the attached drawings and Table of sequences in which:

TABLE I
Sequence details on the tools used in the examples

| SEQ | |
|---|---|
| ID | |
| NO: | Name/details |
| 1 | Sequence of the first arm of the CFA6/HRPL dsRNA used to |
| | concomitantly target HrpL and Cfa6 genes of Pto DC3000 |
| 2 | Sequence of the CHSA intron used to generate all the inverted |
| | repeat from the present invention |
| 3 | Sequence of the second arm of the CFA6/HRPL dsRNA used |
| | to concomitantly target HrpL and Cfa6 genes of Pto DC3000 |
| 4 | Sequence of the first arm of the CFA6-A dsRNA used to target |
| | the Cfa6 gene of Pto DC3000 |
| 5 | Sequence of the second arm of the CFA6-A dsRNA used to |
| | target the Cfa6 gene of Pto DC3000 |
| 6 | Sequence of the first arm of the CFA6-B dsRNA used to |
| | target the Cfa6 gene of Pto DC3000 |
| 7 | Sequence of the second arm of the CFA6-B dsRNA used to |
| | target the Cfa6 gene of Pto DC3000 |
| 8 | Sequence of the first arm of the HRPL-A dsRNA used to |
| | target the HrpL gene of Pto DC3000 |
| 9 | Sequence of the second arm of the HRPL-A dsRNA used to |
| | target the HrpL gene of Pto DC3000 |
| 10 | Sequence of the first arm of the HRPL-B dsRNA used to |
| | target the HrpL gene of Pto DC3000 |
| 11 | Sequence of the second arm of the HRPL-B dsRNA used to |
| | target the HrpL gene of Pto DC3000 |
| 12 | Sequence of the first arm of the HRCC dsRNA used to |
| | target the HrcC gene of Pto DC3000 |
| 13 | Sequence of the second arm of the HRCC dsRNA used to |
| | target the HrcC gene of Pto DC3000 |
| 14 | Sequence of the first arm of the AvrPto/AvrPtoB dsRNA |
| | used to target the AvrPto and AvrPtoB genes of Pto DC3000 |
| 15 | Sequence of the second arm of the AvrPto/AvrPtoB dsRNA |
| | used to target the AvrPto and AvrPtoB genes of Pto DC3000 |
| 16 | Sequence of the first arm of the CYP51 dsRNA used to |
| | target the FgCYP51A, FgCYP51B and FgCYP51C genes |
| | of Fusarium graminearum |
| 17 | Sequence of the second arm of the CYP51 dsRNA used to |
| | target the FgCYP51A, FgCYP51B and FgCYP51C genes |
| | of Fusarium graminearum |
| 18 | Sequence of the first arm of the HRPG/HRPB/HRCC |
| | dsRNA used to target concomitantly the HrpG, HrpB and HrcC |
| | genes of Ralstonia species |
| 19 | Sequence of the second arm of the HRPG/HRPB/HRCC |
| | dsRNA used to target concomitantly the HrpG, HrpB and |
| | HrcC genes of Ralstonia species |
| 20 | Sequence of the first arm of the HRPB/HRCC/TssB/XpsR |
| | dsRNA used to target concomitantly the HrpB, HrcC, XpsR and |
| | TssB genes of Ralstonia species |
| 21 | Sequence of the second arm of the HRPB/HRCC/TssB/XpsR |
| | dsRNA used to target concomitantly the HrpB, HrcC, XpsR |
| | and TssB genes of Ralstonia species |
| 22 | Sequence of the first arm of the HRPG/HRPX/RsmA dsRNA |
| | used to target concomitantly the HrpG, HrpX, and RsmA |
| | genes of Xanthomonas campestris pv. campestris |
| 23 | Sequence of the second arm of the HRPG/HRPX/RsmA |
| | dsRNA used to target concomitantly the HrpG, HrpX, and |
| | RsmA genes of Xanthomonas campestris pv. campestris |
| 24 | Sequence of the first arm of the RpoB/RpoC/FusA dsRNA |
| | used to target concomitantly the RpoB, RpoC and FusA genes |
| | of Pto DC3000 and Pseudomonas syringae CC440 |
| 25 | Sequence of the second arm of the RpoB/RpoC/FusA dsRNA |
| | used to target concomitantly the RpoB, RpoC and FusA genes of |
| | Pto DC3000 and Pseudomonas syringae CC440 |
| 26 | Sequence of the first arm of the SecE/RpoA/RplQ dsRNA |
| | used to target concomitantly the SecE, RpoA and RplQ genes |
| | of Pto DC3000 and Pseudomonas syringae CC440 |
| 27 | Sequence of the second arm of the SecE/RpoA/RplQ dsRNA |
| | used to target concomitantly the SecE, RpoA and RplQ genes of |
| | Pto DC3000 and Pseudomonas syringae CC440 |
| 28 | Sequence of the first arm of the NadHb/NadHd/NadHe |
| | dsRNA used to target concomitantly the NadHb, NadHd and |
| | NadHe genes of Xanthomonas species |
| 29 | Sequence of the second arm of the NadHb/NadHd/NadHe |
| | dsRNA used to target concomitantly the NadHb, NadHd and |
| | NadHe genes of Xanthomonas species |
| 30 | Sequence of the first arm of the DnaA/DnaE1/DnaE2 dsRNA |
| | used to target concomitantly the DnaA, DnaE1 and DnaE2 |
| | genes of Xanthomonas species |
| 31 | Sequence of the second arm of the DnaA/DnaEl /DnaE2 |
| | dsRNA used to target concomitantly the DnaA, DnaE1 and |
| | DnaE2 genes of Xanthomonas species |
| 32 | GFP reporter sequence contained in the GFPpPNpt plasmid |
| 33 | Primer sequence of Cfa6-Forward used for LMW Northern Blot |
| 34 | Primer sequence of Cfa6-Reverse used for LMW Northern Blot |
| 35 | Primer sequence of HrpL-Forward used for LMW Northern Blot |
| 36 | Primer sequence of HrpL-Reverse used for LMW Northern Blot |
| 37 | Primer sequence of miR159 probe used for LMW Northern Blot |
| 38 | Primer sequence of GyrA-Fwd used for RT-qPCR |
| 39 | Primer sequence of GyrA-Rev used for RT-qPCR |
| 40 | Primer sequence of CFA6-Fwd used for RT-qPCR |
| 41 | Primer sequence of CFA 6-Rev used for RT-qPCR |
| 42 | Primer sequence of HrpL-Fwd used for RT-qPCR |
| 43 | Primer sequence of HrpL-Rev used for RT-qPCR |
| 44 | Primer sequence of ProC-Fwd used for RT-qPCR |
| 45 | Primer sequence of ProC-Rev used for RT-qPCR |
| 46 | Primer sequence of RpoB-Fwd used for RT-qPCR |
| 47 | Primer sequence of RpoB-Rev used for RT-qPCR |
| 48 | Primer sequence of Cyp3-Fwd used for LMW Northern Blot |
| 49 | Primer sequence of Cyp3-Rev used for LMW Northern Blot |
| 50 | Probe preparation for northern blot analysis: U6 |
| 51 | Primer sequence of Tomato Ubi-Fwd used for RT-qPCR |
| 52 | Primer sequence of Tomato Ubi-Rev used for RT-qPCR |
| 53 | Primer sequence of Pto GFP-Fwd used for RT-qPCR |
| 54 | Primer sequence of Pto GFP-Rev used for RT-qPCR |
| 55 | Primer sequence of IR-CFA6/HRPL-Fwd used for RT-qPCR |
| 56 | Primer sequence of IR-CFA6/HRPL-Rev used for RT-qPCR |
| 57 | Primer sequence of Ath-Ubi-Fwd used for RT-qPCR |
| 58 | Primer sequence of Ath-Ubi-Rev used for RT-qPCR |
| 59 | HRPL-pDON207-Fwd for Cloning of WT HRPL and mut |
| | HRPL in pDON207-attB1/B2 |
| 60 | HRPL-pDON207-Rev for Cloning of WT HRPL and mut |
| | HRPL in pDON207-attB1/B2 |
| 61 | Primer dcl2-1-WT-fwd for genotyping dcl2-1 allele |
| 62 | Primer dcl2-1-mut-fwd for genotyping dcl2-1 allele |
| 63 | Primer dcl2-1-WT-Rev for genotyping dcl2-1 allele |
| 64 | Primer dcl3-1-fwd for genotyping dcl3-1 allele |
| 65 | Primer dcl3-1-Rev for genotyping dcl3-1 allele |
| 66 | Primer LBal |
| 67 | Primer dcl4-2-G8605 Fwd for genotyping dcl4-2 allele |
| 68 | Primer dcl4-2-G8605 Rev for genotyping dcl4-2 allele |
| 69 | Primer GABI-8474-LP |
| 70 | Northern blot analysis IR-HHR Fwd Primer |
| 71 | Northern blot analysis IR-HHR Rev Primer |
| 72 | RT-qPCR LuxA Fwd |
| 73 | RT-qPCR LuxA Rev |
| 74 | RT-qPCR LuxB Fwd |
| 75 | RT-qPCR LuxB Rev |
| 76 | HRPL-pDON207-Fwd |
| 77 | HRPL-pDON207-Rev |
| 78 | T7 Fwd CFA6/HRPL |
| 79 | T7 Rev CFA6/HRPL |
| 80 | T7 Fwd CYP51 |
| 81 | T7 Rev CYP51 |
| 82 | T7 Fwd Dc3000_FusA |
| 83 | T7 Rev Dc3000_FusA |
| 84 | T7 Fwd Dc3000_SecE |
| 85 | T7 Rev Dc3000_SecE |
| 86 | T7 Fwd Dc3000_GyrB |
| 87 | T7 Rev Dc3000_GyrB |
| 88 | First strand XC_RS06155: XC_1225 |
| 89 | Second strand XC_RS06155: XC_1225 |
| 90 | First strand XC_RS02265 = XC_0447 |
| 91 | Second strand XC_RS02265 = XC_0447 |
| 92 | First strand XC_RS18260 = XC_3609 |
| 93 | Second strand XC_RS18260 = XC_3609 |
| 94 | First strand XC_RS11930 = XC_2375 |
| 95 | Second strand XC_RS11930 = XC_2375 |
| 96 | First strand XC_RS17005 = XC_3357 |
| 97 | Second strand XC_RS17005 = XC_3357 |
| 98 | T7 Fwd XC_RS06155: XC_1225 |
| 99 | T7 Rev XC_RS06155: XC_1225 |
| 100 | T7 Fwd XC_RS02265 = XC_0447 |
| 101 | T7 Rev XC_RS02265 = XC_0447 |
| 102 | T7 Fwd XC_RS18260 = XC_3609 |
| 103 | T7 Rev XC_RS18260 = XC_3609 |
| 104 | T7 Fwd XC_RS11930 = XC_2375 |
| 105 | T7 Rev XC_RS11930 = XC_2375 |
| 106 | T7 Fwd XC_R517005 = XC_3357 |
| 107 | T7 Rev XC_RS17005 = XC_3357 |
| 108 | Sequence of the first arm of the LuxA/LuxB dsRNA used to |
| | target concomitantly the LuxA and LuxB genes of Pto DC3000 |
| 109 | Sequence of the second arm of the LuxA/LuxB dsRNA used |
| | to target concomitantly the LuxA and LuxB genes of Pto |
| | DC3000 |

### FIGURE LEGENDS

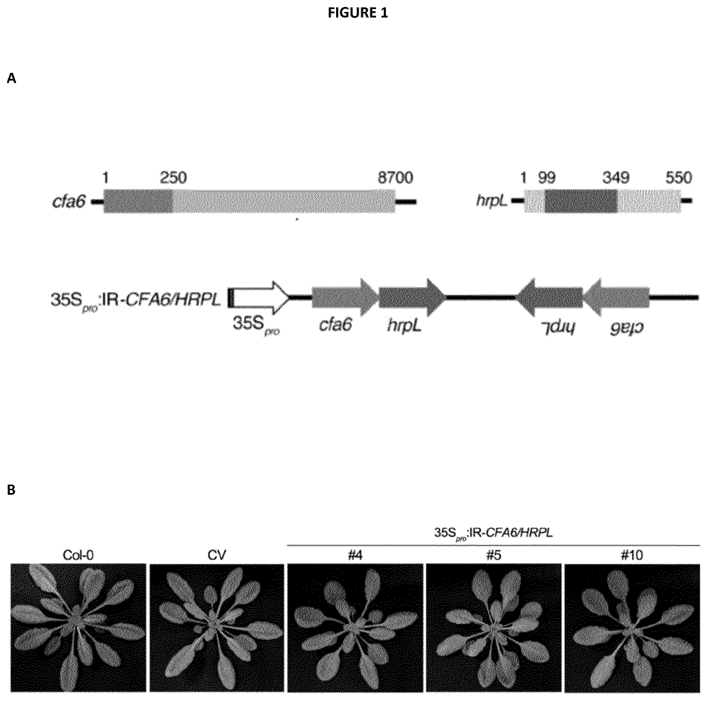

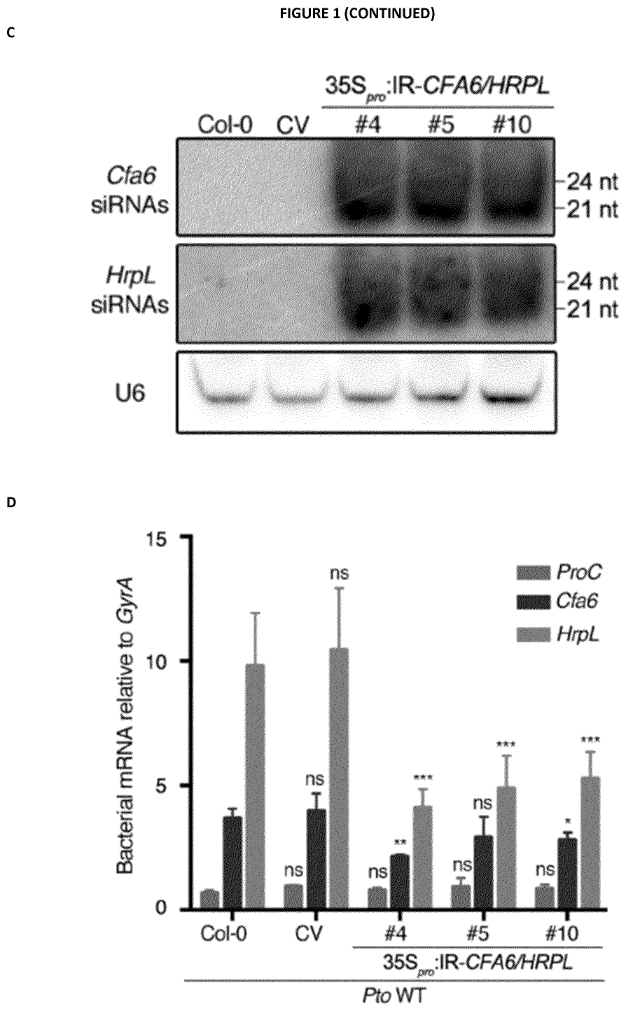

FIG. 1. Phenotypical and molecular characterization of Arabidopsis transgenic plants expressing the inverted repeat IR-CFA6/HRPL in both untreated and bacterial challenged conditions
A. Schematic representation of the Pto DC3000 genes Cfa6 and HrpL. The 250 bp regions of Cfa6 (1-250 nt) and HrpL (99-348 nt) genes were used to generate the chimeric hairpin construct under the control of the constitutive 35S promoter.
B. Representative pictures of five-week old Col-0 plants and of independent homozygous transgenic plants expressing the 35 Sₚᵣₒ:IR-CYP51 (Control vector: CV) or the 35Sₚᵣₒ:IR-CFA6/HRPL construct.
C. Accumulation level of anti-Cfa6 and anti-HrpL siRNAs detected by low molecular weight Northern blot analysis of the Arabidopsis plants depicted in B. U6 was used as a loading control.
D. Pto DC3000 HrpL mRNA accumulation is significantly decreased on IR-CFA6/HRPL-infected plants compared to Col-0- and CV-infected plants. Arabidopsis plants depicted in B. were dip-inoculated with Pto DC3000 WT strain and at 3 days post-infection (dpi), bacterial transcript levels of ProC, Cfa6 and HrpL were monitored by quantitative RT-PCR analysis. These mRNA levels are quantified relative to the level of bacterial GyrA transcript. Error bars indicate the standard deviations of mRNA values obtained in three independent experiments. Statistically significant differences were assessed using ANOVA test (ns: p-value>0.05; *: p-value<0.05, **: p-value<0.01, ***: p-value<0.001).

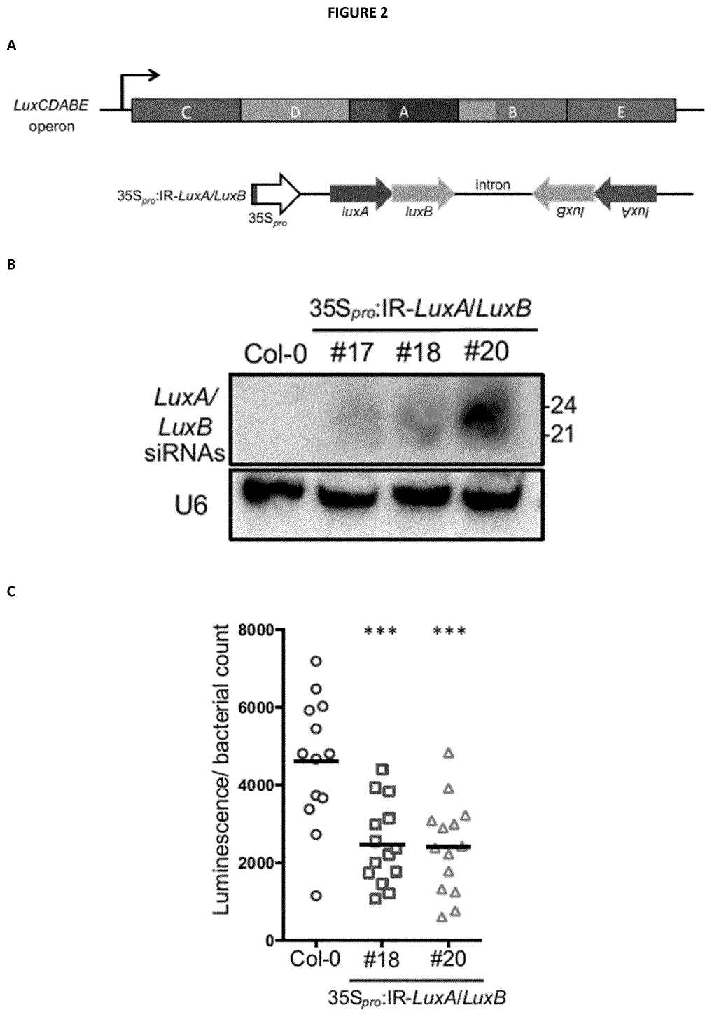

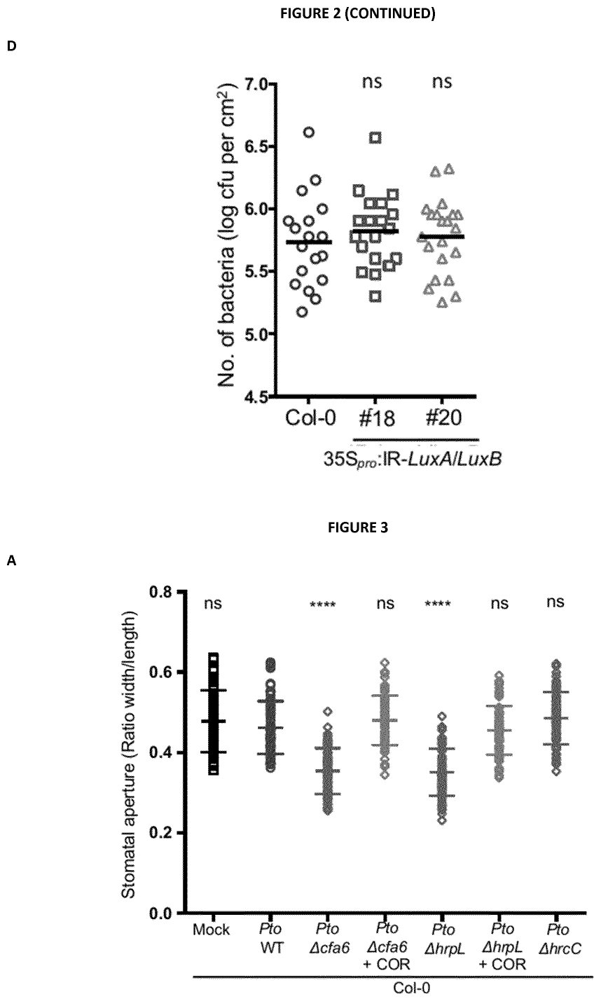

FIG. 2. Phenotypical and molecular characterization of Arabidopsis transgenic plants expressing the inverted repeat IR-LuxA/LuxB in both untreated and bacterial challenged conditions
A. Schematic representation of the luxCDABE operon inserted into Pto DC3000 WT genome. The 250 bp regions of LuxA (1-250 nt) and LuxB (1-250 nt) genes were used to generate the chimeric hairpin construct under the control of the constitutive 35S promoter.
B. Accumulation level of anti-LuxA/LuxB detected by low molecular weight Northern blot analysis of the Arabidopsis transgenic plants. U6 was used as a loading control.
C. A significant impact on the luminescence of Pto DC3000 luciferase (Pto Luc) was observed in the transgenic lines expressing the IR-LuxA/LuxB as compared to Col-0 upon infection. The two independent transgenic lines of IR-LuxA/LuxB #18 and #20, along with Col-0 were syringe-infiltrated with Pto Luc at a concentration of 10⁶ cfu/ml and the luminescence was measured at 24 hours-post infiltration.
D. The in planta growth of Pto DC3000 is unaltered in IR-LuxA/LuxB transgenic plants compared to Col-0 plants. Leaf discs from the plants used in C. were grinded and plated in a serial dilution to count Pto Luc for each condition at 24 hours post-infection.

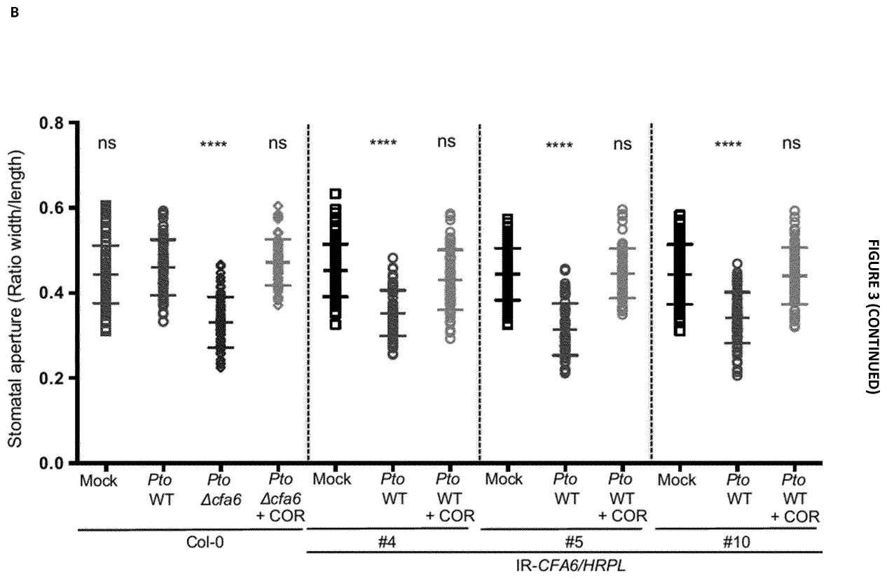

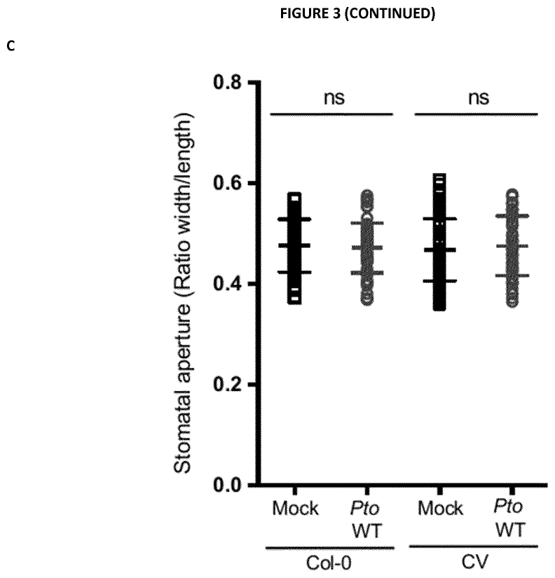

FIG. 3. Arabidopsis transgenic plants expressing the IR-CFA6/HRPL construct suppress Pto DC3000-induced stomatal reopening
A. The Pto Δcfa6 and ΔhrpL strains, but not the ΔhrcC strain, were impaired in their ability to reopen stomata and these phenotypes were rescued upon addition of exogenous COR. Sections of unpeeled leaves of Col-0 plants were incubated with mock solution (water) or Pto DC3000 WT, Δcfa6, ΔhrpL or ΔhrcC strains for 3 hours. Stomata aperture was assessed by measuring the width and length using ImageJ software.
B. Pto DC3000 WT no longer induced stomatal reopening in Arabidopsis transgenic lines overexpressing the IR-CFA6/HRPL hairpin. Stomatal aperture measurement was conducted in Col-0 and 35Sₚro:IR-CFA6/HRPL #4, #5, #10 transgenic lines infected with Pto WT strain as described in A.
C. The Pto DC3000-induced stomatal reopening response was unaltered in CV compared to Col-0 plants. Stomatal aperture measurement was conducted in Col-0 and CV plants infected with Pto WT strain as described in A.
   Note: For all these experiments, n=number of stomata analyzed per condition and statistical significance was assessed using the ANOVA test (ns: p-value >0.05; ****: p-value <0.0001).

FIG. 4. Arabidopsis transgenic plants expressing the IR-CFA6/HRPL construct exhibit a reduced vascular spreading and growth of Pto DC3000 in adult leaves
A. IR-CFA6/HRPL #4, #5 and #10-infected plants exhibit reduced vascular spreading of Pto WT compared to Col-0- and CV-infected plants. Plants were wound-inoculated in midveins with Pto WT-GFP and Col-0 was wound-inoculated with PtoΔcfa6-GFP. GFP fluorescence signal was observed under UV light and pictures were taken at 3 days post-infection (dpi). To index the spreading of bacteria from the inoculation sites, GFP fluorescence was observed under UV light. When the bacteria propagated away from any of the three inoculation sites, it was indexed as propagation with 4 corresponding to the highest propagation index. Pictures from three biological replicates were taken into consideration.
B. Representative picture of infected leaves of conditions used in A. are depicted. White circles indicate the site of wound-inoculation in the leaf midvein.
C. IR-CFA6/HRPL #4, #5 and #10 transgenic lines exhibit a significantly reduced Pto WT titer when compared to Col-0 and CV-infected plants. Col-0, CV and IR-CFA6/HRPL #4, #5 and #10 plants were dip-inoculated with Pto WT and Col-0 plants were dip-inoculated with the PtoΔcfa6-GFP strain. Bacterial titers were monitored at 2 days post-infection (dpi). Four leaves from three plants per condition and from three independent experiments (n) were considered for the comparative analysis.
D. IR-CFA6/HRPL #4, #5 and #10 transgenic plants exhibit reduced water-soaking symptoms in comparison to Col-0 and CV plants. Representative leaf pictures of water-soaking symptoms were taken 24 hours after dip-inoculation.
   Note: For all the above experiments, statistical significance was assessed using the two-way ANOVA test (ns: p-value>0.05; *: p-value<0.05; **: p-value<0.01; ***: p-value<0.001; ****: p-value<0.0001).

FIG. 5. Phenotypical characterization of Arabidopsis transgenic plants expressing the inverted repeat IR-HRPG/HRPX/RSMA in both untreated and Xanthomonas campestris pv. campestris challenged conditions
A. IR-HRPG/HRPX/RSMA #1- and #6-infected plants exhibit reduced vascular spreading of the virulent XccΔXopAC (GUS/GFP) strain compared to Col-O-infected plants. Plants were wound-inoculated in midveins with XccΔXopAC (GUS/GFP) at OD=0.01. GFP fluorescence signal was observed under UV light and pictures were taken at 3 days post-infection (dpi). The indexing was done as described in 4A.
B. Representative picture of infected leaves of conditions used in B. are depicted. White circles indicate the site of wound-inoculation in the leaf midvein.

FIG. 6. Exogenously delivered total RNAs from IR-CFA6/HRPL transgenic plants reduce Pto DC3000 pathogenicity when applied on the surface of wild type Arabidopsis and tomato leaves
A. In vitro AGS assay showing that total RNA extract from CFA6/HRPL #4 plants triggers silencing of both Cfa6 and HrpL genes. Pto WT cells were incubated in vitro for 4 and 8 hours with 20 ng/μl of total RNAs from CV or IR-CFA6/HRPL #4 plants. Significant reduction of the bacterial transcripts Cfa6 and HrpL was observed by RT-qPCR at both the timepoints, while accumulation of ProC and RpoB transcripts remained unaffected. GyrA was used as an internal control to quantify the accumulation of bacterial transcripts. Error bars indicate the standard deviations of values from three independent experiments.
B. The ability of Pto WT to reopen stomata was altered upon exogenous application of total RNAs extract from IR-CFA6/HRPL plants compared to CV plants. Col-0 leaves were treated for 1 hour with water or 20 ng/μl of total RNAs extracted from CV or IR-CFA6/HRPL #4 plants and were incubated with Pto WT for 3 hours. Stomatal aperture was measured and analyzed as described in FIG. 3A.
C. Treatment with IR-CFA6/HRPL, but not with CV, total RNAs compromised the ability of Pto DC3000 to multiply in the apoplast of leaves when compared to pretreatment with CV total RNAs. Col-0 leaves were treated with 20 ng/μl of total RNAs from CV or IR-CFA6/HRPL #4 plants for 1 hour, followed by dip-inoculation with Pto WT. Bacterial titers were monitored at 2 dpi. The number of leaves (n) corresponds to collective values from three independent experiments.
D. The leaves treated with CV total RNAs displayed more necrotic symptoms as compared to the leaves treated with IR-CFA6/HRPL #4 total RNAs. The experiment was conducted as in C. but using five-week-old tomato (Solanum lycopersicum ‘Moneymaker’) plants. Representative pictures of infected leaves in the two conditions are depicted.
E. A reduced number of Pto DC3000-GFP foci was observed in tomato leaves treated with total RNA extracts from IR-CFA6/HRPL #4 versus CV plants. Infected-leaves were observed at 3 dpi under UV light to estimate the number of GFP loci. On the left: Dot plot representing the number of GFP loci analyzed using ImageJ software from 3-4 different leaves per condition with at least 4 pictures per leaf. The values used for the analysis are from two different independent experiments. Student's t-test was performed for the comparative analysis. On the right: Representative picture of the tomato leaves described in D.
F. Pto WT-GFP DNA content is decreased in tomato leaves treated with total RNA extracts from IR-CFA6/HRPL #4 versus CV plants. The level of bacterial DNA content was analyzed by qPCR using tomato Ubiquitin as a control. Student's t-test was performed for the comparative analysis.
   Note: For A, B and C, statistically significant differences were assessed using ANOVA test (ns: p-value>0.05; **: p-value<0.01, ***: p-value<0.001).

FIG. 7. DCL-dependent antibacterial siRNAs, but not corresponding unprocessed dsRNA precursors, are the RNA entities responsible for AGS and for the suppression of stomatal reopening
A. Upper panel: Accumulation level of IR-CFA6/HRPL transcripts in Col-0, dcl2-1 dcl3-1 dcl4-2 (dcl234), IR-CFA6/HRPL #4 (#4) and IR-CFA6/HRPL #4 in dcl234 mutant background (#4×dcl234) was performed by RT-qPCR. Ubiquitin was used as a control. The graph represents the mean and standard deviation of three independent experiments. Lower panel: Accumulation level of anti-Cfa6 and anti-HrpL siRNAs was performed by low molecular weight Northern blot analyses in the same genotypes. U6 was used as a loading control.
B. Total RNA extract from #4×dcl234 plants does not alter the transcript accumulation levels of Cfa6 and HrpL. Pto WT cells were incubated in vitro for 8 hours with 20 ng/μl of total RNAs extracted from the same genotypes described in A. Accumulation levels of Cfa6 and HrpL transcripts was assessed by RT-qPCR analysis using GyrA as a control. Error bars indicate the standard deviations of values from three independent experiments. Statistically significant differences were assessed using ANOVA test (ns: p-value>0.05; *: p-value<0.05, **: p-value<0.01).
C. Total RNA extract from #4×dcl234 plants does not suppress Pto DC3000-induced stomatal reopening response. Col-0 leaves were treated with water or 20 ng/μl of total RNA extracts from the same genotypes than the ones used in A. for 1 hour and incubated with Pto WT for 3 hours. Stomatal aperture was measured and analyzed as described in FIG. 2A. Two other biological replicates are presented in Supplementary FIG. 4B.
D. Upper panel: Electrogram profiles representing the RNA size distribution of total, long and small RNAs from IR-CFA6/HRPL #4 plants determined with an agilent Bioanalyzer 2100 equipped with an RNA Nano chip. Low molecular weight RNA fractions are encircled for each sample. 18S and 25S ribosomal peaks are highlighted. Lower panel: Agarose gel picture of ethidium bromide stained total, long and small RNAs used in A.
E. Small RNA species, but not the corresponding long RNA species, from IR-CFA6/HRPL plants suppress stomatal reopening to the same extent as total RNA extracts. The experiment was conducted as in D. but with total, long (>200 nt) or small (<200 nt) RNA fractions, which were separated from total RNAs of IR-CFA6/HRPL #4 plants.
   Note: For all the stomata experiments, statistical significance was assessed using the ANOVA test (ns: p-value>0.05; ****: p-value<0.0001).

FIG. 8. A bacterially expressed small RNA resilient version of HrpL is refractory to gene silencing directed by anti HrpL siRNAs and exhibits a normal stomatal reopening phenotype upon exogenous application of anti HrpL siRNAs
A. Schematic representation of the PtaΔhrpL strain along with the complementation strains generated upon transformation with the plasmids encoding WT HrpL or mut HrpL, respectively under the control of the constitutive promoter NptII.
B. In vitro AGS assay showing that the PtaΔhrpL WT HrpL strain is sensitive to antibacterial RNAs while the PtaΔhrpL mut HrpL is refractory to these RNA entities. Bacterial PtaΔhrpL WT HrpL and PtaΔhrpL mut HrpL strains were incubated with total RNAs extracted from CV or IR-CFA6/HRPL #4 plants for 8 hours. Accumulation level of WT HrpL and mut HrpL transcripts was analyzed by RT-qPCR (the mRNA levels were relative to the level of GyrA transcript). Error bars indicate the standard deviations of values from three independent experiments. Statistically significant differences were assessed using ANOVA test (ns: p-value>0.05; *: p-value<0.05, **: p-value<0.01).
C. Accumulation of anti-Cfa6 and anti-HrpL siRNAs was assessed by low molecular weight northern analysis using total RNA extracts from N. benthamiana plants transiently expressing 35Sₚᵣₒ:IR-HRPL, 35Sₚᵣₒ:IR-CFA6/HRPL and from non-transformed N. benthamiana leaves (Nb). U6 was used as a loading control.
D. The PtaΔhrpL mut HrpL strain is refractory to anti HrpL siRNA action. Col-0 leaves were treated with total RNAs extracted either from N. benthamiana alone or from N. benthamiana expressing the inverted repeat IR-HRPL. Stomatal reopening response was assessed as described previously.
   Note: For all the stomata experiments, statistical significance was assessed using the ANOVA test (ns: p-value>0.05; ****: p-value<0.0001).

FIG. 9. The apoplastic fluid of IR-CFA6/HRPL plants is composed of functional antibacterial siRNAs that are either embedded into EVs, and protected from micrococcal nuclease action, or in a free form, and sensitive to micrococcal nuclease digestion
A. The ability of Pto WT to reopen stomata was also altered to similar levels upon exogenous application of Apoplastic fluid (APF) extract as compared to total RNAs derived from IR-CFA6/HRPL plants. Total RNAs and APF extracted from CV plants was used as negative control. Col-0 leaves were treated for 1 hour with water (Mock) or 20 ng/μl of total RNAs or 500 μl of APF extracted from CV or IR-CFA6/HRPL #4 plants and were incubated with Pto WT for 3 hours. Stomatal aperture was measured and analyzed as described in previous experiments.
B. The two different vesicular fractions, P40 and P100, as well as the free RNA population present in the supernatent (SN) carry the antibacterial siRNAs and thus are involved in AGS. Apoplastic fluid extracted from both CV and IR-CFA6/HRPL #4 plants was subjected to ultracentrifugation at 40,000 g to pellet the larger population of EVs (P40) and the remaining supernatent was further subjected to ultracentrifugation at 100,000 g to pellet the smaller EVs (P100). SN was also restored. Col-0 leaves were treated for 1 hour with water (Mock) or P40, P100 and SN extracted from CV or IR-CFA6/HRPL #4 plants and were incubated with Pto WT for 3 hours. The P40, P100 and SN of #4 were treated with 20 units of Mnase and the SN of #4 was also treated with 20 units of Proteinase K. Stomatal aperture was measured and analyzed as described in previous experiments.
   Note: For all the stomata experiments, statistical significance was assessed using the ANOVA test (ns: p-value>0.05; ****: p-value<0.0001).

FIG. 10. Exogenous delivery of in vitro synthesized antibacterial siRNAs reduces the pathogenicity as well as the viability of Pto DC3000
A. 2% Agarose gel of ethidium bromide stained in vitro synthesized long dsRNAs and RNase III digested siRNAs corresponding to IR-CYP51 and IR-CFA6/HRPL are depicted.
B. The ability of Pto WT to reopen stomata was altered upon exogenous application of in vitro synthesized siRNAs, but not the long dsRNAs, corresponding to IR-CFA6/HRPL. Long dsRNAs and siRNAs from IR-CYP51 was used as negative control. Col-0 leaves were treated for 1 hour with water (Mock) or RNA presented in A. and then incubated with Pto WT for 3 hours. Stomatal aperture was measured and analyzed as described in previous experiments.
C. In vitro AGS assay using the in vitro synthesized siRNAs from IR-CFA6/HRPL triggers silencing of both Cfa6 and HrpL genes. Pto WT cells were incubated in vitro for 8 hours with 2 ng/μl of in vitro synthesized siRNAs from IR-CYP51 or IR-CFA6/HRPL #4 plants. Significant reduction of the bacterial transcripts Cfa6 and HrpL was observed by RT-qPCR, while accumulation of ProC and RpoB transcripts remained unaffected. GyrA was used as an internal control to quantify the accumulation of bacterial transcripts. Error bars indicate the standard deviations of values from three independent experiments.
D. and E. In vitro synthesized siRNAs against fusA or gyrB of Pto DC3000 have a significant impact on the growth of the Pto DC3000-GFP strain. siRNAs directed against secE, gyrB and fusA genes of Pto DC3000 were synthesized using in vitro transcription followed by RNaseIII digestion. The Pto DC3000-GFP strain was incubated with the indicated concentration of in vitro synthesized siRNAs. 96-well plate was set on the machine for the samples to be fractioned in droplets by the droplet-based microfluidic system (Millidrop). For each well, 10 droplets of ˜500 nl each were formed and incubated inside the instrument. For each droplet, measurements of biomass and of GFP fluorescence were acquired every ˜30 minutes.

### EXAMPLES

### Example 1: Materials and Methods

### Generation of Transgenic Lines Carrying Inverted Repeats Constructs

The IR-CFA6/HRPL chimeric hairpin was designed to produce artificial siRNAs targeting a 250 bp region of Cfa6 (from nucleotide 1 to 250) and a 250 bp region of HrpL from nucleotide 99 to 348 (SEQ ID NO: 1, 2 and 3). The IR-CFA6-A and IR-CFA6-B are two independent inverted repeats that specifically target the Cfa6 gene from nucleotide 1 to 250 (SEQ ID NO: 4, 2 and 5) and from nucleotide 1 to 472 (SEQ ID NO: 6, 2 and 7), respectively. The IR-HRPL-A and IR-HRPL-B are two independent inverted repeats that specifically target HrpL from nucleotide 99 to 348 (SEQ ID NO: 8, 2 and 9) and from nucleotide 1 to 348 (SEQ ID NO: 10, 2 and 11), respectively. The IR-HRCC hairpin was designed to specifically target the HrcC gene (SEQ ID NO: 12, 2 and 13) and the IR-AvrPto/AvrPtoB to concomitantly target the type III effector AvrPto and AvrPtoB genes (SEQ ID NO: 14, 2 and 15). The IR-CYP51 hairpin was designed to produce siRNAs against three cytochrome P450 lanosterol C-14α-demethylase genes of the fungus F. graminearum, namely FgCYP51A, FgCYP51B and FgCYP51C as previously performed (SEQ ID NO: 16, 2 and 17), (19). This hairpin was used as a negative control for all the in planta assays of the invention. Additional inverted repeats were designed and cloned as part of this study to target virulence factors or essential genes from different strains of Pseudomonas, Xanthomonas and Ralstonia. These hairpins are described as follows: the IR-HrpG/HrpB/HrcC hairpin designed to concomitantly target the HrpG, HrpB and HrcC genes from Ralstonia species (SEQ ID NO: 18, 2 and 19), the IR-HrpB/HrcC/TssB/XpsR hairpin designed to concomitantly target the HrpB, HrcC, TssB and XpsR genes from Ralstonia species (SEQ ID NO: 20, 2 and 21), the IR-HrpG/HrpX/RsmA hairpin designed to concomitantly target the HrpG, HrpX and Rsma genes from Xanthomonas campestris pv. campestris (SEQ ID NO: 22, 2 and 23), the IR-RpoB/RpoC/FusA hairpin designed to concomitantly target the essential genes RpoB, RpoC and FusA from Pto DC3000 and Pseudomonas syringae strain CC440 (SEQ ID NO: 24, 2 and 25), the IR-SecE-RpoA-RplQ hairpin designed to concomitantly target the essential genes SecE, RpoA and RplQ from Pto DC3000 and Pseudomonas syringae strain CC440 (SEQ ID NO: 26, 2 and 27), the IR-NadHb/NadHd/NadHe hairpin designed to concomitantly target the essential genes NadHb, NadHd and NadHe from different Xanthomonas species including Xanthomonas campestris pv. campestris (SEQ ID NO: 28, 2 and 29), the IR-DnaA/DnaE1/DnaE2 hairpin designed to concomitantly target the essential genes NadHb, NadHd and NadHe from different Xanthomonas species including Xanthomonas campestris pv. campestris (SEQ ID NO: 30, 2 and 31). Furthermore, a chimeric inverted repeat was designed and cloned as part of this study to target the Photorhabdus luminescens luxCDABE operon chromosomally expressed in Pto DC3000 under the constitutive kanamycin promoter: the IR-LuxA/LuxB hairpin, designed to concomitantly target the LuxA and LuxB genes from Pto DC3000 luciferase strain (SEQ ID NO: 108, 2 and 109). All the above-described hairpins contain a specific intron sequence from the Petunia Chalcone synthase gene CHSA (SEQ ID NO: 2) and were cloned into a vector carrying the Cauliflower Mosaic Virus (CaMV) 35S constitutive promoter. More specifically, the following hairpin sequences: IR-CFA6/HRPL, IR-CYP51, IR-CFA6-B, IR-HRPL-B, IR-HrpG/HrpB/HrcC, IR-HrpB/HrcC/TssB/XpsR, IR-AvrPto/AvrPtoB, IR-HRCC, IR-HrpG/HrpX/RsmA and IR-LuxA/LuxB were cloned into a modified pDON221-P5-P2 vector carrying additional EcoRI and SalI restriction sites to facilitate the insertion of these long inverted-repeats into this vector. A double recombination between pDON221-P5-P2 carrying the hairpin sequence and pDON221-P1-P5r (Life Technologies, 12537-32), carrying the constitutive 35S promoter sequence, was conducted in the pB7WG GATEWAY compatible destination vector (binary vector carrying a BAR selection marker and gateway recombination sites). The remaining hairpins, namely the IR-CFA6-A, IR-HRPL-A, IR-RpoB/RpoC/FusA, IR-SecE-RpoA-RplQ, IR-NadHb/NadHd/NadHe and IR-DnaA/DnaE1/DnaE2 sequences were generated by PCR amplifications of the sense and antisense regions of the target genes using the bacterial genomic DNA as template and followed by the generation of modules required for the cloning into a final GreenGate destination vector pGGZ003. All the plasmids were then introduced into the Agrobacterium tumefaciens strains GV3101 or C58C1 and further used for either transient expression in Nicotiana benthamiana or stable expression in the Arabidopsis thaliana Columbia-0 (Col-0) reference accession.

### Plant Material and Growth Conditions

Stable transgenic lines of IR-CFA6/HRPL and CV were generated by transforming Arabidopsis WT (accession Col-0) plants using Agrobacterium mediated-floral dip method. Three independent transgenic lines, #4, #5 and #10 expressing equal amount of anti-Cfa6 and anti-HrpL siRNAs were selected and propagated until T4 generation. Similarly, selected homozygous line of CV expresses abundant level of siRNAs against F. graminearum CYP51A/B/C genes was propagated until T4 generation for experimentation. Similarly, transgenic lines expressing IR-LuxA/LuxB and IR-HrpG/HrpX/RsmA were selected on the basis of siRNA production and propagated further. For genetic analysis, dcl2 dcl3 dcl4 (dcl234) triple mutant plant was crossed with the reference IR-CFA6/HRPL #4 line and the F3 plants were genotyped to select homozygous dcl234 mutant containing homozygous IR-CFA6/HRPL transgene. Sterilized seeds of Arabidopsis Col-0 and the selected homozygous transgenic lines were first grown for 12-14 days at 22° C. on plates containing ½×MS medium (Duchefa), 1% sucrose and 0.8% agar (with or without antibiotic selection) in 8 h photoperiod. Seedlings were then pricked out to soil pots and grown in environmentally controlled conditions at 22° C./19° C. with an 8 h photoperiod under light intensity of 100 μE/m²/s. Four- to five-week-old plants were used for all the experiments. Seeds of tomato (Solanum lycopersicum ‘Moneymaker’) and N. benthamiana were directly sown on soil pots and grown in environmentally controlled conditions at 22° C./19° C. (day/night) with a 16 h photoperiod under light intensity of 100 μE/m²/s. Four- to five-week old plants were used for all the experiments.

### Bacterial Strains

The GFP expressing Pto DC3000-GFP and the Pto DC3000Δcfa6-GFP (Pto DC3118) strains were a gift from Dr. S. Y. He, while the Pto DC3000ΔhrpL strain was a gift from Dr. Cayo Ramos. The Pto DC3000 luciferase strain was a gift from Dr. Chris Lamb. The Pto DC3000ΔhrpL and Pto DC3000ΔhrcC strains expressing the GFP reporter gene were generated by transforming them with the same plasmid as in Pto DC3000-GFP by electroporation and then plated at 28° C. on NYGB medium (5 g/l bactopeptone, 3 g/l yeast extract, 20 ml/l glycerol) containing gentamycin (1 μg/ml) for selection. To generate the Pto DC3000-WT-HrpL and -mut-HrpL strains, the Pto DC3000ΔhrpL strain was transformed with the plasmids NPTIIₚᵣₒ:WT-HrpL and NPTIIₚᵣₒ:mut-HrpL, respectively, by electroporation and then plated in NYGB medium with gentamycin.

### RNA Gel Blot Analyses

To perform northern blot analyses of low molecular weight RNAs, total RNA was extracted using TriZOL reagent and stabilized in 50% formamide. Around 30 μg of total RNA from the specified conditions were used to perform Northern blot analyses as previously described (49). Regions of 150 bp to 300 bp were amplified from the plasmids using gene specific primers and the amplicons were further used to generate specific 32P-radiolabelled probes synthesized by random priming. U6 probe was used as a control for equal loading of small RNAs.

### Separation of Long and Small RNA Fractions

Total RNAs were extracted from Arabidopsis leaves of IR-CFA6/HRPL #4 using Tri-Reagent (Sigma, St. Louis, Mo.) according to the manufacturer's instructions. Using 100 μg of total RNA, long and small RNA fractions were separated using the mirVana miRNA isolation kit (Ambion, Life technologies) according to the manufacturer's instructions. The separation of long and small RNAs from the total RNAs was visualized using agarose gel electrophoresis and further analyzed using microfluidic based approach (Bioanalyzer 2100; Agilent Technologies, http://www.agilent.com). The total, long and small RNAs were further used to perform the stomatal reopening assay.

### Bacterial Infection Assays in Plants

(a) Bacterial growth assay: Plants for this experiment were specifically used after three hours of beginning of the night cycle in growth chamber. Three plants per condition were dip-inoculated using the bacterium at 5×10⁷ cfu/ml with 0.02% Silwet L-77 (Lehle seeds). Plants upon bacterial dipping were immediately placed in chambers with high humidity to facilitate proper infection. Water-soaking symptoms upon dip-inoculation were observed 24 hours post-infection and pictures of leaves from three plants per condition were taken. Two days post-inoculation, bacterial titer for each mentioned condition was measured for individual infected leaf as described in (49). To quantify bacterial transcripts in infected plants, pool of infected leaf samples was collected three days post-inoculation.

(b) Wound-inoculation assay: To monitor the propagation of bacteria in the midveins, around 15 leaves from three plants per condition were manually inoculated with a toothpick dipped in GFP-tagged bacteria at a concentration of 5×10⁶ cfu/ml and then the plants were placed in chambers with high humidity for 3 days. Bacterial propagation was then analyzed by monitoring GFP signal under a UV light using an Olympus MV 10× macrozoom and pictures were taken with a CCD camera AxioCam Mrc Zeiss with a GFP filter.

(c) Plant protection assay: Prior to bacterial infection, four rosette leaves of three Arabidopsis plants per condition were individually treated by repeatedly soaking with mock solution or RNA solutions at a concentration of 20 ng/μl of specific total RNAs, both supplemented with Silwett L-77 (0.02%). One hour after pretreatment, leaves were dip-inoculated with Pto DC3000 WT or Pto DC3000Δcfa6 at a concentration of 5×10⁷ cfu/ml in similar way as that of RNAs. Bacterial titers were monitored two days post-inoculation, as specified earlier. In tomato, two leaves of three plants per condition were pretreated with a suspension having 20 ng/μl of specific total RNA supplemented with Silwett L-77 (0.02%) and then were dipped one hour after with GFP-tagged Pto DC3000 at 5×10⁷ cfu/ml. The plants were then placed in controlled conditions at 24° C./19° C. (day/night) with a 16 h photoperiod without lid cover for 3 days. Bacterial infection was then analyzed by monitoring GFP signal under a UV light using an Olympus MV 10× macrozoom and pictures were taken. Individual leaf samples were collected to quantify the amount of bacteria in each condition using ImageJ software.

### Bacterial Luminescence Quantification

Three plants per condition were syringe-infiltrated with Pto DC3000 Luciferase (Pto Luc) strain at 1×10⁶ cfu/ml. Plants were placed in chambers with high humidity to facilitate proper infection. Leaf discs were placed in individual wells of a 96 well plate to quantify the luminescence using Berthold Centro LB 960 Microplate Luminometer. Four leaves per plant were taken into consideration. Leaf discs from individual leaves were pooled after to perform bacterial titer quantification as mentioned above.

### Tomato Infection Quantification

(a) GFP loci quantification: Tomato leaves infected with Pto DC3000-GFP strain were subjected to GFP quantification under a UV light using an Olympus MV 10× macrozoom and pictures were taken with a CCD camera AxioCam Mrc Zeiss with a GFP filter. Number of GFP loci was quantified with ImageJ software for at least 10 pictures per condition.

### (b) Bacterial Genomic DNA Quantification

To quantify bacterial infection in the infected tomato plants (Ross et al., 2006), the amount of bacterial genomic DNA (gDNA) was measured relative to plant gDNA. Genomic DNA was isolated from tomato leaf samples infected with Pto DC3000-GFP using the DNeasy plant mini kit (QIAGEN, Germany) according to the manufacturer's instructions. Using 1 ng of gDNA, qPCR was performed using Takyon SYBR Green Supermix (Eurogentec®) and GFP gene-specific primers. Amount of bacterial gDNA was normalized to that of tomato using Ubiquitin-specific primers.

Agrobacterium-Mediated Transient Expression of Inverted Repeats in N. benthamiana

To produce single hairpins, IR-CFA6 and IR-HRPL, and the chimeric hairpin IR-CFA6/HRPL, the A. tumefaciens strain carrying the plasmids were grown overnight in LB medium at 28° C. Cells were harvested by centrifugation and resuspended in a solution containing 10 mM MES, pH 5.6, 10 mM MgCl₂ and 200 μM acetosyringone at a final density of 0.5 OD₆₀₀. Cultures were incubated in the dark at room temperature for 5-6 hours before Agrobacterium-mediated infiltration in four-week old N. benthamiana. After 3 days of infiltration, leaf tissue was harvested and Northern blot analysis was performed to confirm the production of anti-Cfa6 and anti-HrpL siRNAs. The leaf samples were then used for total RNA extraction.

### In Vitro Antibacterial Gene Silencing Assay

To assess whether the bacterial transcripts Cfa6 and HrpL can be directly targeted by the dsRNA and/or the siRNAs generated by the hairpin IR-CFA6/HRPL, 2 ml culture of Pto DC3000 WT, Pto DC3000-WT-HrpL and Pto DC3000-mut-HrpL at 10⁷ cfu/ml was treated for 4 and/or 8 hours, with 20 ng/μl of specified total RNA extracted from CV or IR-CFA6/HRPL #4 transgenic plants in a six-well plate, respectively. Similarly, to quantify the silencing of bacterial genes upon treatments with in vitro synthesized siRNAs, 2 ml of Pto DC3000-GFP at 1×10⁷ cfu/ml was treated for 6 hours with 2 ng/μl of in vitro synthesized IR-CYP51 siRNAs or IR-CFA6/HRPL siRNAs in a six-well plate, respectively. Bacteria were collected for each condition and further processed for molecular analyses.

### Apoplastic Fluid (AF) and Extracellular Vesicles (EVs) Extraction

Extraction was done as previously described (44). Sixty leaves of 5 week-old CV or IR-CFA6/HRPL plants were infiltrated with Vesicle Isolation Buffer (VIB; 20 mM MES, 2 mM 324 CaCl₂, 0.01 M NaCl, pH 6.0) with a syringe without needle. Leaves were then placed inside a 20 ml needless syringe. Syringe was then placed in 50 ml Falcon and centrifuged at 900 g for 15 minutes. The apoplastic fluid (APF) was collected and centrifuged subsequently at 2,000 g and 10,000 g for 30 minutes to get rid of any cell debris and then passed through a 0.45 μm filter. The APF was further subjected to ultracentrifugation step at 40,000 g to pellet EV fraction (P40). The pellet was resuspended in 2 ml of 2004 Tris buffer pH=7.5. The supernatant was then subjected to ultracentrifugation step at 100,000 g to pellet EV fraction (P100). The supernatant from this step was restored (SN).

### Stomatal Aperture Measurements

Plants were kept under light (100 μE/m²/s) for at least 3 hours before subjecting to any treatment to assure full expansion of stomata. Intact leaf sections from three four-week-old plants were dissected and immersed in water (Mock) or bacterial suspension at a concentration of 1×10⁸ cfu/ml. After 3 hours of treatment, unpeeled leaf abaxial surface was observed under SP5 laser scanning confocal microscope and the pictures were taken from different regions. The stomatal aperture (width/length) was measured using ImageJ software for 30-70 stomata per condition. In case of RNA pretreatments, the leaf sections were incubated with total RNAs extracted from specified genotypes for one hour before incubation with the bacteria. When required in specified experiments, 1 μM of exogenous Coronatine (COR) (Sigma) (50) was supplemented to the bacterial suspension.

### Real-Time RT-PCR Analyses

To monitor plant-encoded transcripts, total RNA was extracted from plant samples using RNeasy Plant Mini kit (Qiagen). 0.5 μg of DNA-free RNA was reverse transcribed using qScript cDNA Supermix (Quanta Biosciences). cDNA was then amplified by real time PCR reactions using Takyon SYBR Green Supermix (Eurogentec®) and transcript-specific primers. Expression was normalized to that of Ubiquitin. To monitor bacterial transcripts, total RNA was extracted from bacteria-infected plant samples or from in vitro treated bacteria as described previously. After DNAse treatment, 250 ng of total RNA was reverse transcribed using random hexamer primers and qScript Flex cDNA kit (Quanta Biosciences). cDNA was then amplified by real time PCR reactions using Takyon SYBR Green Supermix (Eurogentec®) and transcript-specific primers. Expression was normalized to that of GyrA. PCR was performed in 384-well optical reaction plates heated at 95° C. for 10 min, followed by 45 cycles of denaturation at 95° C. for 15 s, annealing at 60° C. for 20 s, and elongation at 72° C. for 40 s. A melting curve was performed at the end of the amplification by steps of 1° C. (from 95° C. to 50° C.).

### In Vitro Synthesis of Inverted Repeat (IR) RNAs

In vitro synthesis of RNAs was generated following the instruction of the MEGAscript® RNAi Kit (Life Technologies, Carlsbad, Calif.). Templates like were amplified by PCR introducing the T7 promotor at both 5′ and 3′ end of the sequence. PCR amplification was done in two steps with two different annealing temperature to rise the specificity of primers annealing. After the amplification step, PCR products were purified by gel extraction thanks to the NucleoSpin® Gel and PCR Clean-up kit (Macherey-Nagel) to eliminate any parasite amplification. Those purified PCR products were then used as templates for in vitro transcription: 2 μg was incubated for five hours at 37° C. with 2 μL of T7 polymerase (T7 enzyme Mix), 2 μL of 10×T7 Reaction Buffer and 2 μL of each 75 mM ATP, CTP, GTP and UTP. The total volume is adjusted to 20 μL with Nuclease free water. After the transcription reaction, dsRNAs were treated with 2 μL of DNaseI, 2 μL of RNase, 5 μL, of 10× reaction buffer to eliminate DNA templates and single stranded RNAs. Then, dsRNAs are purified with the filter cartridges provided with the kit. Long dsRNA obtained at this step are used for the following experiments (FIG. 1). siRNAs were obtained thanks to ShortCut® RNase III (NEB, Ipswich, Mass.). DsRNAs were digested for 20 minutes with RNaseIII and then purified thanks to the mirVana™ miRNA Isolation Kit (Life Technologies, Carlsbad, Calif.). After purification, siRNAs are used for the following experiments (FIG. 1). Each steps of the process were followed by gel electrophoresis (TAE 1×, 1% agarose gel for DNA amplification and 2% agarose gel for RNAs) to check the quality of RNAs.

### Droplet-Based Microfluidic Assay for the Monitoring of In Vitro Pto DC3000 Growth

Droplet-based microfluidic experiments were performed in NYGB medium at a temperature of 28° C. RNAi assays were prepared by pipetting directly in the 96 well plate the different solutions to obtain 200 μl final: 100 μl of medium, 20 μl of a GFP-tagged Pto DC3000 (Pto DC3000-GFP) at 10⁷ cfu/ml, 20 μl of in vitro synthesized candidate siRNAs to obtain a final concentration at 2 ng/μl or sterile water for the control sample followed by 60 μl of medium. In case of mix of different RNAs, a v/v ratio of the different RNAs was prepared and 20 μl of the mix was added to the corresponding wells. The 96-well plate was set on the machine for the samples to be fractioned in droplets by the Millidrop Analyzer (http://www.millidrop.com). For each well, 10 droplets of ˜500n1 each were formed and incubated inside the instrument for the 24 hours. For each droplet, measurements of biomass and GFP fluorescence were acquired every ˜30 minutes.

### Example 2. Arabidopsis-Encoded siRNAs Directed Against Either Endogenous Virulence Factors or Artificial Reporter Genes from Pto DC3000 Trigger their Silencing in the Context of Bacterial Infection

To test whether host-encoded small RNAs could alter bacterial gene expression, we have generated Arabidopsis stable transgenic plants that constitutively express a chimeric inverted repeat bearing sequence homology to the ECF-family sigma factor HrpL gene and the coronatine (COR) biosynthesis, Cfa6 gene, both of which encode key virulent determinants of Pto DC3000 (FIG. 1A, (51, 52)). As negative controls, we have also generated transgenic lines overexpressing an inverted repeat bearing sequence homology to three cytochrome P450 lanosterol C-14α-demethylase (CYP51) genes of the fungus F. graminearum, which was previously shown to confer full protection against this fungal phytopathogen in both Arabidopsis and barley (20,21). These stable transgenic lines are referred to as IR-CFA6/HRPL and IR-CYP51 (or CV, Control Vector plants), respectively; and do not exhibit any developmental defect (FIG. 1B), despite high accumulation of artificial siRNAs (FIG. 1C). To investigate whether artificial siRNAs directed against Cfa6 and HrpL could interfere with the expression of these virulence factors during bacterial infection, we dip-inoculated the above transgenic plants with Pto DC3000 and further monitored Cfa6 and HrpL mRNA levels by RT-qPCR analyses. While the Cfa6 mRNA levels were moderately altered in two out of three independent IR-CFA6/HRPL lines compared to Col-0 plants, the levels of HrpL transcripts were reproducibly reduced in all the three IR-CFA6/HRPL lines compared to Col-0 plants at this timepoint (FIG. 1D). By contrast, the down-regulation of Cfa6 or HrpL mRNAs was not observed in IR-CYP51-versus Col-0-infected plants (FIG. 1D), supporting a specific effect of these antibacterial RNAs in this regulatory process. Similarly, the mRNA level of the non-targeted ProC gene was unchanged in both IR-CFA6/HRPL- and IR-CYP51-infected lines compared to Col-0-infected plants (FIG. 1D). Collectively, these data indicate that the Arabidopsis-encoded IR-CFA6/HRPL inverted repeat can at least trigger sequence-specific silencing of the bacterial HrpL transcript in the context of infection.

Because the expression of HrpL and Cfa6 virulence factors is known to be regulated by various environmental cues (52, 53), we also tested whether AGS could be effective against the Photorhabdus luminescens luxCDABE operon chromosomally expressed in Pto DC3000 under the constitutive kanamycin promoter (54). This lux-tagged Pto DC3000 strain spontaneously emits luminescence because it co-expresses the luciferase catalytic components luxA and luxB genes along with the genes required for substrate production, namely luxC, luxD and luxE (55). Two independent Arabidopsis transgenic lines, IR-LuxA/LuxB lines, overexpressing anti-luxA and anti-luxB siRNAs were selected and syringe-infiltrated with the lux-tagged Pto DC3000 strain (FIG. 2A/B). The levels of luxA and luxB mRNAs as well as the luminescence activity were further monitored at 24 hours post-inoculation (hpi). By doing so, we found a significant reduction in both luxA and luxB mRNA abundance as well as in luminescence activity in IR-LuxA/LuxB-compared to Col-0-infected plants (FIG. 2C). By contrast, the growth of the bacterial reporter strain was unchanged in IR-LuxA/LuxB lines compared to Col-0 plants in those conditions (FIG. 2D), indicating that the above effects were not due to a decreased bacterial titer in these transgenic plants. Altogether, these data indicate that AGS is effective against both endogenous stress-responsive bacterial genes and exogenous constitutive bacterial reporter genes during Pto DC3000 infection.

### Example 3. Host-Encoded siRNAs Directed Against Cfa6 and HrpL Prevent Pto DC3000-Induced Stomatal Reopening Presumably by Suppressing Coronatine Biosynthesis

Because Cfa6 and HrpL are known to regulate each other (53) and because HrpL and Cfa6 are both essential for coronatine (COR) biosynthesis (52, 53), we next investigated whether IR-CFA6/HRPL plants could be protected from COR-dependent virulence responses. For this purpose, we monitored Pto DC3000-triggered stomatal reopening at 3 hours post-inoculation (3 hpi), a phenotype that is fully dependent on COR biosynthesis and thus abolished upon inoculation with Pto DC3000 mutants that are either deleted in Cfa6 or HrpL genes (FIG. 3A, (50)). It is noteworthy that this phenotype is not dependent on type III effectors at this timepoint of infection because a normal stomatal reopening response was observed upon treatment with the Pto DC3000 hrcC mutant (FIG. 3A, (50)), which is impaired in the assembly of the type III secretion system. Significantly, we found that Pto DC3000-induced stomatal reopening was fully abolished in the three independent IR-CFA6/HRPL transgenic lines infected with the virulent Pto DC3000 strain as compared to Col-0-infected leaves (FIG. 3B), thereby mimicking the phenotype observed on Col-0 leaves inoculated with the Pto DC3000 cfa6- or hrpl-deleted strains (FIG. 3A). By contrast, a normal Pto DC3000-induced stomatal reopening was observed in IR-CYP51-infected plants (FIG. 3C), indicating that the observed effect is specific to siRNAs directed against Cfa6 and HrpL genes. Furthermore, the compromised stomatal reopening phenotype detected in IR-CFA6/HRPL-infected transgenic plants was fully rescued upon exogenous application of COR (FIG. 3B). These data provide thus pharmacological evidence that the reduced Pto DC3000 pathogenesis manifested at infected IR-CFA6/HRPL stomata is likely caused by an altered ability of the associated and/or surrounding bacterial cells to produce COR.

### Example 4. Arabidopsis Stable Transgenic Plants Expressing Small RNAs Against Key Virulence Factors from Pto DC3000 or Xanthomonas campestris pv. Campestris are Protected from Bacterial Infections

To further monitor the possible effects that anti-Cfa6 and anti-HrpL siRNAs could have on Pto DC3000 pathogenicity, we next monitored the ability of this bacterium to spread in the leaf vasculature of Arabidopsis IR-CFA6/HRPL transgenic plants. For this purpose, we scored the number of bacterial spreads occurring at three sites from the midvein of individual leaves wound-inoculated with a virulent GFP-tagged Pto DC3000 (Pto DC3000-GFP) strain. Using this quantification method, we observed an index of bacterial propagation that was significantly decreased in the three independent IR-CFA6/HRPL transgenic lines as compared to Col-0 plants (FIG. 4A). This suggests that siRNAs directed against Cfa6 and HrpL can reach xylem vessels and further dampen the virulence activity of Pto DC3000 in Arabidopsis leaf vasculature. By contrast, a normal Pto DC3000 vascular spreading was observed in the IR-CYP51 transgenic line compared to Col-0-infected leaves (FIG. 4A), arguing for a specific effect of anti-Cfa6 and anti-HrpL siRNAs in this process. Collectively, these results indicate that siRNAs directed against the pathogenicity determinants Cfa6 and HrpL can specifically restrict the spreading of Pto DC3000 in Arabidopsis leaf vasculature. An enhanced vascular disease protection effect towards the Gram-negative bacterium Xanthomonas campestris pv. campestris (Xcc) was also found in Arabidopsis transgenic plants overexpressing siRNAs against the virulence factors HrpX, HrpG and RsmA (FIG. 5, data not shown, (56-60)). This demonstrates that AGS can additionally be used to protect plants against this well-characterized vascular bacterial pathogen of Arabidopsis, which is the causal agent of black rot, one of the most devastating diseases of crucifer crops worldwide (25, 61).

We next investigated whether stable expression of siRNAs against Cfa6 and HrpL could also impact growth of Pto DC3000 in planta, a phenotype known to be dependent on both COR and on a functional type III secretion system (52). To this end, we dip-inoculated IR-CFA6/HRPL, IR-CYP51 and WT plants with Pto DC3000 and further monitored bacterial titer at 48 hpi. Using this assay, we found a significant reduction in Pto DC3000 titer in the three independent IR-CFA6/HRPL transgenic lines compared to Col-0-infected plants, and this phenotype was reminiscent to the one observed in WT plants infected with a Cfa6-deleted strain (FIG. 4C). Interestingly, we additionally observed a reduced Pto DC3000-induced water soaking disease symptoms in the three independent IR-CFA6/HRPL plants compared to WT-infected plants at 24 hpi, which resemble the phenotype observed in WT leaves dip-inoculated with the Cfa6 mutant strain (FIG. 4D). By contrast, the bacterial growth and water soaking disease symptoms were unaltered in IR-CYP51 transgenic plants dip-inoculated with Pto DC3000 (FIG. 4C/D), indicating that the above effects are specific to siRNAs directed against Cfa6 and HrpL genes. Altogether, these data further support a major role for anti-Cfa6 and anti-HrpL siRNAs in dampening the virulence activity of Pto DC3000 in the context of infection. They also provide compelling evidence that AGS is an effective strategy that can be used to control bacterial pathogenicity in stable transgenic plants.

### Example 5. Exogenous Delivery of Total RNAs Derived from IR-CFA6/HRPL Plants Protect WT Arabidopsis and Tomato Plants Against Pto DC3000

Environmental RNAi is a phenomenon by which (micro)organisms can uptake external RNAs from the environment, resulting in the silencing of genes containing sequence homologies to the RNA triggers (24). This RNA-based process has been initially characterized in C. elegans (26-29), and was further found to operate in other nematodes but also in insects, plants and fungi (26, 30). However, this approach has never been used against a bacterial phytopathogen that lacks a canonical eukaryotic-like RNAi machinery such as Pto DC3000. To test this possibility, we first assessed whether RNAs expressed from IR-CFA6/HRPL plants could trigger silencing of Cfa6 and HrpL genes in in vitro conditions. For this purpose, we extracted total RNAs from CV and IR-CFA6/HRPL plants, incubated them with Pto DC3000 cells, and further analyzed by RT-qPCR the levels of Cfa6 and HrpL mRNAs at 4 and 8 hours after RNA treatments. Results from these analyses revealed a reduced accumulation of both virulence factor mRNAs upon treatment with RNA extracts from IR-CFA6/HRPL plants, a molecular effect that was not observed with RNA extracts derived from CV plants (FIG. 6A). By contrast, the level of the non-targeted ProC and RpoB mRNAs remained unaltered in the same conditions (FIG. 6A). These data therefore imply that plant antibacterial RNAs are likely taken-up by Pto DC3000 cells and subsequently trigger sequence-specific silencing of Cfa6 and HrpL genes. It also suggests that exogenous application of these antibacterial RNAs could be used as a strategy to dampen Pto DC3000 pathogenesis in Col-0 plants. To test this intriguing hypothesis, we pre-treated Arabidopsis Col-0 leaf tissues with total RNA extracts from IR-CFA6/HRPL plants for one hour, subsequently challenged them with Pto DC3000 for 3 hours, and further monitored bacterial-induced stomatal reopening events. Strikingly, we found that RNA extracts from IR-CFA6/HRPL plants fully suppressed the ability of Pto DC3000 to reopen stomata (FIG. 6B), thereby mimicking the phenotype observed in infected IR-CFA6/HRPL transgenic plants (FIG. 3). We additionally investigated whether this approach could be used to control the growth of Pto DC3000 in planta. For this purpose, we first pre-treated for one hour Col-0 Arabidopsis plants with total RNA extracts from IR-CFA6/HRPL plants and further dip-inoculated them with Pto DC3000. We found that these RNA extracts triggered a decreased Pto DC3000 titer at 2 dpi (FIG. 6C), a phenotype that was comparable to the ones observed in infected IR-CFA6/HRPL transgenic plants (FIG. 4C), as well as in Col-0 plants inoculated with the PtoΔcfa6 strain (FIG. 6C). By contrast, application of total RNA extracts from CV plants did not alter growth of Pto DC3000 in the same conditions (FIG. 6C), supporting a specific effect of antibacterial RNAs in this process. To assess whether such RNA-based biocontrol approach could also be effective in cultivated plants, we repeated the same assay on tomato (Solanum lycopersicum, cultivar Moneymaker), which is the natural host of Pto DC3000. Pre-treatment of WT tomato leaves for one hour with RNA extracts from IR-CFA6/HRPL plants led to compromised Pto DC3000-induced necrotic disease symptoms and also to a reduction in bacterial content compared to leaves pre-treated with RNA extracts derived from CV plants (FIG. 6D-F). Collectively, these data provide evidence that external application of plant-derived antibacterial RNAs can trigger AGS and disease protection against Pto DC3000 in both Arabidopsis and tomato plants.

### Example 6. Small RNA Species, but not their dsRNA Precursors, are Causal for the Compromised Stomatal Reopening Phenotype Observed Upon Exogenous Application of Total RNAs Derived from the IR-CFA6/HRPL Hairpin

Next, we interrogated which RNA entities are responsible for AGS and pathogenesis reduction upon external application of antibacterial RNAs. To address this question, we first crossed the IR-CFA6/HRPL #4 reference line with the dcl2-1 dcl3-1 dcl4-2 (dcl234) triple mutant and subsequently selected F3 plants that were homozygous for the three dcl mutations and for the IR-CFA6/HRPL transgene. Molecular characterization of these IR-CFA6/HRPL #4×dcl234 plants revealed an enhanced accumulation of IR-CFA6/HRPL inverted repeat transcripts (i.e. unprocessed dsRNAs) compared to the level detected in IR-CFA6/HRPL #4 parental line (FIG. 7A). Furthermore, this effect was associated with undetectable levels of anti-Cfa6 and anti-HrpL siRNAs (FIG. 7A). These data are thus consistent with a role of DCL2, DCL3 and DCL4 in the biogenesis of these siRNAs through the processing of the IR-CFA6/HRPL inverted repeat. We subsequently extracted total RNAs from these plants, incubated them with Pto DC3000 cells for 8 hours, and further monitored Cfa6 and HrpL mRNA levels by RT-qPCR analysis. Using this in vitro assay, we found that RNA extracts from IR-CFA6/HRPL #4×dcl234 plants were no longer able to trigger down-regulation of Cfa6 and HrpL mRNAs (FIG. 7B), despite high accumulation of artificial dsRNA precursors (FIG. 7A). By contrast, RNA extracts from the IR-CFA6/HRPL #4 parental line, which contain high levels of anti-Cfa6 and anti-HrpL siRNAs (FIG. 7A), triggered reduced accumulation of both targeted virulence factors (FIG. 7B). Moreover, while RNA extracts from IR-CFA6/HRPL #4 plants suppressed Pto DC3000-induced stomatal reopening events, we found that RNA extracts from IR-CFA6/HRPL #4×dcl234 plants were inactive in this process, such as control RNA extracts derived from Col-0 or dcl234 plants (FIG. 7C, data not shown). Collectively, these data provide compelling evidence that dsRNAs produced from the IR-CFA6/HRPL inverted repeat are neither involved in AGS nor in pathogenesis reduction. They rather suggested that small RNAs are likely the antibacterial RNA entities responsible for these molecular and physiological phenotypes. To verify this assumption, we further purified small RNA species from IR-CFA6/HRPL plant total RNAs using a glass fiber filter-based method (FIG. 7D), and subjected them to stomatal reopening assay. By doing so, we found that these small RNA species suppressed Pto DC3000-triggered stomatal reopening, to the same extent as IR-CFA6/HRPL plant total RNA extracts (FIG. 7E). By contrast, long RNA species (above 200 bp), which were not filtered through the above columns, were inactive (FIG. 7E), further supporting that antibacterial plant dsRNAs are not involved in this response. Altogether, these data provide solid evidence that DCL-dependent siRNAs produced from the inverted repeat IR-CFA6/HRPL are critical for AGS and pathogenesis reduction, while cognate dsRNA precursors are ineffective for both processes.

### Example 7. A Bacterially Expressed Small RNA Resilient Version of HrpL is Insensitive to siRNA-Directed Silencing and Exhibits a Normal Stomatal Reopening Phenotype Indicating that Anti-HrpL siRNAs are Causal for AGS and Pathogenesis Reduction

Although the above findings indicate that external application of antibacterial siRNAs can trigger AGS and antibacterial activity, they do not firmly demonstrate that these RNA entities are causal for these phenomena. To address this issue, we decided to generate and characterize recombinant bacteria expressing a siRNA-resilient version of the HrpL gene, which was found to be subjected to AGS regulation in both in vitro and in planta conditions (FIGS. 1 and 6). To this end, we complemented the PtoΔhrpL mutant with either a WT HrpL transgene or a mutated version, mut HrpL that contains as many silent mutations as possible in the siRNA targeted region, which are predicted to alter the binding of siRNAs with the HrpL mRNA but to produce the same protein sequence. Furthermore, to assess the post-transcriptional regulatory control that anti-HrpL siRNAs might exert over these bacterial transgenes, we expressed them under the constitutive neomycin phosphotransferase II (NPTII) promoter. The two resulting recombinant bacteria are referred to as PtoΔhrpL WT HrpL and PtoΔhrpL mut HrpL, respectively, and were found to restored ability to reopen stomata when inoculated on Col-0 plants (FIG. 8A, data not shown), indicating that both transgenes are functional. We further assessed the sensitivity of each recombinant bacterium to AGS. For this purpose, we incubated PtoΔhrpL WT HrpL and PtoΔhrpL mut HrpL strains with total RNA extracts from CV and IR-CFA6/HRPL #4 plants for 8 hours and further monitored HrpL transgene mRNA levels by RT-qPCR analysis. We found a significant decrease in the accumulation of HrpL mRNAs expressed from the PtoΔhrpL WT HrpL strain, which was not detected upon treatment with control RNA extracts from CV plants (FIG. 8B). These data indicate that the WT HrpL transgene expressed from the PtoΔhrpL WT HrpL strain is fully sensitive to AGS despite its constitutive expression driven by the NPTII promoter. By contrast, the accumulation of HrpL mRNAs expressed from the PtoΔhrpL mut HrpL strain was unaltered in response to RNA extracts from IR-CFA6/HRPL #4 plants (FIG. 8B), indicating that siRNAs no longer exert their AGS effect towards this recombinant bacterium. Collectively, these findings demonstrate that anti-HrpL siRNAs are causal for the post-transcriptional silencing of the HrpL virulence factor gene within Pto DC3000 cells. Next, we investigated the responsiveness of each recombinant bacterial strain to siRNA-directed pathogenesis reduction by exploiting the Pto DC3000-induced stomatal reopening assay, which is highly sensitive to small RNA action. To assess the specific effect of siRNAs towards suppression of HrpL-mediated stomatal reopening function, we first cloned an IR-HRPL inverted repeat targeting the same HrpL sequence region than the one targeted by the IR-CFA6/HRPL hairpin, and further validated its capacity to produce HrpL siRNAs upon Agrobacterium-mediated transient transformation in Nicotiana benthamiana leaves (FIG. 8C). N. benthamiana total RNA extracts containing anti-HrpL siRNAs were found to fully suppress the ability of Pto DC3000 to reopen stomata (FIG. 8D). Importantly, similar results were obtained when N. benthamiana RNA extracts containing anti-HrpL siRNAs were incubated with the PtoΔhrpL WT HrpL strain (FIG. 8D), supporting a sensitivity of this bacterial strain to siRNA action. By contrast, the PtoΔhrpL mut HrpL strain was fully competent in reopening stomata in the same conditions (FIG. 8D), indicating that anti-HrpL siRNAs no longer exert their antibacterial effects towards this recombinant bacterial strain. These data provide thus evidence that anti-HrpL siRNAs are causal for the suppression of HrpL-mediated stomatal reopening function. They also further validate a novel role of HrpL in bacterial-induced stomatal reopening, indicating that AGS can be employed as a tool to characterize bacterial gene function.

### Example 8. The Apoplastic Fluid of IR-CFA6/HRPL Plants is Composed of Functional Antibacterial siRNAs that are Either Embedded into EVs, and Protected from Micrococcal Nuclease Action, or in a Free Form, and Sensitive to Micrococcal Nuclease Digestion

The results from the phenotypical analyses described in EXAMPLES 3 and 4 imply that small RNA species that are constitutively expressed in IR-CFA6/HRPL transgenic lines, must be externalized from plant cells towards the leaf surface, the apoplastic environment and xylem vessels in order to reach epiphytic and endophytic bacterial populations. To get some insights into the small RNA trafficking mechanisms that could be implicated in this phenomenon, we have first extracted the apoplastic fluid (APF) from IR-CFA6/HRPL plants and tested its ability to dampen bacterial pathogenesis by monitoring its impact on Pto DC3000-induced stomatal reopening. We found that this extracellular fluid triggered a full suppression of stomatal reopening during infection, thereby mimicking the effect triggered by IR-CFA6/HRPL-derived total RNAs (FIG. 9A). By contrast, the APF from IR-CYP51 plants was inactive, supporting a specific effect of anti-Cfa6 and anti-HrpL siRNAs from the AFP of IR-CFA6/HRPL plants in this process (FIG. 9A). We further tested whether EVs from IR-CFA6/HRPL plants could contribute to AGS. For this end, we recovered APF from IR-CFA6/HRPL plants and further performed differential ultracentrifugation at 40,000 g or 40,000 g followed by 100,000 g, which allowed us to collect two fractions, named P40 and P100, respectively. Interestingly, we found that both fractions were capable of suppressing stomatal reopening, although P100 was moderately less effective in this process (FIG. 9B). Importantly, both fractions remained active in the presence of micrococcal nuclease (Mnase), indicating that small RNAs are protected from external degradation when embedded into EVs. Intriguingly, we also noticed that the supernatant fraction (SN), recovered after the sequential centrifugation at 40,000 g and 100,000 g, exhibited strong antibacterial activity, despite a lack of canonical EVs detected in this fraction (FIG. 9B, data not shown). This suggests that EV-free small RNAs that are either associated with proteins and/or in a free-form could additionally be competent for AGS. To determine which of the two small RNA entities could possess such antibacterial activity, we treated SN fractions from IR-CFA6/HRPL plants with Mnase or proteinase K and further subjected them to stomatal reopening assay. Interestingly, we found that the Mnase treatment abrogated the antibacterial effect triggered by the IR-CFA6/HRPL-derived SN fraction, while an unaltered antibacterial activity was detected in the presence of proteinase K, which globally degraded proteins (FIG. 9B, data not shown). Collectively, these data indicate that functional EV-free antibacterial small RNAs are unlikely associated with proteins and are thus referred to here as Extracellular Free Small RNAs or “efsRNAs”. Our results also indicate that efsRNAs are sensitive to Mnase action because they lost their antibacterial effect upon treatment with this nuclease (FIG. 9B). Based on these findings, we propose that the APF from IR-CFA6/HRPL plants is composed of at least three populations of functional antibacterial small RNAs, which are 1) embedded into large EVs (P40 fraction), 2) embedded into EVs of smaller size (P100 fraction), or 3) in a free form.

### Example 9. The In Vitro Synthesis of Small RNAs is an Easy, Rapid and Reliable Approach to Screen for Candidate Small RNAs Possessing Antibacterial Activities

In order to develop a screening platform for the identification of candidate small RNAs with antibacterial activities, we aimed to produce in vitro synthesized siRNAs against specific bacterial gene transcripts and further test their activities on bacterial pathogenicity or survival. For this end, we first decided to generate in vitro synthesized anti-Cfa6 and anti-HrpL siRNAs targeting the same sequences than the plant siRNAs produced from the DCL-dependent processing of IR-CFA6/HRPL. To do so, we used primers carrying T7 promoter sequences to amplify either CYP51 or CFA6/HRPL DNA from plasmids containing the IR-CYP51 or IR-CFA6/HRPL sequences. The resulting PCR products were gel-purified and subsequently used as templates for in vitro RNA transcription using a T7 RNA polymerase, which led to the production of CYP51 or CFA6/HRPL dsRNAs of expected size (FIG. 10A). Small RNAs were further obtained by digesting these dsRNAs into 18-25 bp siRNAs using the ShortCut® RNase III, although other non-commercial RNase III can also be used for this process (data not shown). As revealed by agarose gel electrophoresis, these siRNAs were deprived of dsRNA (FIG. 10A), indicating that the RNase III used in these experiments fully processed the initial pool of dsRNA molecules. We next analyzed the ability of synthetic dsRNA and siRNAs to suppress stomatal reopening. Consistent with our previous data showing that plant dsRNAs are inactive in triggering AGS (FIG. 7), we found that in vitro synthesized CFA6/HRPL dsRNAs did not interfere with Pto DC3000-induced stomatal reopening, nor did in vitro synthesized CYP51 dsRNAs, which were used as negative controls (FIG. 10B). By contrast, in vitro synthesized siRNAs directed against Cfa6 and HrpL fully prevented Pto DC3000-induced stomatal reopening, while in vitro synthesized anti-CYP51 siRNAs were inactive in this process (FIG. 10B). The latter result suggested that in vitro synthesized anti-Cfa6 and anti-HrpL siRNAs were likely capable of triggering silencing of Cfa6 and HrpL genes. To test this hypothesis, we further incubated the in vitro synthesized CYP51 and CFA6/HRPL siRNAs at a concentration of 2 ng/ul with 1×10⁷ cfu/ml of Pto DC3000 for 6 hours and further monitored Cfa6 and HrpL mRNAs by RT-qPCR analyses. By doing so, we found that anti-Cfa6/HrpL siRNAs triggered a significant reduced accumulation of Cfa6 and HrpL mRNAs compared to anti-CYP51 siRNAs (FIG. 10B), a molecular effect which was comparable to the one observed in response to plant-derived total RNAs containing anti-Cfa6 and anti-HrpL siRNAs (FIG. 6A, 7B). By contrast, the levels of the non-targeted ProC and RpoB mRNAs remained unchanged in response to anti-Cfa6 and anti-HrpL siRNAs compared to anti-CYP51 siRNAs (FIG. 10B). Collectively, these data indicate that in vitro synthesized siRNAs can trigger AGS and antibacterial activity to the same extent as plant-derived anti-Cfa6 and anti-HrpL siRNAs.

We next decided to determine whether this approach could be instrumental for the identification of candidate siRNAs with bactericidal activities. To test this idea, we performed in vitro synthesis of siRNAs directed against three conserved and housekeeping genes from Pto DC3000, namely SecE (PSPTO_0613, preprotein translocase SecE subunit), FusA (PSPTO_0623, translation elongation factor G) and GyrB (PSPTO_0004, DNA gyrase subunit B) and further monitor their impact on the in vitro growth of this bacterium. To do so, we took advantage of an established droplet-based microfluidic system, which is suitable for the accurate measurements of bacterial biomass and bacterially-expressed fluorescence reporter activity. By using this approach, we found that 0.33 ng/μl of in vitro synthesized siRNAs directed against FusA was capable of reducing both the biomass and the GFP signal from a GFP-tagged Pto DC3000 (Pto DC3000-GFP), compared to the conditions in the absence of siRNAs or in the presence of anti-SecE siRNAs (FIG. 10E, F). Strikingly, we did not detect any GFP signal nor bacterial biomass when the Pto DC3000-GFP strain was incubated with 1 ng/ul of in vitro synthesized anti-FusA siRNAs, nor when in vitro synthesized siRNAs directed against GyrB were applied at concentrations of either 0.33 ng/μ1 or 1 ng/μ1 (FIG. 10E, F). These data indicate that siRNAs directed against FusA and GyrB possess a potent bactericidal activity that mimics the effect that would be detected in the presence of an antibiotic. Based on these proof-of-concept experiments, we conclude that the in vitro synthesis of siRNAs is an easy, rapid and reliable approach to screen for novel candidate small RNAs with antibacterial activities. They also unveil a role for FusA and GyrB in the survival of Pto DC3000, which has not previously been reported for this bacterium. These results therefore further support the fact that AGS can be employed as a tool to characterize bacterial gene function.

### REFERENCES

1. Couto, D., Zipfel, C. (2016). Regulation of pattern recognition receptor signaling in plants. Nat Rev Immunol. 16, 537-552.
2. Jones, J D., and Dangl, J L. (2006). The plant immune system. Nature. 444, 323-329.
3. Block, A., Alfano, J R. (2011). Plant targets for Pseudomonas syringae type III effectors: virulence targets or guarded decoys? Curr Opin Microbiol. 14, 39-46.
4. Xin X F., Kvitko B., He S Y. (2018). Pseudomonas syringae: what it takes to be a pathogen. Nat Rev Microbiol. 16:316-328.
5. Jones, J D., Vance, R E., Dangl, J L. (2016). Intracellular innate immune surveillance devices in plants and animals. Science. 354, DOI: 10.1126/science.aaf6395.
6. Navarro, L., Zipfel, C., Rowland, O., Keller, I., Robatzek, S., Boller, T., Jones, J D. (2004). The transcriptional innate immune response to flg22. Interplay and overlap with Avr gene-dependent defense responses and bacterial pathogenesis. Plant Physiol. 135, 1113-28.
7. Tsuda, K and Katagiri, F. (2010). Comparing signaling mechanisms engaged in pattern-triggered and effector-triggered immunity. Curr Opin Plant Biol. 13: 459-65.
8. Hamilton A. J., Baulcombe, D C. A species of small antisense RNA in posttranscriptional gene silencing in plants. Science. 1999; 286(5441):950-2.
9. Staiger, D., Korneli, C., Lummer, M., and Navarro, L. (2013). Emerging role for RNA-based regulation in plant immunity. New Phytol. 197, 394-404.
10. Melnyk, C W., Molnar, A., Baulcombe, D C. (2011). Intercellular and systemic movement of RNA silencing signals. EMBO J. 30, 3553-63.
11. Baulcombe D C. (2015). VIGS, HIGS and FIGS: small RNA silencing in the interactions of viruses or filamentous organisms with their plant hosts. Curr Opin Plant Biol. 26, 141-6.
12. Weiberg, A., Bellinger, M., Jin H. (2015). Conversations between kingdoms: small RNAs. Curr Opin Biotechnol. 32, 207-15.
13. Koch, A., Kogel, K H. (2014). New wind in the sails: improving the agronomic value of crop plants through RNAi-mediated gene silencing. Plant Biotechnol J. 12, 821-31.
14. Zhang, T., Zhao, Y L., Zhao, J H., Wang, S., Jin, Y., Chen, Z Q., Fang, Y Y., Hua, C L., Ding, S W., Guo, H S. (2016). Cotton plants export microRNAs to inhibit virulence gene expression in a fungal pathogen. Nature Plants. DOI: 10.1038/nplants.2016.153.
15. Wang, M., Weiberg, Arne., Lin F M., Thomma, B P H., Huang, H D., Jin, H. (2016). Bidirectional cross-kingdom RNAi and fungal uptake of external RNAs confer plant protection. Nature plants. 16151.
16. Weiberg, A., Wang, M., Lin, F M., Zhao, H., Zhang, Z., Kaloshian, I., Huang, H D., Jin, H. (2013). Fungal small RNAs suppress plant immunity by hijacking host RNA interference pathways. Science. 342, 118-23.
17. Cai, Q., Qiao, L., Wang, M., He, B., Lin, F M., Palmquist, J., Huang, S D., Jin, H. (2018). Plants send small RNAs in extracellular vesicles to fungal pathogen to silence virulence genes. Science. 360, 1126-1129.
18. Micali, C O., Neumann, U., Grunewald, D., Panstruga, R., O'Connell. (2011). Biogenesis of a specialized plant-fungal interface during host cell internalization of Golovinomyces orontii haustoria. Cell Microbiol. 13, 210-226.
19. Hou, Y., Zhai, Y., Feng, L., Karimi, H., Rutter, B., Zeng, L., Seok Choi, D., Zhang, B., Gu, W., Chen, X., Ye, W., Innes R. W., Zhai, J., Ma, W. (2019). A phytophthora effector suppresses trans-kingdom RNAi to promote disease susceptibility. Cell Host & Microbe. 25, 153-165.
20. Koch A., Kumar N., Weber L., Keller, H., Imani J, Kogel, K H. (2013) Host-induced gene silencing of cytochrome P450 lanosterol C14 α-demethylase-encoding genes confers strong resistance to Fusarium species. Proc Natl Acad Sci USA. 110: 19324-9.
21. Koch, A., Biedenkopf, D., Furch, A., Weber, L., Rossbach, O., Abdellatef, E., Linicus, L., Johannsmeier, J., Jelonek, L., Goesmann, A., Cardoza, V., McMillan, J., Mentzel, T., Kogel, K H. (2016). An RNAi-based control of Fusarium graminearum infections through spraying of long dsRNAs involves a plant passage and is controlled by the fungal silencing machinery. PLoS Pathog. 12: e1005901.
22. Ghag S. B., Host induced gene silencing, an emerging science to engineer crop resistance against harmful plant pathogens. (2017). Physiological and Molecular Plant Pathology. 100: 242-254.
23. Lacombe, S., Rougon-Cardoso, A., Sherwood, E., Peeters, N., Dahlbeck, D., van Esse, H P., Smoker, M., Rallapalli, G., Thomma, B P., Staskawicz, B., Jones, J D., Zipfel, C. (2010). Interfamily transfer of a plant pattern-recognition receptor confers broad-spectrum bacterial resistance. Nat. Biotechnol. 28:365-9.
24. Wang, M., Thomas, N., Jin, H. (2017). Cross-kingdom RNA trafficking and environmental RNAi for powerful innovative pre- and post-harvest plant protection. Curr Opin Plant Biol. 38:133-141.
25. Mansfield, J., Genin, S., Magori, S., Citovsky, V., Sriariyanum, M., Ronald, P., Dow, M., Verdier, V., Beer, S V., Machado, M. A., Toth, I., Salmond, G., Foster, G. D. (2012). Top 10 plant pathogenic bacteria in molecular plant pathology. Mol Plant Pathol. 13:614-29.
26. Winston W M, Sutherlin M, Wright A J, Feinberg E H, Hunter C P. (2007). Caenorhabditis elegans SID-2 is required for environmental RNA interference. Proc Natl Acad Sci USA. 104(25):10565-70.
27. Whangbo, J. S. & Hunter, C. P. (2008). Environmental RNA interference. Trends Genet. 24, 297-305.
28. McEwan, D. L., Weisman, A. S. & Huntert, C. P. (2012). Uptake of extracellular double-Stranded RNA by SID-2. Mol. Cell 47, 746-754.
29. Feinberg, E. H. & Hunter, C. P. (2003). Transport of dsRNA into cells by the transmembrane protein SID-1. Science 301, 1545-1547.
30. Ivashuta, S. et al. (2015). Environmental RNAi in herbivorous insects. RNA 21, 840-850
31. Hinas, A., Wright, A. J. & Hunter, C. P. (2012). SID-5 Is an endosome-associated protein required for efficient systemic RNAi in C. elegans. Curr. Biol. 22, 1938-1943.
32. Bolognesi, R., Ramaseshadri, P. Anderson, J., Bachman, P., Clinton, W., Flannagan, R., Ilagan, O., Lawrence, C., Levine, S., Moar, W., Mueller, G., Tan, J., Ullman, J., Wiggins, E., Heck, G., Segers, G. (2012). Characterizing the mechanism of action of double-stranded RNA activity against western corn rootworm (Diabrotica virgifera virgifera LeConte). PloS one, 7(10), e47534.
33. Escobar, M. A., Civerolo, E. L., Summerfelt, K. R., Dandekar, A. M. (2001). RNAi-mediated oncogene silencing confers resistance to crown gall tumorogenesis. Proc. Natl. Acad. Sci. 98: 13437-13442.
34. Zhang, R., Lin, Y. (2009). DEG 5.0, a database of essential genes in both prokaryotes and eukaryotes. Nucleic Acids Res., 37: D455-D458.
35. Cheng, W., Song X. S., Li, H. P., Cao, L. H., Sun, K., Qiu, X. L., Xu, Y. B., Yang, P., Huang, T., Zhang, J. B., Qu, B., Liao, Y. C. (2015). Host-induced gene silencing of an essential chitin synthase gene confers durable resistance to Fusarium head blight and seedling blight in wheat. Plant Biotechnol J., 13:1335-1245.
36. Chen, W., Kastner, C., Nowara, D., Oliveira-Garcia, E., Rutten, T., Zhao, Y., Deising, H. B., Kumlehn, J., Schweizer, P. (2016). Host-induced silencing of Fusarium culmorum genes protects wheat from infection. J Exp Bot., 67: 4979-4991.
37. Panwar, V., Jordan, M., McCallum, B., Bakkeren, G. (2018). Host-induced silencing of essential genes in Puccinia triticina through transgenic expression of RNAi sequences reduces severity of leaf rust infection in wheat. Plant Biotechnol J., 16:1013-1023.
38. Qi, T., Zhu, X., Tan, C., Liu, P., Guo, J., Kang, Z., Guo, J. (2018). Host-induced gene silencing of an important pathogenicity factor PsCPK1 in Puccinia striiformis f. sp. tritici enhances resistance of wheat to stripe rust. Plant Biotechnol J., 16:797-807.
39. Govindarajulu, M., Epstein, L. Wroblewsli, T., Michelmore, R. W. (2015). Host-induced gene silencing inhibits the biotrophic pathogen causing downy mildew of lettuce. Plant Biotechnol J., 13:875-83.
40. Thakare, D., Zhang, J., Wing, R. A., Cotty, P. J., Schmidt, M. A. (2017). Aflatoxin-free transgenic maize using host-induced gene silencing. Sci Adv., 3: e1602382.
41. Dalakouras A., Jarausch W, Buchholz G, Bassler A, Braun M, Manthey T, Krczal G, Wassenegger M. Delivery of Hairpin RNAs and Small RNAs Into Woody and Herbaceous Plants by Trunk Injection and Petiole Absorption. Front Plant Sci. August 24; 9:1253
42. Mitter, N., Worrall, E. A., Robinson, K. E., Li, P., Jain, R. G., Taochy, C., Fletcher, S. J., Carroll, B. J., Lu, G. Q., Xu, Z. P. (2017). Clay nanosheets for topical delivery of RNAi for sustained protection against plant viruses. Nat Plants, 3:16207.
43. Kowal, J., Arras, G., Colombo, M., Jouve, M., Morath, J. P., Primdal-Bengtson, B., Dingli, F., Loew, D., Tkach, M., Thery, C. (2016). Proteomic comparison defines novel markers to characterize heterogenous populations of extracellular vesicle subtypes. Proc Natl Acad Sci USA., 113: E968-77.
44. Rutter, B. D., Innes, R. W. (2017). Extracellular vesicles isolated from the leaf apoplast carry stress-response proteins. Plant Physiol., 173: 728-741.
45. Rutter, B. D., Innes, R. W. (2018). Extracellular vesicles are key mediators of plant-microbe interactions. Current opinion in Plant Biology., 44:16-22.
46. Regente, M., Corti-Monzon, G., Maldonado, A. M., Pinedo, M., Jorrin, J., de la Canal, L. (2009). Vesicular fractions of sunflower apoplastic fluids are associated with potential exosome marker proteins. FEBS Lett., 583:3363-3366. 34.
47. Mathieu, M., Martin Jaular, L., Lavieu, G., Théry, C. (2019). Specificities of secretion and uptake of exosomes and other extracellular vesicles for cell-to-cell communication. Nature cell biology., 9-17.
48. Tkach, M., Thery, C. (2016). Communication by Extracellular Vesicles: Where We Are and Where We Need to Go. Cell: 1226-32.
49. Navarro, L., Jay, F., Nomura, K., He, S. Y. & Voinnet, O. Suppression of the microRNA pathway by bacterial effector proteins. Science 321, 964-7 (2008).
50. Melotto, M., Underwood, W., Koczan, J., Nomura, K., He, S. Y. (2006). Plant stomata function in innate immunity against bacterial invasion. Cell. 126:969-80.
51. Fouts, D. E., Abramovitch, R. B., Alfano, J. R., Baldo, A. M., Buell, C. R., Cartinhour, S., Chatterjee, A. K., D'Ascenzo, M., Gwinn, M. L., Lazarowitz, S. G., Lin, N.C., Martin, G. B., Rehm, A. H., Schneider, D. J., van Dijk, K., Tang, X., Collmer, A. (2002). Genome-wide identification of Pseudomonas syringae pv. tomato DC3000 promoters controlled by the HrpL alternative sigma factor. Proc. Natl. Acad Sci. U.S.A 19:2275-80.
52. Zwiesler-Vollick, J., Plovanich-Jones, A. E., Nomura, K., Bandyopadhyay, S., Joardar, V., Kunkel, B. N., and He S. Y. 2002. Identification of novel hrp-regulated genes through functional genomic analysis of the Pseudomonas syringae pv. tomato DC3000 genome. Mol. Microbiol. 45:1207-1218.
53. Sreedharan, A., Penaloza-Vazquez, A., Kunkel, B. N. & Bender, C. L. (2006). CorR regulates multiple components of virulence in Pseudomonas syringae pv. tomato DC3000. Mol Plant Microbe Interact. 19, 768-79
54. Fan, J., Crooks, C., and Lamb, C. (2008). High-throughput quantitative luminescence assay of the growth in planta of Pseudomonas syringae chromosomally tagged with Photorhabdus luminescens luxCDABE. Plant J. 53, 393-399.
55. E. A. Meighen. (1991). Molecular biology of bacterial bioluminescence Microbiol. Rev., 55 pp. 123-142
56. W. Ma, Andrade, M. O., Farah, C. S. & Wang, N., 2014. The Post-transcriptional Regulator rsmA/csrA Activates T3SS by Stabilizing the 5??? UTR of hrpG, the Master Regulator of hrp/hrc Genes, in Xanthomonas W. Ma, ed. PLoS Pathogens, 10(2), p. e1003945.
57. Oku, T., A. M. Alvarez, and C. I. Kado. (1995). Conservation of the hypersensitivity-pathogenicity regulatory gene hrpX of Xanthomonas campestris and X. oryzae. DNA Sequence 5:245-249.
58. Tang, X., Xiao, Y., Zhou, J M. (2006) Regulation of the type III secretion system in phytopathogenic bacteria. Mol Plant Microbe Interact 19: 1159-1166
59. Wengelnik, K., Van den Ackerveken, G., Bonas, U. (1996) HrpG, a key hrp regulatory protein of Xanthomonas campestris pv. vesicatoria is homologous to two-component response regulators. Molecular plant-microbe interactions 9: 704-712.
60. Wengelnik, K., Rossier, O., Bonas, U. (1999) Mutations in the regulatory gene hrpG of Xanthomonas campestris pv. vesicatoria result in constitutive expression of all hrp genes. Journal of bacteriology 181: 6828-6831.
61. Vicente JG¹, Holub E B. (2013) Xanthomonas campestris pv. campestris (cause of black rot of crucifers) in the genomic era is still a worldwide threat to brassica crops. Mol Plant Pathol. 14(4.2-18.

1. An in vitro method for inhibiting the expression of at least one gene in a target bacterial cell, said method comprising the step of contacting said target bacterial cell with small RNAs, or with compositions containing small RNAs.

2. The method of claim 1, wherein said small RNA is a siRNA or a miRNA inhibiting specifically the expression of a bacterial essential gene or a bacterial virulence gene or an antibacterial resistance gene of a phytopathogenic bacterium.

3. The method according to claim 1 or 2, wherein said target bacterial cell is a cell from a phytopathogenic bacteria which is for example chosen among: Ralstonia solanacearum, Xanthomonas oryzae pathovars, Xanthomonas campestris pathovars, Xanthomonas axonopodis pathovars, Xanthomonas euvesicatoria pathovars, Xanthomonas hostorum pathovars, Pseudomonas syringae pathovars, Pseudomonas viridiflava pathovars, Pseudomonas savastonoi pathovars, Candidatus liberibacter asiaticus, Candidatus liberibacter solanacearum, Acidovorax citrulli, Acidovorax avenae pathovars, Pectobacterium atrosepticum pathovars, Pectobacterium carotovorum pathovars, Pectobacterium sp., Agrobacterium tumefaciens, Dickeya (dadantii and solani), Erwinia amylovora, Clavibacter michiganensis (michiganensis and sepedonicus), Xylella fastidiosa, Pectobacterium (carotovorum and atrosepticum), Streptomyces scabies, Phytoplasma sp., and Spiroplasma sp.

4. The method according to claim 1 or 2, wherein said target bacterial cell is a cell from a plant beneficial bacteria, which is for example chosen among: Bacillus (e.g. Bacillus subtilis), Pseudomonas (e.g. Pseudomonas putida, Pseudomonas stuzeri, Pseudomonas fluorescens, Pseudomonas protegees, Pseudomonas brassicacearum), Rhizobia (e.g. Rhizobium meliloti), Burkholderia (e.g. Burkholderia phytofirmans), Azospirillum (e.g. Azospirillum lipoferum), Gluconacetobacter (e.g. Gluconacetobacter diazotrophicus), Serratia (e.g. Serratia proteamaculans), Stenotrophomonas (e.g. Stenotrophomonas maltophilia), Enterobacter (e.g. Enterobacter cloacae).

5. The method according to any of claims 1 to 4, wherein said small RNAs have a size comprised between 15 and 30 base pairs.

6. In vitro use of a small RNA or of a composition comprising small RNAs, for inhibiting the expression of at least one gene in a target bacterial cell, wherein said target bacterial cell is contacted directly with said small RNA or with said composition.

7. The in vitro use of claim 6, wherein said small RNA is single-stranded or double-stranded.

8. The in vitro use of claim 6, wherein said composition contains plant extracts obtained from producer plant cells that express at least one long dsRNA that exhibit sequence homologies with at least one gene of said bacterial cell.

9. The in vitro use of claim 8, wherein said composition contains total RNAs, or total small RNAs, or apoplastic fluids, or extracellular vesicles, or extracellular free small RNAs, from said plant cells.

10. The in vitro use of claim 8 or claim 9, wherein said producer plant cells are cells from plants chosen in the group consisting of: Tobacco (e.g. Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum), Cottonseed, Cotton, Bean, Banana/plantain, Sorghum, Pea, Sweet potatoes, Soybeans, Cabbage, Cassava, Onion, Melon, Oats, Peanut, Sunflower, Palm oil, Rye, Citrus, Wheat, Peppers, Yams, Olives, Grapes, Sesame, Sugarcane, Sugarbeet, Pea and Coffee, Orange trees, Apple trees, Citrus trees, Olive trees Chrysanthemum, Impatiens, Geranium, Pelargonium, Phlox, Rhododendron anthurium spp, Rose tree, Curcumas, Anthuriums, Begonia, Hibiscus rosa-sinensis, Amaryllis, Calla, Cyclamen, and Dracaena.

11. The in vitro use according to any one of claims 6-10, wherein said small or long RNA inhibits at least one gene encoding a virulence factor or an essential gene or an antibacterial resistance gene if said bacterial cell is pathogenic, or inhibits at least one gene encoding a repressor of growth or a negative regulator of a pathway that is useful for the host if said bacterial cell is beneficial for the host.

12. A method for treating target plants against bacterial infection, said method comprising the step of introducing into a cell of said target plant a long dsRNA molecule targeting specifically at least one virulence bacterial gene or at least one essential bacterial gene or at least one antibacterial resistance gene.

13. A method for treating target plants against bacterial infection, said method comprising the step of delivering small RNAs inhibiting at least one essential or virulence or antibacterial resistance bacterial gene, or a composition containing such small RNAs, on target plant tissues prior to and/or after bacterial infection.

14. The method of claim 13, wherein said composition contains plant extracts obtained from plant cells expressing at least one long dsRNA that is specific to at least one virulence or essential or antibacterial resistance bacterial gene.

15. The method of any one of claim 13-14, wherein said composition contains apoplastic fluids, or extracellular vesicles, or extracellular free small RNAs recovered from said plant extracts.

16. The method of any one of claim 13-15, wherein said composition is a liquid sprayable composition.

17. A recombinant plant RNA virus triggering the in planta production of small RNAs that can inhibit the expression of at least one bacterial gene target.

18. A DNA recombinant vector comprising a DNA polynucleotide sequence encoding long RNAs inhibiting the expression of at least one essential, virulence or antibacterial resistance bacterial gene, wherein said polynucleotide sequence is expressible in plant cells.

19. A transgenic plant comprising the recombinant plant RNA virus of claim 17, or the recombinant vector of claim 18.

20. The transgenic plant of claim 19, stably or transiently expressing a DNA polynucleotide sequence encoding long RNAs inhibiting the expression of at least one essential bacterial gene, virulence bacterial gene or antibacterial resistance gene.

21. The transgenic plant of claim 19, stably or transiently expressing functional small RNAs inhibiting the expression of at least one essential bacterial gene, virulence bacterial gene or antibacterial resistance gene.

22. A phytotherapeutic composition containing a significant amount of small RNAs inhibiting the expression of an essential bacterial gene, or of a virulence bacterial gene or of an antibacterial resistance bacterial gene.

23. The phytotherapeutic composition of claim 22, containing small RNAs that are contained within total RNA extracts, or extracellular vesicles, or apoplastic fluids or extracellular free small RNA extracts from the transgenic plant of claim 19.

24. The phytotherapeutic composition of claim 22 or claim 23, further containing a bactericidal compound.

25. A combination product comprising the phytotherapeutic composition as defined in claim 22 or claim 23, and a bactericidal compound.

26. The use of the phytotherapeutic composition of claim 22-24, or of the combination product of claim 25, for inhibiting or preventing the growth or pathogenicity of bacteria on target plants.

27. The use of claim 26, wherein said phytopathogenic bacteria are chosen among:
Ralstonia solanacearum, Xanthomonas oryzae pathovars, Xanthomonas campestris pathovars, Xanthomonas axonopodis pathovars, Xanthomonas euvesicatoria pathovars, Xanthomonas hostorum pathovars, Pseudomonas syringae pathovars, Pseudomonas viridiflava pathovars, Pseudomonas savastonoi pathovars, Candidatus liberibacter asiaticus, Candidatus liberibacter solanacearum, Acidovorax citrulli, Acidovorax avenae pathovars, Pectobacterium atrosepticum pathovars, Pectobacterium carotovorum pathovars, Pectobacterium sp., Agrobacterium tumefaciens, Dickeya (dadantii and solani), Erwinia amylovora, Clavibacter michiganensis (michiganensis and sepedonicus), Xylella fastidiosa, Pectobacterium (carotovorum and atrosepticum), Streptomyces scabies, Phytoplasma sp., and Spiroplasma sp.

28. The use of claim 26 or 27, wherein said target plants are chosen among Rice, Maize, Barley, Cottonseed, Cotton, Bean, Banana/plantain, Sorghum, Pea, Sweet potatoes, Soybeans, Cabbage, Cassava, Potato, Tomato, Onion, Melon, Oats, Peanut, Sunflower, Palm oil, Rye, Citrus, Wheat, Peppers, Yams, Olives, Grapes, Taro, Tobacco, Sesame, Sugarcane, Sugarbeet, Pea and Coffee, Orange trees, Apple trees, Citrus trees, Olive trees, Chrysanthemum, Impatiens, Geranium, Pelargonium, Phlox, Rhododendron anthurium spp, Rose tree, Curcumas, Anthuriums, Begonia, Hibiscus rosa-sinensis, Amaryllis, Calla, Cyclamen, and Dracaena.

29. A method for manufacturing the phytotherapeutic composition of claim 23, comprising the steps of:
a) generating a recombinant transgenic plant cell producing a siRNA or a miRNA inhibiting specifically a bacterial essential gene or a bacterial virulence gene or an antibacterial resistance gene of a phytopathogenic bacterium,
b) recovering the cell plant extract, or the total RNAs, or apoplastic fluids, or extracellular vesicles, or extracellular free small RNAs from said recombinant plant cells,
c) optionally, adding an excipient or another active principle in said phytotherapeutic composition.

30. The method of claim 29, wherein said recombinant transgenic plant cell is derived from Tobacco (e.g. Nicotiana benthamiana, Nicotiana tobaccum); Taro (Colocasia esculenta); Giger (Zingiber officinale), Arabidopsis (e.g. Arabidopsis thaliana); Tomato (e.g. Lycopersicon esculentum or Solanum lycopersicum); Potato (Solanum tuberosum); Rice (Oryza sativa); Maize (Zea mays); Barley (Hordeum vulgare); Wheat (e.g. Triticum aestivum, Triticum durum).

31. The method of claim 29 or 30, wherein step a) is performed by expressing plant cells with at least one long dsRNA that is specific to said at least one bacterial gene.

32. An in vitro method to identify candidate small RNAs with antibacterial activity, said method comprising the steps of:
a) expressing in plant cells at least one long dsRNA inhibiting at least one bacterial gene,
b) contacting said plant cells with a lysis buffer or with the apoplastic fluid of said plant cells,
c) incubating said plant cell lysates or fluid with target bacterial cells, and
d) assessing the viability, growth, metabolic activity, of said bacterial cells.

33. The method of claim 32, wherein said plant cells are issued from tobacco leaves.

34. An in vitro method to identify candidate genes that affect the proliferation of bacterial cells, said method comprising the steps of:
a) generating small RNAs inhibiting at least one bacterial gene,
b) incubating said small RNAs with bacterial cells, and
c) assessing the viability, growth, metabolic activity, of said bacterial cells.
